(19) United States
(12) Patent Application Publication
(10) Pub. No.: US 2017/0015758 A1
(43) Pub. Date: Jan. 19, 2017

(54) Compositions And Methods For Modulating And Redirecting Immune Responses

(71) Applicants: MEDIMMUNE, LLC, Gaithersburg, MD (US); DUKE UNIVERSITY, Durham, NC (US)

(72) Inventors: Scott A. HAMMOND, Gaithersburg, MD (US); Michael A. MORSE, Durham, NC (US); Takuya OSADA, Durham, NC (US); Herbert Kim LYERLY, Durham, NC (US)

(21) Appl. No.: 15/113,189

(22) PCT Filed: Jan. 21, 2015
(86) PCT No.: PCT/US2015/012149

§ 371 Date: Jul. 21, 2016

Related U.S. Application Data
(60) Provisional application No. 61/929,580, filed on Jan. 21, 2014.

(51) Int. Cl. C07K 16/30; C07K 16/28

(52) U.S. Cl. CPC C07K 16/3007; C07K 16/2809; C07K 2317/31; C07K 2317/626; C07K 2317/622; C07K 2317/54; C07K 2317/35; C07K 2317/55; C07K 2317/732

(57) ABSTRACT

Provided herein are methods of modulating and redirecting an immune response. Compositions and methods for killing targeted cells in a cell population are also provided wherein, a cell population containing target cells expressing a target associated antigen and T cells are contacted with 1, 2, or more immune checkpoint antagonists and a multispecific T cell-redirecting agent that specifically binds the target associated antigen expressed on the target cells and specifically binds a T cell surface antigen.

### REFERENCE TO SEQUENCE LISTING SUBMITTED ELECTRONICALLY

The content of the electronically submitted sequence listing in ASCII text file (Name CEABT-210WO1_SequenceListing.txt; Size: 220,616 bytes; and Date of Creation: Jan. 6, 2014) filed with the application is incorporated herein by reference in its entirety.

### BACKGROUND

Cancer continues to be a major global health burden. Despite progress in the treatment of cancer, there continues to be an unmet medical need for more effective and less toxic therapies, especially for those patients with advanced disease or cancers that are resistant to existing therapeutics.

The role of the immune system, in particular T cell-mediated cytotoxicity, in tumor control is well recognized. There is mounting evidence that T cells control tumor growth and survival in cancer patients, both in early and late stages of the disease. However, tumor-specific T-cell responses are difficult to mount and sustain in cancer patients, and are limited by numerous immune escape mechanisms coopted by tumor cells during immunoediting.

Recent studies suggest that the subversion of immune pathways, termed immune checkpoints, that normally serve to temper T-cell mediated immune responses and control autoimmunity, provide a common mechanism by which tumors are able evade host immune responses. Consequently, much attention has been directed to understanding immune checkpoint pathways with the hope of translating this understanding into the next generation of immunostimulatory drugs. Two T cell inhibitory checkpoint pathways receiving significant attention to date signal through cytotoxic T lymphocyte antigen-4 (CTLA-4, CD152) and programmed death-1 (PD1, CD279), two members of the CD28:B7 superfamily.

CTLA4 is expressed on activated T cells and serves as a co-inhibitor to keep T cell responses in check following CD28-mediated T cell activation. CTLA4 is believed to regulate the amplitude of the early activation of naïve and memory T cells following TCR engagement and to be part of a central inhibitory pathway that affects both antitumor immunity and autoimmunity. CTLA4 is expressed exclusively on T cells and the expression of its ligands B 7.1 (CD80) and B7.2 (CD86), is largely restricted to antigen-presenting cells, T cells, and other immune mediating cells. Antagonistic anti-CTLA4 antibodies that block the CTLA4 signaling pathway have been reported to enhance T cell activation. One such antibody, ipilimumab, was approved by the FDA in 2011 for the treatment of metastatic melanoma.

The PD1/PD-L1 pathway is believed to primarily function to limit autoimmunity by restraining the activity of T cells in the periphery during chronic inflammation, infection and cancer. This pathway is thought to deliver inhibitory signals that predominantly regulate the effector phase of T cells against tumor cells and has been implicated in the phenomena of T-cell “exhaustion” that facilitates an immunosuppressive environment favoring tumor growth and progression. See, e.g., Ribas A., New Engl. J. Med., 367(12):1168 (2012). Consequently, blocking the PD1/PD-L1 pathway provides a promising approach for achieving immunopotentiation in tumor therapy.

PD1 is expressed on activated T cells and regulatory T cells, NK-T cells, B cells, and activated monocytes. PD1 has two potential ligands, PD-L1 and PD-L2. PD-L1 (B7-H1, CD274) is expressed on tumor cells, somatic cells especially in immune privileged sites (e.g., eye, ovary, placenta) and immune cells such as lymphocytes (B and T cells) macrophages and myeloid-derived suppressor cells, to downregulate T cell activity. See, e.g., Sharpe et al., Nat. Immunol. 8:239-245 (2007). Solid tumors such as, melanoma, renal cell carcinoma, and non-small cell lung cancers with elevated PD-L1 expression have shown clinical responses to therapies that disrupt the PD1/PD-L1 immune checkpoint pathway. See, e.g., Ribas A., New Engl. J. Med., 367(12):1168 (2012). PD-L2 expression is restricted to macrophages and dendritic cells (e.g., tolerogenic dendritic cells).

In addition to the numerous escape mechanisms coopted by tumors during immunoediting, the limited number of tumor reactive T cells limit the ability of cancer patients to mount and sustain tumor-specific T cell responses. An alternative approach to engage T cells for cancer therapy involves new classes of antibodies that are bispecific for a tumor associated antigen on cancer cells and for CD3 on T cells. These bispecific antibodies are capable of redirecting the cytotoxic activity of any kind of cytotoxic T cell to a cancer cell independent of T-cell receptor specificity, or peptide antigen presentation. One such class of bispecific antibodies is referred to as bispecific T-cell engager (“BiTE®”) antibodies. BiTE antibodies not only induce the potent lysis of human cancer cell lines in vitro, but have also been shown to mediate the killing of primary human tumor cells by cancer patient T cells ex vivo. See, e.g., Witthauer et al., Breast Cancer Res Treat. 117:471-481 (2009); Wimberger et al., Int J Cancer. 105:241-248 (2003). Furthermore, BiTE antibodies promote in vivo tumor regression in numerous pre-clinical animal models and have shown signs of clinical benefit in patients with cancer. See, e.g., Bargou et al., Science. 321:974-977 (2008); Topp et al., J. Clin. Oncol. 29:2493-2498 (2011); and Nagorsen et al., Exp Cell Res. 317:1255-1260 (2011); Amann et al., Cancer Res. 68:143-151 (2008); Dreier et al., J Immunol. 170:4397-4402 (2003); Hammond et al., Cancer Res. 67:3927-3935 (2007); Herrmann et al., PLoS One. 5:e13474 (2010); Schlereth et al., Cancer Res. 65:2882-2889 (2005).

MEDI-565 (also known as CEA-BiTE; AMG 211; MT111 (SEQ ID NO:3) is a CEA/CD3 BiTE bispecific antibody reported to prevent subcutaneous tumor growth and formation of lung metastases in preclinical models. See, e.g., Lutterbuese et al., 2009. MEDI-565 is in phase I clinical trials (clinicaltrials.gov identifier: NCT01284231) for the treatment of gastrointestinal adenocarcinomas.

Despite the significant progress made over the past decade in developing strategies for combatting cancer and other diseases, patients with advanced, refractory and metastatic disease have limited clinical options. Chemotherapy, irradiation, and high dose chemotherapy have become dose limiting. There remains a substantial unmet need for new less-toxic methods and therapeutics that have better therapeutic efficacy, longer clinical benefit, and improved safety profiles, particularly for those patients with advanced disease or cancers that are resistant to existing therapeutics.

### BRIEF SUMMARY

Compositions and methods for modulating and redirecting immune responses are provided.

In one embodiment, methods are provided for killing targeted cells in a cell population, comprising contacting a cell population containing target cells expressing a target associated antigen and T cells with (a) 1, 2, or more immune checkpoint antagonists (ImCpAnts) that specifically bind 2 or more different targets of an immune checkpoint pathway and (b) a multispecific T cell-redirecting agent (MsTC-Redir) that (i) specifically binds the target associated antigen expressed on the target cells and (ii) specifically binds a T cell surface antigen, wherein the contacting of the cell population with (a) and (b) leads to death of target cells.

An additional embodiment provides a method of killing a tumor cell, comprising contacting a cell population containing tumor cells expressing a tumor associated antigen, and T cells, with (a) 1, 2, or more immune checkpoint antagonists (ImCpAnts) that specifically bind 2 or more different targets of targets of an immune checkpoint pathway and (b) a multispecific T cell-redirecting agent (MsTC-Redir) that (i) specifically binds the tumor associated antigen and (ii) specifically binds a T cell surface antigen, wherein the contacting of the cell population with (a) and (b) leads to death of tumor cells.

Another embodiment provides a method of killing epithelial tumor cells, comprising contacting a cell population containing epithelial tumor cells expressing a tumor associated antigen, and T cells, with (a) 1, 2, or more immune checkpoint antagonists (ImCpAnts) that specifically bind 2 or more different targets of an immune checkpoint pathway, and (b) a multispecific T cell-redirecting agent (MsTC-Redir) that (i) specifically binds the epithelial tumor associated antigen and (ii) specifically binds a T cell surface antigen, wherein the contacting of the cell population with (a) and (b) leads to death of epithelial tumor cells.

A further embodiment provides, a method of killing a CEA (CEACAM5) expressing tumor cell, comprising contacting a cell population containing tumor cells expressing CEA, and T cells with (a) 1, 2, or more immune checkpoint antagonists (ImCpAnts) that specifically bind 2 or more different targets of an immune checkpoint pathway, and (b) a multispecific T cell-redirecting agent (MsTC-Redir) that (i) specifically binds CEA and (ii) specifically binds a T cell surface antigen, wherein the contacting of the cell population with (a) and (b) leads to death of CEA expressing tumor cells.

In some embodiments of the methods described herein, the cell population is contacted with 1, 2 or more ImCpAnts before the cell population is contacted with the MsTC-Redir. In additional embodiments, the cell population is contacted with 1, 2 or more ImCpAnts that specifically bind 2 or more different targets of an immune checkpoint pathway before the cell population is contacted with the MsTC-Redir. In some embodiments, the cell population is contacted with 1, 2 or more ImCpAnts at about ½, 1, 2, 3, 4, 5, 6, 7, 8, 9, 10, 11, 12, 18, 24, 36, 48, 60 or 96 hours before the cell population is contacted with the MsTC-Redir. In additional embodiments, the cell population is contacted with 1, 2 or more ImCpAnts at about ½ hour to about 3 weeks, about ½ hour to about 2 weeks or about ½ hour to about 1 week before the cell population is contacted with the MsTC-Redir. In further embodiments, the cell population is contacted with 1, 2 or more ImCpAnts that specifically bind 2 or more different targets of an immune checkpoint pathway at about ½ hour to about 3 weeks, about ½ hour to about 2 weeks or about ½ hour to about 1 week before the cell population is contacted with the MsTC-Redir. In additional embodiments, the cell population is contacted with 1, 2 or more ImCpAnts at about the same time as the cell population is contacted with the MsTC-Redir. In further embodiments, the cell population is contacted with 1, 2 or more ImCpAnts that specifically bind 2 or more different targets of an immune checkpoint pathway, at about the same time as the cell population is contacted with the MsTC-Redir. In additional embodiments, the cell population is contacted with 1, 2 or more ImCpAnts within 6 hours of the cell population being contacted with the MsTC-Redir. In further embodiments, the cell population is contacted with 1, 2 or more ImCpAnts that specifically bind 2 or more different targets of an immune checkpoint pathway within 6 hours of the cell population being contacted with the MsTC-Redir.

In some embodiments, an immune checkpoint pathway targeted using the methods disclosed herein is the PD1/PDL1 or CTLA4 immune checkpoint pathway. In additional embodiments, the methods provide the use of ImCpAnts, such as monoclonal antibodies, or antigen binding fragments thereof, that specifically bind one, two, three or more targets selected from PD1, PD-L1, PD-L2, CTLA4, B7.1, B7.2 and B7H2. In some embodiments, the ImCpAnts specifically bind two or more targets in an immune checkpoint pathway. In some embodiments, the ImCpAnts specifically bind two or more targets in different checkpoint pathways.

In some embodiments, the ImCpAnts include an antagonist anti-PD1 antibody and an antagonist anti-PD-L1 antibody. In additional embodiments, the ImCpAnts include 1, 2 or more of: (a) an anti-PD1 antibody or antigen binding fragment thereof, an anti-PD-L1 antibody or antigen binding fragment thereof, and/or an anti-PD-L2 antibody or antigen binding fragment thereof, and/or (b) an anti-CTLA4 antibody or antigen binding fragment thereof, an anti-B7.1 antibody or antigen binding fragment thereof, an anti-B7.2 antibody or antigen binding fragment thereof, and/or an anti-B7H2 antibody or antigen binding fragment thereof.

Additional immune checkpoint pathways that can be targeted using the methods disclosed herein include, but are not limited to, an immune checkpoint pathway selected from: the BTLA (B- and T lymphocyte attenuator; also known as CD272), PDH1 (also known as V-domain Ig suppressor of T cell activation; VISTA), B7H3-TLT2 (also known as CD276), B7H4 (VCTN1), TIM3 (T cell immunoglobulin mucin 3; also known as HAVcr2), A2aR (adenosine A2a receptor), and the LAG3 (lymphocyte activation gene 3; also known as CD223) immune checkpoint pathway. In additional embodiments, the methods provide the use of ImCpAnts, such as monoclonal antibodies, or antigen binding fragments thereof, that specifically bind one, two, three or more targets selected from BTLA, PDH1, B7H3, B7H4, TIM3, A2aR, and LAG3. In some embodiments, the ImCpAnts specifically bind two or more targets in an immune checkpoint pathway. In some embodiments, the ImCpAnts specifically bind two or more targets in different checkpoint pathways.

In some embodiments, the disclosed methods include the step of contacting the cell populations with agonists of one, two, three or more immune activating pathway (i.e., immune activating agonists (ImActAgs)). In some embodiments, the ImActAgs are monoclonal antibodies or antigen binding fragments thereof that specifically bind one, two, three or more receptors or ligands in an immune activating pathway selected from the 4-1BB/CD137-CD137L, OX40-OX40L, GITRL-GITR, CD27-CD70, CD28-ICOS or the HVEM-LIGHT pathway. In additional embodiments, one, two, three or more ImActAgs is an antibody that is an Fc fusion protein comprising an IgG Fc region fused to one or more polypeptides such as, a portion of immune activating pathway protein, an scFv, or a synthetic peptide that binds an immune activating pathway protein. In additional embodiments, the disclosed methods include the step of contacting the cell populations with one, two, three or more ImActAgs that specifically bind one, two, three or more targets selected from 4-1BB/CD137, CD137L, OX40, OX40L, GITRL, GITR, CD27, CD70, CD28, ICOS, HVEM, and LIGHT. In some embodiments, the ImActAgs specifically bind two or more targets in an immune activating pathway. In some embodiments, the ImActAgs specifically bind two or more targets in different immune activating pathways.

In additional embodiments of the methods described herein, the MsTC-Redir binds the CD3/TCR complex expressed on the surface of a T cell. In some embodiments, the MsTC-Redir is a bispecific antibody. In further embodiments, bispecific antibody is a member selected from the group consisting of a bispecific diabody, a single-chain bispecific diabody, a single chain bispecific tandem variable domain, a bispecific single domain antibody, a bispecific F(ab′)₂, a dock-and-lock bivalent or trivalent Fab, a bispecific (mab)₁, and a bispecific (mab)₂. In additional embodiments, the bispecific antibody is a bi-specific T-cell engager (BiTE). In further embodiments, the BiTE competes for binding with CD3 and/or CEA (CEACAM5) with an antibody or antigen binding fragment thereof comprising the amino acid sequence of SEQ ID NO:3. In additional embodiments the BiTE binds to the same epitope of CD3 and/or CEA as an antibody or antigen binding fragment thereof comprising the amino acid sequence of SEQ ID NO:3. In further embodiments, the BiTE is an antibody or antigen binding fragment thereof, comprising the amino acid sequence of SEQ ID NO:3.

In further embodiments, the BiTE competes for binding with CD3 and/or CEA (CEACAM5) with an antibody or antigen binding fragment thereof comprising the amino acid sequence of SEQ ID NO:16. In additional embodiments the BiTE binds to the same epitope of CD3 and/or CEA as an antibody or antigen binding fragment thereof comprising the amino acid sequence of SEQ ID NO:16. In further embodiments, the BiTE is an antibody or antigen binding fragment thereof, comprising the amino acid sequence of SEQ ID NO:16.

In some embodiments of the methods described herein, the methods target tumor cells, immune cells, or an infectious agent. In additional embodiments, the tumor cells express CEA (CEACAM5). In additional embodiments, the tumor cells are from an epithelial tumor of the gastrointestinal tract. In other embodiments, the tumor cells are from a melanoma, renal cell carcinoma, non-small cell lung cancer, colon cancer, pancreatic cancer, esophageal cancer, gastric cancer or a colorectal cancer.

The method described herein can be performed in vitro, ex vivo, or in vivo.

In another embodiment, the compositions and methods provide a method of modulating (e.g., increasing) and redirecting an immune response to a diseased cell or tissue and/or an immune cell in a subject, comprising, administering to the subject (a) 1, 2, or more immune checkpoint antagonists (ImCpAnts) that specifically bind 2 or more different targets of an immune checkpoint pathway and (b) a multispecific T cell-redirecting agent (MsTC-Redir) that (i) specifically binds an antigen on the surface of the diseased cell or tissue and/or an immune cell and (ii) specifically binds a T cell surface antigen. In some embodiments, the immune response is redirected to a diseased cell, tumor cell, immune cell, or an infectious agent.

An additional embodiment provides a method of treating a tumor in a subject, comprising administering to the subject (a) 1, 2, or more immune checkpoint antagonists (ImCpAnts) that specifically bind 2 or more different targets of an immune checkpoint pathway and (b) a multispecific T cell-redirecting agent (MsTC-Redir) that (i) specifically binds an antigen on the tumor cell surface and (ii) specifically binds a T cell surface antigen. In further embodiments, the tumor cell or tumor expresses CEA (CEACAM5). In additional embodiments, the tumor cell or tumor is an epithelial tumor of the gastrointestinal tract. In other embodiments, the tumor cell or tumor is a melanoma, renal cell carcinoma, non-small cell lung cancer, colon cancer, pancreatic cancer, esophageal cancer, gastric cancer or a colorectal cancer.

An additional embodiment provides a method of treating an epithelial tumor in a subject, comprising administering to the subject (a) 1, 2, or more immune checkpoint antagonists (ImCpAnts) that specifically bind 2 or more different targets of an immune checkpoint pathway, and (b) a multispecific T cell-redirecting agent that (i) specifically binds an epithelial tumor associated antigen and (ii) specifically binds a T cell surface antigen.

A further method provides a method of treating a tumor containing cells expressing cell surface CEA (CEACAM5) in a subject, comprising administering to the subject (a) 1, 2, or more immune checkpoint antagonists (ImCpAnts) that specifically bind 2 or more different targets of an immune checkpoint pathway, and (b) a multispecific T cell-redirecting agent (MsTC-Redir) that (i) specifically binds CEA and (ii) specifically binds a T cell surface antigen.

In some embodiments, the disclosed methods provide the use/administration of a multispecific T cell-redirecting agent (MsTC-Redir) and one, two or more immune checkpoint antagonists (ImCpAnts) that specifically bind and inhibit the signaling of two or more members of an immune checkpoint pathway. In some embodiments the MsTC-Redir is a bispecific tandem scFv (discFv). In further embodiments, the bispecific tandem di-scFv is a bi-specific T-cell engager (BiTE). In other embodiments, the MsTC-Redir is a diabody.

An additional embodiment provides a method of enhancing antitumor immunity in a subject comprising co-administering to a subject a bi-specific T-cell engager (BiTE) and two or more ImCpAnts. In some embodiments, the co-administered BiTE competes for binding with CD3 and/or CEA (CEACAM5) with an antibody or antigen binding fragment thereof comprising the amino acid sequence of SEQ ID NO:3. In additional embodiments the BiTE binds to the same epitope of CD3 and/or CEA as an antibody or antigen binding fragment thereof comprising the amino acid sequence of SEQ ID NO:3. In further embodiments, the BiTE is an antibody or antigen binding fragment thereof, comprising the amino acid sequence of SEQ ID NO:3.

An additional embodiment provides a method of enhancing antitumor immunity in a subject comprising co-administering to a subject a bi-specific T-cell engager (BiTE) and two or more ImCpAnts. In some embodiments, the co-administered BiTE competes for binding with CD3 and/or CEA (CEACAM5) with an antibody or antigen binding fragment thereof comprising the amino acid sequence of SEQ ID NO:16. In additional embodiments the BiTE binds to the same epitope of CD3 and/or CEA as an antibody or antigen binding fragment thereof comprising the amino acid sequence of SEQ ID NO:16. In further embodiments, the BiTE is an antibody or antigen binding fragment thereof, comprising the amino acid sequence of SEQ ID NO: 16. An additional method provides a method of reducing resistance of a tumor cell to T cell mediated killing in a subject, comprising co-administering to a subject a bi-specific T-cell engager (BiTE) and two or more ImCpAnts. In some embodiments, the co-administered BiTE competes for binding with CD3 and/or CEA (CEACAM5) with an antibody or antigen binding fragment thereof comprising the amino acid sequence of SEQ ID NO:3. In additional embodiments the BiTE binds to the same epitope of CD3 and/or CEA as an antibody or antigen binding fragment thereof comprising the amino acid sequence of SEQ ID NO:3. In further embodiments, the BiTE is an antibody or antigen binding fragment thereof, comprising the amino acid sequence of SEQ ID NO:3. In additional embodiments, the BiTE is an antibody or antigen binding fragment thereof, comprising the amino acid sequence of SEQ ID NO:16.

In additional embodiments of the methods described herein, the MsTC-Redir binds the CD3/TCR complex expressed on the surface of a T cell. In some embodiments, the MsTC-Redir is a bispecific antibody. In further embodiments, bispecific antibody is a member selected from the group consisting of a bispecific diabody, a single-chain bispecific diabody, a single chain bispecific tandem variable domain, a bispecific single domain antibody, a bispecific F(ab′)₂, a dock-and-lock bivalent or trivalent Fab, a bispecific (mab)₁, and a bispecific (mab)₂. In additional embodiments, the bispecific antibody is a bi-specific T-cell engager (BiTE). In further embodiments, the BiTE competes for binding with CD3 and/or CEA (CEACAM5) with an antibody or antigen binding fragment thereof comprising the amino acid sequence of SEQ ID NO:3. In additional embodiments the BiTE binds to the same epitope of CD3 and/or CEA as an antibody or antigen binding fragment thereof comprising the amino acid sequence of SEQ ID NO:3. In further embodiments, the BiTE is an antibody or antigen binding fragment thereof, comprising the amino acid sequence of SEQ ID NO:3.

In further embodiments the BiTE binds to the same epitope of CD3 and/or CEA as an antibody or antigen binding fragment thereof comprising the amino acid sequence of SEQ ID NO:16. In further embodiments, the BiTE is an antibody or antigen binding fragment thereof, comprising the amino acid sequence of SEQ ID NO:16.

In some embodiments, an immune checkpoint pathway targeted using the methods disclosed herein is the PD1/PDL1 or CTLA4 immune checkpoint pathway. In additional embodiments, the methods provide the use of ImCpAnts, such as monoclonal antibodies, or antigen binding fragments thereof, that specifically bind one, two, three or more targets selected from PD1, PD-L1, PD-L2, CTLA4, B7.1, B7.2 and B7H2. In some embodiments, the ImCpAnts specifically bind two or more targets in an immune checkpoint pathway. In some embodiments, the ImCpAnts specifically bind two or more targets in different checkpoint pathways.

In additional embodiments the ImCpAnts used/administered according to the disclosed methods include antagonists to two different targets on the PD1-PD-L1 immune checkpoint pathway. In particular embodiments, the ImCpAnts are a PD-L1 antagonist and a PD1 antagonist. In additional embodiments, the ImCpAnts include 1, 2 or more of: (a) an anti-PD1 antibody or antigen binding fragment thereof, an anti-PD-L1 antibody or antigen binding fragment thereof, and/or an anti-PD-L2 antibody or antigen binding fragment thereof, and/or (b) an anti-CTLA4 antibody or antigen binding fragment thereof, an anti-B7.1 antibody or antigen binding fragment thereof, an anti-B7.2 antibody or antigen binding fragment thereof, and/or an anti-B7H2 antibody or antigen binding fragment thereof.

Additional immune checkpoint pathways that can be targeted using the methods disclosed herein include, but are not limited to, an immune checkpoint pathway selected from: the BTLA (B- and T lymphocyte attenuator; also known as CD272), PDH1 (also known as V-domain Ig suppressor of T cell activation; VISTA), B7H3-TLT2 (also known as CD276), B7H4 (VCTN1), TIM3 (T cell immunoglobulin mucin 3; also known as HAVcr2), A2aR (adenosine A2a receptor), and the LAG3 (lymphocyte activation gene 3; also known as CD223) immune checkpoint pathway. In additional embodiments, the methods provide the use of ImCpAnts, such as monoclonal antibodies, or antigen binding fragments thereof, that specifically bind one, two, three or more targets selected from BTLA, PDH1, B7H3, B7H4, TIM3, A2aR, and LAG3. In some embodiments, the ImCpAnts specifically bind two or more targets in an immune checkpoint pathway. In some embodiments, the ImCpAnts specifically bind two or more targets in different checkpoint pathways.

In some embodiments, the disclosed methods include the step of administering agonists of one, two, three or more immune activating pathways (i.e., immune activating agonists (ImActAgs)). In some embodiments, the ImActAgs are monoclonal antibodies or antigen binding fragments thereof that specifically bind one, two, three or more receptors or ligands in an immune activating pathway selected from the 4-1BB/CD137-CD137L, OX40-OX40L, GITRL-GITR, CD27-CD70, CD28-ICOS or the HVEM-LIGHT pathway. In additional embodiments, one, two, three or more ImActAgs is an antibody that is an Fc fusion protein comprising an IgG Fc region fused to one or more polypeptides such as, a portion of immune activating pathway protein, an scFv, or a synthetic peptide that binds an immune activating pathway protein. In additional embodiments, the disclosed methods include the step of administering one, two, three or more ImActAgs that specifically bind one, two, three or more targets selected from 4-1BB/CD137, CD137L, OX40, OX40L, GITRL, GITR, CD27, CD70, CD28, ICOS, HVEM, and LIGHT. In some embodiments, the ImActAgs specifically bind two or more targets in an immune activating pathway. In some embodiments, the ImActAgs specifically bind two or more targets in different immune activating pathways.

In some embodiments of the methods described herein, the subject is administered 1, 2 or more ImCpAnts before the subject is administered the MsTC-Redir. In further embodiments, the subject is administered 1, 2 or more ImCpAnts that specifically bind 2 or more different targets of an immune checkpoint pathway before the subject is administered the MsTC-Redir. In additional embodiments, the subject is administered 1, 2 or more ImCpAnts at about ½, 1, 2, 3, 4, 5, 6, 7, 8, 9, 10, 11, 12, 18, 24, 36, 48, 60 or 96 hours before the cell population is contacted with the MsTC-Redir.

In additional embodiments, the subject is administered 1, 2 or more ImCpAnts at about ½ hour to about 3 weeks, about ½ hour to about 2 weeks or about ½ hour to about 1 week before the subject is administered the MsTC-Redir. In further embodiments, the subject is administered 1, 2 or more ImCpAnts that specifically bind 2 or more different targets of an immune checkpoint pathway at about ½ hour to about 3 weeks, about ½ hour to about 2 weeks or about ½ hour to about 1 week before the subject is administered the MsTC-Redir. In some embodiments, the subject is administered 1, 2 or more ImCpAnts at about the same time as the subject is administered the MsTC-Redir. In further embodiments, the subject is administered 1, 2 or more ImCpAnts that specifically bind 2 or more different targets of an immune checkpoint pathway at about the same time as the subject is administered the MsTC-Redir. In other embodiments, the subject is administered 1, 2 or more ImCpAnts within 6 hours of the subject being administered the MsTC-Redir. In further embodiments, the subject is administered 1, 2 or more ImCpAnts that specifically bind 2 or more different targets of an immune checkpoint pathway within 6 hours of the subject being administered the MsTC-Redir.

### BRIEF DESCRIPTION OF THE DRAWINGS/FIGURES

FIG. 1 shows that surviving Tumor cells in 1^{st} round of MEDI-565-mediated T cell killing do not acquire resistance for T cell killing and have enhanced CEA expression. SW1463 tumor cells (A) or AsPC-1 cells (B) were incubated at 37° C. with T cells (E:T ratio=5) and MEDI-565 (100 ng/mL) as described in the Materials and Methods. After 5 days incubation, floating dead cells were discarded, and alive adherent tumor cells were harvested and transferred to 12 well plate for a 2^{nd} round of MEDI-565/T cell attack. For a negative control, tumor cells alone or tumor cells with Cont-BiTE and T cells were incubated for the same period of time. Cytotoxicity was analyzed by staining cells with FITC-conjugated anti-CD3, 7-AAD and APC-conjugated annexin V. Tumor cells (CD3-negative) were analyzed for their annexin V positivity (% shown in each dot plot). (C) Change of CEA expression level after MEDI-565-mediated T cell attack. CEA expressing colorectal cancer cell lines (SW1463, Colo205, and HT29) were incubated with MEDI-565/T cells for 5 days. As control, tumor cells were incubated alone or with Cont-BiTE/T cells for the same period of time. Alive cells from the 1^{st} round of MEDI-565-mediated T cell killing were put into a 2^{nd} round incubation with fresh T cells and MEDI-565 and incubated at 37° C. for another 5 days. The change in CEA expression level was analyzed by flow cytometry. Indirect staining method was used for detection of CEA expression with PE conjugated goat anti-mouse IgG as a secondary antibody. FITC-conjugated anti-CD3 was added to eliminate T cells from the analysis. Filled histograms: anti-CEACAM5 mAb. Open histograms: Isotype control. Percentage of positive cells and median fluorescence intensities are shown.

FIG. 2 shows the temporal Changes in PD1 expression levels on T cells and in PD-L1 expression on cancer cells following CEA-BiTE-mediated T cell killing. T cells were incubated with SW1463 cells in the presence of CEA-BiTE or Cont-BiTE (100 ng/mL) for up to 7 days. After a 1, 3, 5 or 7 day incubation period, cells were harvested and stained with APC-anti-CD45, and PE-conjugated anti-PD1, or anti-PD-L1 mAb. (A) CD45-positive cells (T cell) and (B) CD45-negative (Tumor) cells were analyzed for their PD1 and PD-L1 expression, respectively. Open histograms show staining with isotype control IgG.

FIG. 3 shows that metastatic cancer cells from colorectal cancer patients do not acquire resistance for T cell killing and have enhanced CEA expression. (A) The cytotoxicity assay was performed with CEA-positive colorectal cancer cells derived from metastatic colorectal cancer lesions in patients CRC057 and CRC096. Cancer cells that survived a single round of MEDI-565-mediated T cell killing were harvested, and used for a second round of incubation with MEDI-565 and T cells. After 5 days incubation, tumor cells were analyzed for annexin-V and 7-AAD labeling as conducted and shown in FIG. 1. Percentages of annexin V-positive cells versus 7-AAD are shown in each dot plot. (B) Levels of CEA expression after MEDI-565-mediated T cell killing were analyzed by flow cytometry. Representative staining with CRC057 cells are shown. Filled histograms: anti-CEACAM5 mAb. Open histograms: Isotype control. Median fluorescence intensities are shown.

FIG. 4 shows the blocking effect of PD1 and PD-L1 on CEA-BiTE/T cell mediated killing with exhausted T cells. Effect of anti-PD1, anti-PD-L1, or combination of these antibodies on CEA-BiTE-mediated killing with T cells was analyzed. T cells were incubated with SW1463 cells in the presence of CEA-BiTE or Cont-BiTE (100 ng/mL) for 7 days. T cells were harvested as described in Materials and Methods and used as effector cells in the 2^{nd} round incubation of CEA-BiTE-mediated T cell killing against SW1463 cells. After 5 days incubation, cells were stained with anti-CD3-FITC/7-AAD/annexin-V-APC. CD3-negative FSC large tumor cells were analyzed for annexin V and 7-AAD labeling. Percentages of annexin V-positive cells in tumor cells are shown in each dot plot.

FIG. 5 shows the decreased killing activity of T cells after incubation with MEDI-565 and cancer cells. Tumor cells were incubated with T cells and MEDI-565 for 5 days, then floating cells were harvested, washed with PBS twice, and single cell suspensions were obtained by repeated gentle pipetting. Viable cells were isolated by density gradient centrifugation with Ficoll-Paque. Harvested viable cells were resuspended in medium and transferred into tissue culture treated flasks to allow the tumor cells to adhere. After a 2 hour incubation period, non-adherent cells were harvested and used as T cells from the MEDI-565/tumor cell culture. Fresh T cells were isolated from frozen PBMCs of the same normal donor using two methods; flask adherence to remove monocytes and a negative T cell isolation kit. T cells incubated alone in the flask for 5 days were also used. After a 5 day incubation period in the presence of MEDI-565 or Cont-BiTE (100 ng/mL), cytotoxic activity of the T cells were compared by staining cells with FITC-conjugated anti-CD3, 7-AAD and annexin V. Tumor cells (CD3-negative) were analyzed for their annexin V positivity (% shown in each dot plot). (A) SW1463 cells as target cells. (B) AsPC-1 cells as target cells.

FIG. 6 shows the increased Regulatory T cell population after CEA-BiTE-mediated tumor cell killing. T cells were incubated with tumor cells (SW1463 or AsPC-1) in the presence of CEA-BiTE or Cont-BiTE (100 ng/mL) for 5 days, harvested and stained with FITC-anti-CD25, PE-anti-Foxp3, PerCP-anti-CD4, and APC-anti-CD3 antibodies after permeabilization. CD25/Foxp3 expression in CD4-positive T cells is shown. Percentages of each quadrant are shown.

FIG. 7 shows the increased PD1 expression on T cells and PD-L1 expression on cancer cells are specific to coincubation in the presence of CEA-BiTE. T cells were incubated with tumor cells (SW1463 or AsPC-1) in the presence of CEA-BiTE or Cont-BiTE (100 ng/mL) for 5 days. Cells were harvested and stained with APC-anti-CD45, and PE-conjugated anti-CD28, CTLA-4, PD1, PD-L1, or CD69. CD45+(T cell) and CD45⁻ (Tumor) cell populations were analyzed for expression levels of these cell surface molecules. Open histograms show staining with PE-conjugated isotype control IgG. Median fluorescence intensity of staining is shown in each histogram.

FIG. 8 shows the effect of PD1 on CEA-BiTE/T cell mediated killing and PD-L1 expression on tumor cells. (A) SW1463 cells and T cells from a healthy donor were cultured (E:T ratio=5) alone, with Cont-BiTE (100 ng/mL) or with CEA-BiTE (100 ng/mL). An anti-PD1 blocking antibody or isotype control IgG was added (final 5 μg/mL) to the cultures. The assay was incubated for 5 days, and cells were harvested and stained with anti-CD3-FITC/7-AAD/Annexin V-APC. (B) Cells were stained with anti-CD3-FITC and PE-labeled anti-PD-L1 or PE-labeled control IgG. CD3-negative tumor cells were gated for the analysis. Filled histogram: PD-L1, Open histogram: control IgG.

FIG. 9 shows the effect of PD1 and PD-L1 on CEA-BiTE/T cell mediated killing with exhausted T cells. (A) SW1463 cells and T cells from a healthy donor were cultured (E:T ratio=5) alone, with Cont-BiTE (100 ng/mL) or with CEA-BiTE (100 ng/mL). Cultures were incubated for 5 days in the presence of anti-PD1 mAb, anti-PD1/anti-PD-L1 mAbs or isotype control IgG (final concentration of 5 μg/mL). Tumor cells were harvested and stained with anti-CD3-FITC/7-AAD/annexin V-APC. (B) T cells from the same normal donor were incubated with SW1463 tumor cells at a 5:1 effector-target ratio in the presence of CEA-BiTE or Cont-BiTE (100 ng/mL) for 5 days. Floating cells were harvested, single cell suspensions were obtained by gentle pipetting, and viable cells were isolated with gradient density centrifugation. Isolated T cells were used for a second round of CEA-BiTE-mediated T cell killing in the presence of anti-PD1 mAb, anti-PD1/anti-PD-L1 mAbs or isotype control IgG (final concentration of 5 μg/mL). After a 5 day-incubation period, tumor cells were harvested and stained with anti-CD3-FITC/7-AAD/annexin V-APC. Percentages of annexin V-positive cells within the CD3-negative tumor cell population are shown in each dot plot.

### DETAILED DESCRIPTION

The present disclosure provides compositions and methods for modulating (e.g., increasing) and redirecting immune responses using 1, 2, or more immune checkpoint antagonists (ImCpAnts) that specifically bind 2 or more different members (targets) of an immune checkpoint pathway. The compositions and methods further include the use of a multispecific T cell-redirecting agent that specifically binds a T cell surface antigen and also specifically binds an antigen on a cell or tissue to which the immune response is to be targeted. Further provided are methods of killing tumor cells and methods of treating tumors in a subject using the compositions and delivery regimens described herein.

In order that the present invention can be more readily understood, certain terms are first defined. Additional definitions are set forth throughout the detailed description.

### I. DEFINITIONS

It is to be noted that the term “a” or “an” entity refers to one or more of that entity; for example, “an anti-PD-L1 antibody” is understood to represent one or more anti-PD-L1 antibodies. As such, the terms “a” (or “an”), “one or more,” and “at least one” can be used interchangeably herein.

Furthermore, “and/or” where used herein is to be taken as specific disclosure of each of the two specified features or components with or without the other. Thus, the term and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of ordinary skill with a general dictionary of many of the terms used in this disclosure.

It is understood that wherever aspects are described herein with the language “comprising,” otherwise analogous aspects described in terms of “consisting of” and/or “consisting essentially of” are also provided.

Amino acids are referred to herein by either their commonly known three letter symbols or by the one-letter symbols recommended by the IUPAC-IUB Biochemical Nomenclature Commission. Nucleotides, likewise, are referred to by their commonly accepted single-letter codes.

The terms “inhibit,” “block,” “blockade” and “suppress” are used interchangeably herein and refer to any statistically significant decrease in biological activity, including full blocking of the activity. For example, “inhibition” can refer to a decrease of at least 10%, or at least 20%, or at least 30%, or at least 40%, or at least 50%, or at least 60%, or at least 70%, or at least 80%, or at least 90%, or about 100% in biological activity. Accordingly, when the terms “inhibition” or “suppression” are applied to describe for example, an effect on PD1 expression on T cells and/or T cell-mediated cytolytic activity, the term refers to for example, the ability of an antagonist such as, an anti-PD1 antibody, to statistically significantly decrease the activity of the antigen to which the antagonist binds. For example the term inhibit or block may be used to refer to the ability of an anti-PD1 antibody to decreased the expression of PD1 on T cells and/or the ability of the anti-PD1 antibody to increase T cell-mediated cytolytic activity in vitro or in vivo, relative to PD1 expression on T cells and/or T cell-medicated cytolytic activity in an untreated cell population (control).

The term “inhibit activation” or “suppress activation” of an effector cell such as a T cell as used herein, refers to the ability of a composition disclosed herein such as, an anti-PD1 antibody, an anti-PD-L1 antibody, and anti-CTLA4 antibody, to statistically significantly decrease the activation of an effector cell expressing the surface antigen (e.g., a T cell) relative to the activation of the effector cell in the absence of the antagonist antibody. In one embodiment, the activation of a T cell or other effector cell expressing the surface antigen is decreased by at least 10%, or at least 20%, or at least 30%, or at least 40%, or at least 50%, or at least 60%, or at least 70%, or at least 80%, or at least 90%, or about 100% when cells are contacted with the antagonist antibody, relative to the activation measured in the absence of the antagonist antibody.

Effector cell activation can be assayed using techniques disclosed herein or other known in the art that measure for example, surface marker expression, intracellular signaling, rates of cell division, cytolytic activity and/or cytokine production.

The term “inhibit proliferation” of a cell expressing a surface antigen (e.g., CEA) as used herein, refers to the ability of a composition disclosed herein such as a bispecific antibody (e.g., CEA-BiTE), to statistically significantly decrease proliferation of a cell expressing the surface antigen (e.g., CEA) relative to the proliferation in the absence of the bispecific antibody (e.g., CEA-BiTE). In one embodiment, the proliferation of a cell expressing the surface antigen is decreased by at least 10%, or at least 20%, or at least 30%, or at least 40%, or at least 50%, or at least 60%, or at least 70%, or at least 80%, or at least 90%, or about 100% when cells are contacted with bispecific antibody (e.g., CEA-BiTE) in the presence of T cells, relative to the proliferation measured in the presence of T cells, but in the absence of the bispecific antibody (e.g., CEA-BiTE) (control conditions). Cellular proliferation can be assayed using art recognized techniques that measure rates of cell division, fractions of cells within a cell population undergoing cell division, and/or rates of cell loss from a cell population due to terminal differentiation or cell death (e.g., thymidine incorporation).

The term “antibody” means an immunoglobulin molecule that recognizes and specifically binds to a target, such as a protein, polypeptide, peptide, carbohydrate, polynucleotide, lipid, or combinations of the foregoing through at least one antigen recognition site within the variable region of the immunoglobulin molecule. As used herein, the term “antibody” encompasses intact polyclonal antibodies, intact monoclonal antibodies, antibody fragments (such as Fab, Fab′, F(ab′)2, and Fv fragments), single chain Fv (scFv) mutants, multispecific antibodies such as bispecific antibodies generated from at least two intact antibodies, chimeric antibodies, humanized antibodies, human antibodies, fusion proteins comprising an antigen determination portion of an antibody, and any other modified immunoglobulin molecule comprising an antigen recognition site so long as the antibodies exhibit the desired biological activity. An antibody can be of any the five major classes of immunoglobulins: IgA, IgD, IgE, IgG, and IgM, or subclasses (isotypes) thereof (e.g., IgG1, IgG2, IgG3, IgG4, IgA1 and IgA2), based on the identity of their heavy-chain constant domains referred to as alpha, delta, epsilon, gamma, and mu, respectively. The different classes of immunoglobulins have different and well known subunit structures and three-dimensional configurations. Antibodies can be naked or conjugated to other molecules such as toxins, radioisotopes, etc. to form Antibody Drug Conjugates (ADC).

The terms “antibody” or “immunoglobulin,” are used interchangeably herein, and include whole antibodies and any antigen binding fragment or single chains thereof. A typical antibody comprises at least two heavy (H) chains and two light (L) chains interconnected by disulfide bonds. Each heavy chain is comprised of a heavy chain variable region (abbreviated herein as VH) and a heavy chain constant region. The heavy chain constant region is comprised of three domains, CH1, CH2, and CH3. Each light chain is comprised of a light chain variable region (abbreviated herein as VL) and a light chain constant region. The light chain constant region is comprised of one domain, CL. The VH and VL regions can be further subdivided into regions of hypervariability, termed Complementarity Determining Regions (CDR), interspersed with regions that are more conserved, termed framework regions (FW). Each VH and VL is composed of three CDRs and four FWs, arranged from amino-terminus to carboxy-terminus in the following order: FW1, CDR1, FW2, CDR2, FW3, CDR3, FW4. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (C1q) of the classical complement system. Exemplary antibodies of the present disclosure include typical antibodies, scFvs, and combinations thereof where, for example, an scFv is covalently linked (for example, via peptidic bonds or via a chemical linker) to the N-terminus of either the heavy chain and/or the light chain of a typical antibody, or intercalated in the heavy chain and/or the light chain of a typical antibody. Additional exemplary “antibodies” herein include fusion proteins comprising an antibody portion, and any other modified immunoglobulin molecule comprising an antigen recognition site. For the purposes of this disclosure, the term antibody also encompasses Fc fusion proteins containing immunoglobulin-derived, naturally occurring and/or synthetic amino acid sequences (e.g., peptibodies) that bind an expressed on a cell of interest to be targeted (e.g., cell surface immune checkpoint antigen such as PD-1L.)

In particular embodiments, the antibodies used according to the disclosed methods have reduced effector function. In some embodiments, the antibodies contain mutations in the Fc region responsible for effector function, such as, one or more mutations described in Int. Appl. Publ. Nos. WO09/100309, WO06/076594, WO06/053301, WO06/047350; and WO99/58572; U.S. Pat. Nos. 6,737,056 and 5,624,821, and U.S. Appl. Publ. Nos. US 2010/0166740 and 2006/0134709, the contents of each of which is herein incorporated by reference in its entirety. By “reduced effector function” is intended a reduction of a specific effector function such as, ADCC or CDC, in comparison to a control (for example a polypeptide with a wildtype Fc region), by at least 20%, at least 30% or by at least 50%.

A “blocking” antibody or an “antagonist” antibody or agent is one which inhibits or reduces biological activity of the antigen it binds, such as PD1, PD-L1, CTLA-4, B7.1, B7.2 and/or B7H2. In a certain embodiment blocking antibodies or antagonist antibodies substantially or completely inhibit the biological activity of the antigen. Desirably, the biological activity is reduced by at least 10%, 20%, 30%, 50%, 70%, 80%, 90%, 95%, or about 100%.

As used herein, the term “CEA” refers to the full-length carcinoembryonic antigen (CEACAM5; CEA; CD66e), protein which is approximately 702 amino acids (prior to removal of N- and C-terminal pro-sequences; N-terminal pro-sequence is approximately 34 amino acid signal sequence and C-terminal pro-sequence is approximately 17 amino acid sequence), the mature CEA sequence resulting after prosequence removal (GenBank at NCBI RefSeq NP 004354.2; SEQ ID NO:4) and the corresponding mature native CEA expressed on the surface of a cell. CEA is frequently expressed in carcinomas of the lung, pancreas, stomach, ovary, uterus, breast, colon and rectum (Hammarstrom S., Semin. Can. Biol. 9:67-81 (1999)). Cell lines expressing CEA such as, Ls174T (ATCC CCL-188, colon carcinoma), AsPC-1 (ATCC CRL-1682, pancreatic adenocarcinoma) are also known in the art. Note that the binding of a bispecific antibody or other composition disclosed herein to both soluble and membrane anchored mature target CEA is not considered herein to be binding to a non-target form of CEA, nor is it to be considered as evidence of lack of immunospecificity.

As used herein, the term “specifically binds” refers to the situation in which one member of a specific binding pair, such as an antibody, does not significantly bind to molecules other than its specific binding partner(s) (i.e., cross-reactivity of less than about 25%, 20%, 15%, 10%, or 5%) as measured by a technique in the art, at a diagnostically or therapeutically relevant concentration e.g., by competition ELISA or by measurement of KD with BIACORE or KINEXA assay. The term is applicable in instances where an agent such as a bispecific antibody, contains two or more binding portions that provide for the specific binding of distinct antigens or epitopes, in which case the agent is said to be able to specifically bind each antigen or epitope. The term specifically binds is also applicable where e.g., an antigen-binding portion of an antibody is specific for a particular epitope that is carried by a number of antigens, in which case the specific antibody carrying the antigen-binding domain will be able to specifically bind to the various antigens carrying the epitope. In certain embodiments, an antibody that specifically binds to CEA (i.e., CEACAM5) does not bind to carcinoembryonic antigen-related cell adhesion proteins such as, CEACAM1, CEACAM3, CEACAM4, CEACAM6, CEACAM7 and CEACAM8. In additional embodiments, an antibody that specifically binds human CEACAM5 having an amino acid sequence recited in SEQ ID NO:4 and one or more CEACAM5 variants having an amino acid sequence recited in SEQ ID NOS: 5, 6, or 7. In additional embodiments, an antibody that specifically binds to human CEACAM5 having an amino acid sequence recited in SEQ ID NO:4, but does not bind one or more human CEACAM5 variants having an amino acid sequence recited in SEQ ID NOS: 5, 6, or 7.

The term “BiTE”, when referring to a class of antibody or antibody-like molecules refers to bispecific T-cell engagers. Such molecules have a portion that is immunospecific for an antigen associated with a diseased state (e.g., an antigen expressed on cancerous cells) and a portion that links such a diseased cell to T cells. Additional exemplary description of BiTE type molecules are described in Int. Appl. Publ. Nos. WO13/012414, WO11/068758, WO09/070642, WO07/071426, WO05/061547, WO05/040220 and WO04/106380, and U.S. Pat. No. 8,394,926, the contents of each of which is herein incorporated by reference in its entirety.

As used herein, the term “MEDI-565” refers to a bispecific single chain antibody of the BiTE class that includes an anti-CEA binding portion and an anti-CD3 binding portion. MEDI-565 (contains a humanized anti-CEA (human CEACAM5) antibody and a deimmunized CD3c antibody connected by a short flexible linker sequence (Lutterbuese et al., J. Immunother., 32:341-352 (2009)). MEDI-565 is further described in Intl. Appl. Publ. No. WO07/071426, Lutterbuese et al., J. Immunother. 32:341-352 (2009), and Osada et al., Brit. J. Can., 102:124-33 (2010), the contents of each of which is herein incorporated by reference in its entirety. MEDI-565 and other CEA/CD3 BiTE antibodies have been reported to prevent subcutaneous tumor growth and formation of lung metastases in preclinical models (Lutterbuese et al., 2009). MEDI-565 has also been reported to inhibit proliferation of human CEA+ tumor cells and enhance T-cell redirected cytotoxicity of refractory metastatic primary colorectal cancer cells from patients previously treated with standard chemotherapy (including fluorouracil, oxaliplatin, and bevacizumab) in vitro (Osada et al., Brit. J. Cancer, 102:124-133 (2010), the contents of which is herein incorporated by reference in its entirety).

As used herein, the term MEDI4736 refers to an antibody having a light chain variable region comprising the amino acid sequence of SEQ ID NO:8 and a heavy chain variable region comprising the amino acid sequence of SEQ ID NO:9. MEDI4736 is further disclosed in Intl. Appl. Publ. No. WO 2011/066389 A1 and U.S. Appl. Publ. No. 2010/0028,330, the disclosure of each of which is herein incorporated by reference in its entirety. The Fc domain of MEDI4736 contains a triple mutation in the constant domain of the IgG1 heavy chain that reduces binding to the complement component Clq and the Fcγ receptors responsible for mediating antibody-dependent cell-mediated cytotoxicity (ADCC). MEDI4736 specifically binds PD-L1 (B7-H1) and blocks the binding of PD-L1 to the PD1 and CD80 (B7.1) receptors. MEDI4736 can relieve PD-L1-mediated suppression of human T-cell activation in vitro and inhibits tumor growth in a xenograft model via a T-cell dependent mechanism.

MEDI4736 and antigen-binding fragments thereof for use in the methods provided herein comprises a heavy chain and a light chain or a heavy chain variable region and a light chain variable region. In a specific embodiment, MEDI4736 or an antigen-binding fragment thereof for use in the methods provided herein comprises a light chain variable region comprising the amino acid sequence of SEQ ID NO:8 and a heavy chain variable region comprising the amino acid sequence of SEQ ID NO:9. In a particular embodiment, MEDI4736 or an antigen-binding fragment thereof for use in the methods provided herein comprises a heavy chain variable region and a light chain variable region, wherein the heavy chain variable region comprises the Kabat-defined CDR1, CDR2, and CDR3 sequences of SEQ ID NOS:10, 11 and 12, respectively, and wherein the light chain variable region comprises the Kabat-defined CDR1, CDR2, and CDR3 sequences of SEQ ID NOS:13, 14 and 15, respectively. Those of ordinary skill in the art would easily be able to identify Chothia-defined, Abm-defined or other CDR definitions known to those of ordinary skill in the art. In a specific embodiment, MEDI4736 or an antigen-binding fragment thereof for use in the methods provided herein comprises the variable heavy chain and variable light chain CDR sequences of the 2.14H9OPT antibody as disclosed in Intl. Appl. Publ. No. WO 2011/066389, the contents of which is herein incorporated by reference in its entirety.

The terms “antigen binding fragment” refers to a portion of an intact antibody and/or refers to the antigenic determining variable regions of an intact antibody. It is known that the antigen binding function of an antibody can be performed by fragments of a full-length antibody. Examples of antibody fragments include, but are not limited to, Fab, Fab′, F(ab′)2, and Fv fragments, linear antibodies, single chain antibodies, diabodies, and multispecific antibodies formed from antibody fragments.

As used herein, the term “immunoglobulin-like molecule” refers to an antibody mimic or antibody-like scaffold. In certain embodiments, immunoglobulin-like molecules may be any polypeptide comprising a non-immunoglobulin antigen binding scaffold, including, single chain antibodies, diabodies, minibodies, etc. Immunoglobulin-like molecules may contain an immunoglobulin-like fold. In certain embodiments, the immunoglobulin-like molecules may be derived from a reference protein by having a mutated amino acid sequence. In additional embodiments, the immunoglobulin-like molecule may be derived from an antibody substructure, minibody, adnectin, anticalin, affibody, knottin, glubody, C-type lectin-like domain protein, tetranectin, kunitz domain protein, thioredoxin, cytochrome b562, zinc finger scaffold, Staphylococcal nuclease scaffold, fibronectin or fibronectin dimer, tenascin, N-cadherin, E-cadherin, ICAM, titin, GCSF-receptor, cytokine receptor, glycosidase inhibitor, antibiotic chromoprotein, myelin membrane adhesion molecule P0, CD8, CD4, CD2, class I MHC, T-cell antigen receptor, CD1, C2 and I-set domains of VC AM-1,1-set immunoglobulin domain of myosin-binding protein C, 1-set immunoglobulin domain of myosin-binding protein H, I-set immunoglobulin domain of telokin, NCAM, twitchin, neuroglian, growth hormone receptor, erythropoietin receptor, prolactin receptor, interferon-gamma receptor, β-galactosidase/glucuronidase, β-glucuronidase, transglutaminase, T-cell antigen receptor, superoxide dismutase, tissue factor domain, cytochrome F, green fluorescent protein, GroEL, or thaumatin.

The term “subject” refers to any animal (e.g., a mammal), including, but not limited to, humans, non-human primates, rodents, and the like, which is to be the recipient of a particular treatment. Typically, the terms “subject” and “patient” are used interchangeably herein in reference to a human subject.

The term “pharmaceutical composition” refers to a preparation which is in such form as to permit the biological activity of the active ingredient (e.g., an immune checkpoint antagonist and a bispecific antibody disclosed herein) to be effective, and which contains no additional components which are unacceptably toxic to a subject to which the composition would be administered. Such composition can be sterile.

An “effective amount” of a composition used according to a method disclosed herein such as, immune checkpoint antagonists (ImCpAnts; e.g., an anti-PD1 antibody and an anti-PD-L1 antibody) and a bispecific antibody (e.g., CEA-BiTE)) is an amount sufficient to carry out a specifically stated purpose. An “effective amount” can be determined empirically and in a routine manner, in relation to the stated purpose. The term “effective amount” refers to a dosage or amount that is sufficient to result in amelioration of symptoms in a patient or to achieve a desired biological outcome, e.g., increased cytolytic activity of T cells, increased death of tumor cells, reduced tumor size, etc.

The term “therapeutically effective amount” refers to an amount of 1, 2, or more immune checkpoint antagonists and a multispecific T cell-redirecting agent disclosed herein or other drug effective to “treat” a disease or disorder (e.g., a tumor) in a subject or mammal. As used herein, the terms “treat”, “treatment” and “treating” in the context of administering a therapy or therapies to a patient refer to the reduction or amelioration of the progression, severity, and/or duration of an epithelial tumor. Said epithelial tumor(s) may be associated with aberrant expression e.g., overexpression or activity of CEA, and/or the amelioration of one or more symptoms thereof resulting from the administration of one or more therapies (including the administration of one or more pharmaceutical or therapeutic agents).

The terms “modulate” or “modulating an immune response” “immunomodulatory,” and their cognates refer to a reduction or an increase in the activity of inhibitory immune checkpoint pathway associated with upregulation of T cell responses due to its interaction of antagonists of the inhibitory pathway by for example ImCpAnts wherein the increase or decrease is relative to the antagonist target in the absence of the antagonist agent. An increase or a reduction in activity is preferably at least about 10%, 20%, 30%, 40%, 50%, 60%, 70%, 80%, 90%, or more. When the inhibitory activity of a target is decreased, the terms “modulatory” and “modulate” are interchangeable with the terms “activating” and “activate.” When the inhibitory activity of a target is increased, the terms “modulatory” and “modulate” are interchangeable with the terms “inhibitory” and “inhibit.” The activity of immune checkpoint targets can be determined quantitatively using T cell proliferation assays described herein or otherwise known in the art.

Terms such as “treating” or “treatment” or “to treat” refer to therapeutic measures that cure, slow down, lessen symptoms of, and/or halt progression of a diagnosed pathologic condition or disorder and also refer both therapeutic treatment and prophylactic/preventative measures. Thus, those in need of treatment include those already diagnosed with or suspected of having the disorder. Prophylactic or preventative measures refer to measures that prevent and/or slow the development of a targeted pathologic condition or disorder. Thus, those in need of prophylactic or preventative measures include those prone to have the disorder and those in whom the disorder is to be prevented.

### II. IMMUNE CHECKPOINT PATHWAYS

The terms “immune checkpoint”, “immune checkpoint receptor/ligand axis” and “immune checkpoint pathway” are used interchangeably herein to refer to a receptor/ligand signaling axis (pathway) that delivers negative signals in T cells and attenuate TCR-mediated signals. Under normal physiological conditions, immune checkpoints play crucial roles in maintaining self-tolerance and protecting tissues from damage during an immune response such as, a pathogen infection. Negative signals in T cells delivered by immune checkpoints may lead to for example, decreased cell proliferation, cytokine production, and/or cell cycle progression. Exemplary immune checkpoint pathways that can be targeted using the methods disclosed herein include, but are not limited to, the PD1/PD-L1 immune checkpoint pathway, and the cytotoxic T-lymphocyte antigen 4 (CTLA-4, CD152) immune checkpoint pathway.

Additional immune checkpoint pathways that can be targeted using the methods disclosed herein include, but are not limited to, an immune checkpoint pathway selected from: the BTLA (B- and T lymphocyte attenuator; also known as CD272), PDH1 (also known as V-domain Ig suppressor of T cell activation; VISTA), B7H3-TLT2 (also known as CD276), B7H4 (VCTN1), TIM3 (T cell immunoglobulin mucin 3; also known as HAVcr2), A2aR (adenosine A2a receptor), and/or the LAG3 (lymphocyte activation gene 3; also known as CD223) immune checkpoint pathway.

An antagonist composition that binds a receptor or ligand of an immune checkpoint pathway and attenuates signaling of the immune checkpoint pathway is referred to herein as an “immune checkpoint antagonists” (ImCpAnt).

### PD1/PD-L1 Immune Checkpoint Pathway

The PD1/PD-L1 immune checkpoint axis is believed to be involved in the maintenance of peripheral tolerance and to limit T cell effector functions within tissues. Disruption of PD1 expression has been reported to cause autoimmune disease like symptoms such as, a late-onset, progressive arthritis and lupus-like glomerulonephritis in mice. PD1 is expressed during thymic development primarily on CD4⁻CD8⁻ T cells, and induced on peripheral T cells, B cells, and monocytes upon activation. Members of the PD1/PD-L1 immune checkpoint pathway include for example, PD1, and the PD1 ligands PD-L1 (B7-H1, CD274) and PD-L2 (B7-DC, CD273). PD-L1 is expressed on lymphoid cells such as T and B cells as well as non-lymphoid organs including heart, liver, lung, pancreas, muscle, and placenta. In contrast, PD-L2 expression is restricted to DCs and macrophages.

In some embodiments, the disclosure provides the use of antagonists that specifically bind one, two, three or more members of the PD1/PD-L1 immune checkpoint pathway to practice the methods described herein. Thus in some embodiments, the methods use an antagonist such as a monoclonal antibody or an antigen binding fragment thereof, that specifically binds PD1, PD-L1 and/or PD-L2. Antagonists that specifically bind PD1, PD-L1 and/or PD-L2 are known and/or can be readily identified and prepared using techniques known in the art.

In further embodiments, a PD1 antagonist is used in practicing the methods disclosed herein. In some embodiments, the PD1 antagonist is an antibody or an antigen binding fragment thereof that binds PD1. In additional embodiments, the PD1 antagonist is an Fc fusion protein comprising an IgG Fc region fused to one or more polypeptides such as a portion of PD-L1 or PD-L2, an scFv, and a synthetic peptide that binds PD1.

In some embodiments, the PD1 antagonist competes with an antibody containing a VL having the sequence recited in SEQ ID NO:82 and a VH having the sequence recited in SEQ ID NO:83 for binding to PD1. In additional embodiments, the PD1 antagonist binds to the same epitope of PD1 as an antibody containing a VL having the sequence recited in SEQ ID NO:82 and a VH having the sequence recited in SEQ ID NO:83.

In some embodiments, the PD1 antagonist competes with an antibody containing a VL having the sequence recited in SEQ ID NO:84 and a VH having the sequence recited in SEQ ID NO:85 for binding to PD1. In additional embodiments, the PD1 antagonist binds to the same epitope of PD1 as an antibody containing a VL having the sequence recited in SEQ ID NO:84 and a VH having the sequence recited in SEQ ID NO:85.

In some embodiments, the PD1 antagonist competes with an antibody containing a VL having the sequence recited in SEQ ID NO:86 and a VH having the sequence recited in SEQ ID NO:87 for binding to PD1. In additional embodiments, the PD1 antagonist binds to the same epitope of PD1 as an antibody containing a VL having the sequence recited in SEQ ID NO:86 and a VH having the sequence recited in SEQ ID NO:87.

In some embodiments, the PD-1 antagonist competes with an antibody containing a VL having the sequence recited in SEQ ID NOS: 88-91 and a VH having the sequence recited in SEQ ID NOS:92-97 for binding to PD-1. In additional embodiments, the PD-1 antagonist binds to the same epitope of PD-1 as an antibody containing a VL having the sequence recited in any one of SEQ ID NOS:88-91 and a VH having the sequence recited in and one of SEQ ID NOS:92-97.

In additional embodiments, the PD1 antagonist competes with nivolumab (e.g., BMS-936558/MDX-1106/ONO-4538) for binding to PD1. In other embodiments, the PD1 antagonist binds to the same epitope of PD1 as nivolumab. In particular embodiments, the PD1 antagonist used according to the disclosed methods is nivolumab. See, e.g., Brahmer et al., J. Clin. Oncol. 28:3167-3175 (2010) and Topalian et al., N. Engl. J. Med. 28; 366 (26):2443-54 (2012).

In some embodiments, the PD1 antagonist competes with pidilizumab (e.g., CT-011; Curetech/Teva) for binding to PD1. In additional embodiments, the PD1 antagonist binds to the same epitope of PD1 as pidilizumab. In particular embodiments, the PD1 antagonist used according to the disclosed methods is pidilizumab. See, e.g., Berger et al., Clin. Cancer Res. 14:3044-3051 (2008).

In some embodiments, the PD1 antagonist competes with lambrolizumab (e.g., MK-3475; Merck) for binding to PD1. In additional embodiments, the PD1 antagonist binds to the same epitope of PD1 as lambrolizumab. In particular embodiments, the PD1 antagonist used according to the disclosed methods is lambrolizumab. See, e.g., Hamid et al., N. Engl. J. Med. 11369(2):134-44 (2013).

In some embodiments, a PD-L1 (B7H1) antagonist is used in practicing a method disclosed herein. In additional embodiments, the PD-L1 antagonist is an antibody or an antigen binding fragment thereof that binds PD-L1. In additional embodiments, the PD-L1 antagonist is an Fc fusion protein comprising an IgG Fc region fused to one or more polypeptides such as a portion of PD1, an scFv, or a synthetic peptide that binds PD-L1.

In some embodiments, the PD-L1 antagonist competes with an antibody containing a VL having the sequence recited in SEQ ID NO:62 and a VH having the sequence recited in SEQ ID NO:63 for binding to PD-L1. In additional embodiments, the PD-L1 antagonist binds to the same epitope of PD-L1 as an antibody containing a VL having the sequence recited in SEQ ID NO:62 and a VH having the sequence recited in SEQ ID NO:63.

In some embodiments, the PD-L1 antagonist competes with an antibody containing a VL having the sequence recited in SEQ ID NO:64 and a VH having the sequence recited in SEQ ID NO:65 for binding to PD-L1. In additional embodiments, the PD-L1 antagonist binds to the same epitope of PD-L1 as an antibody containing a VL having the sequence recited in SEQ ID NO:64 and a VH having the sequence recited in SEQ ID NO:65.

In some embodiments, the PD-L1 antagonist competes with an antibody containing a VL having the sequence recited in SEQ ID NO:66 and a VH having the sequence recited in SEQ ID NO:67 for binding to PD-L1. In additional embodiments, the PD-L1 antagonist binds to the same epitope of PD-L1 as an antibody containing a VL having the sequence recited in SEQ ID NO:66 and a VH having the sequence recited in SEQ ID NO:67.

In some embodiments, the PD-L1 antagonist competes with an antibody containing a VL having the sequence recited in SEQ ID NO:68 and a VH having the sequence recited in SEQ ID NO:69 for binding to PD-L1. In additional embodiments, the PD-L1 antagonist binds to the same epitope of PD-L1 as an antibody containing a VL having the sequence recited in SEQ ID NO:68 and a VH having the sequence recited in SEQ ID NO:69.

In some embodiments, the PD-L1 antagonist competes with an antibody containing a VL having the sequence recited in any one of SEQ ID NOS:70-75 and a VH having the sequence recited in any one of SEQ ID NOS:76-81 for binding to PD-L1. In additional embodiments, the PD-L1 antagonist binds to the same epitope of PD-L1 as an antibody containing a VL having the sequence recited in any one of SEQ ID NOS:70-75 and a VH having the sequence recited in any one of SEQ ID NOS:76-81.

In some embodiments, the PD-L1 antagonist competes with MEDI4736 (MedImmune/AstraZeneca) for binding to PD-L1. In additional embodiments, the PD-L1 antagonist binds to the same epitope of PD-L1 as MEDI4736. In particular embodiments, the PD-L1 antagonist used according to the disclosed methods is MEDI4736. See, e.g., U.S. Clinical Trial No: NCT01975831.

In additional embodiments, the PD-L1 antagonist competes with BMS-936559 (aka MDX-1105; Bristol-Myers Squibb) for binding to PD-L1. In additional embodiments, the PD-L1 antagonist binds to the same epitope of PD-L1 as BMS-936559. In particular embodiments, the PD-L1 antagonist used according to the disclosed methods is BMS-936559. See, e.g., Brahmer et al., N. Engl. J. Med. 366:2455-2465 (2012).

In additional embodiments, the PD-L1 antagonist competes with MPDL-3280A (aka RG7446, Genentech/Roche) for binding to PD-L1. In additional embodiments, the PD-L1 antagonist binds to the same epitope of PD-L1 as MPDL-3280A. In particular embodiments, the PD-L1 antagonist used according to the disclosed methods is MPDL-3280A. See, e.g., Chen, D., Ann Oncol. 24 (suppl 1): i7 (2013).

In some embodiments, a PD-L2 (B7 DC) antagonist is used in practicing a method disclosed herein. In some embodiments, the PD-L2 antagonist competes with rHIgM12B7 (Mayo Foundation) for binding to PD-L2. In additional embodiments, the PD-L2 antagonist binds to the same epitope of PD-L2 as rHIgM12B7. In particular embodiments, the PD-L2 antagonist used according to the disclosed methods is rHIgM12B7. See, e.g., U.S. Clinical Trial No: NCT00658892.

In some embodiments, the PD-L2 antagonist competes with AMP-224 (a B₇-DC/IgG1 fusion protein; Amplimmune/GlaxoSmithKline) for binding to PD-L2. In additional embodiments, the PD-L2 antagonist binds to the same epitope of PD-L2 as AMP-224. In particular embodiments, the PD-L2 antagonist used according to the disclosed methods is AMP-224. See, e.g., Smothers et al., Ann. Oncol. 24 (suppl. 1): i7 (2013).

In particular embodiments, a method disclosed herein uses a PD1 antagonist and a PD-L1 antagonist. In additional embodiments, a method disclosed herein uses a PD1 antagonist and a PD-L2 antagonist. In additional embodiments, a method disclosed herein uses a PD-L1 antagonist and a PD-L2 antagonist. In additional embodiments, a method disclosed herein uses a PD1 antagonist, a PD-L1 antagonist and a PD-L2 antagonist. In further embodiments, a method disclosed herein uses one or more of the above combinations wherein each antagonist is an antibody or an antigen binding fragment thereof that binds PD1, PD-L1 and/or PD-L2. In further embodiments, a method disclosed herein uses one or more of the above combinations wherein each antagonist is an antibody or an antigen binding fragment thereof that binds PD1, PD-L1 and/or PD-L2 in combination with a multispecific T cell-redirecting agent as described herein.

### CTLA4 Immune Checkpoint Pathway

The CTLA4 immune checkpoint pathway is believed to play a pivotal role in the regulation of autoreactive and potentially detrimental peripheral T cell responses. The disruption of CTLA4 expression has been reported to cause severe autoimmune phenotypes and death within 3-4 weeks of birth, in mice. CTLA4 is transcriptionally induced following T cell activation and is expressed in activated CD4⁺ T cells, CD8⁺ T cells, and Foxp3⁺ regulatory T cells (Tregs). Members of the CTLA4 immune checkpoint pathway include for example, CTLA-4, B7.1 (CD80), B7.2 (CD86), and B7H2 (ICOSL or CD275). B7.1, B7.2, and B7H2 are expressed on antigen presenting cells such as dendritic cells.

In some embodiments, the disclosure provides the use of antagonists that specifically bind one, two, three or more members of the CTLA4 immune checkpoint pathway to practice the methods described herein. Thus in some embodiments, the methods use an antagonist such as a monoclonal antibody or antigen binding fragment thereof, that specifically binds CTLA-4, B7.1, B7.2, and/or B7H2. Antagonists that specifically bind CTLA-4, B7.1, B7.2, and/or B7H2 are known and/or can be readily identified and prepared using techniques know in the art.

In some embodiments, a CTLA4 antagonist is used in practicing a method disclosed herein. In additional embodiments, the CTLA4 antagonist is an antibody or an antigen binding fragment thereof that binds CTLA4. In additional embodiments, the CTLA4 antagonist is an antibody that is an Fc fusion protein comprising an IgG Fc region fused to one or more polypeptides such as, a portion of B7.1, B7.2, or B7H2, an scFv, or a synthetic peptide that binds CTLA4.

In some embodiments, the CTLA4 antagonist competes with an anti-CTLA4 antibody disclosed in U.S. Pat. No. 6,682,736 (the disclosure of which is herein incorporated by reference in its entirety) for binding to CTLA4. In additional embodiments, the CTLA4 antagonist binds to the same epitope of CTLA4 as an anti-CTLA4 antibody disclosed in U.S. Pat. No. 6,682,736. In particular embodiments, the CTLA4 antagonist used according to the disclosed methods is an anti-CTLA4 antibody disclosed in U.S. Pat. No. 6,682,736.

In some embodiments, the CTLA4 antagonist competes with tremelimumab (Pfizer/AstraZeneca) for binding to CTLA4. In additional embodiments, the CTLA4 antagonist binds to the same epitope of CTLA4 as tremelimumab. In particular embodiments, the CTLA4 antagonist used according to the disclosed methods is tremelimumab. See, e.g., Ribas et al., The Oncologist 12:873-883 (2007); and Reuben et al., Cancer 106 (11):2437-2444 (2006).

In some embodiments, the CTLA4 antagonist competes with ipilimumab (e.g., YERVOY™; MDX-010, Bristol-Myers Squibb) for binding to CTLA-4. In additional embodiments, the CTLA4 antagonist binds to the same epitope of CTLA4 as ipilimumab. In particular embodiments, the CTLA4 antagonist used according to the disclosed methods is ipilimumab. See, e.g., Kaehler et al., Semin. Oncol. 37:485-498 (2010); Hodi et al., PNAS 100:4712-4717 (2003); Phan et al., PNAS 100:8372-8377 (2003).

In some embodiments, the CTLA4 antagonist competes with an antibody containing a VL having the sequence recited in SEQ ID NO:98 and a VH having the sequence recited in SEQ ID NO:99 for binding to CTLA4 antagonist. In additional embodiments, the CTLA4 antagonist binds to the same epitope of CTLA4 antagonist PD1 as an antibody containing a VL having the sequence recited in SEQ ID NO:98 and a VH having the sequence recited in SEQ ID NO:99. In further embodiments, the CTLA4 antagonist is an antibody containing a VL having the sequence recited in SEQ ID NO:98 and a VH having the sequence recited in SEQ ID NO:99.

In some embodiments, the CTLA4 antagonist competes with an antibody containing a VL having the sequence recited in SEQ ID NO:100 and a VH having the sequence recited in SEQ ID NO:101 for binding to CTLA4 antagonist. In additional embodiments, the CTLA4 antagonist binds to the same epitope of CTLA4 antagonist PD1 as an antibody containing a VL having the sequence recited in SEQ ID NO:100 and a VH having the sequence recited in SEQ ID NO:101. In further embodiments, the CTLA4 antagonist is an antibody containing a VL having the sequence recited in SEQ ID NO:100 and a VH having the sequence recited in SEQ ID NO:101.

In some embodiments, the CTLA4 antagonist competes with an antibody containing a VL having the sequence recited in SEQ ID NO:102 and a VH having the sequence recited in SEQ ID NO:103 for binding to CTLA4 antagonist. In additional embodiments, the CTLA4 antagonist binds to the same epitope of CTLA4 antagonist PD1 as an antibody containing a VL having the sequence recited in SEQ ID NO:102 and a VH having the sequence recited in SEQ ID NO:103. In further embodiments, the CTLA4 antagonist is an antibody containing a VL having the sequence recited in SEQ ID NO:102 and a VH having the sequence recited in SEQ ID NO:103.

In some embodiments, the CTLA4 antagonist competes with an antibody containing a VL having the sequence recited in SEQ ID NO:104 and a VH having the sequence recited in SEQ ID NO:105 for binding to CTLA4 antagonist. In additional embodiments, the CTLA4 antagonist binds to the same epitope of CTLA4 antagonist PD1 as an antibody containing a VL having the sequence recited in SEQ ID NO:104 and a VH having the sequence recited in SEQ ID NO:105. In further embodiments, the CTLA4 antagonist is an antibody containing a VL having the sequence recited in SEQ ID NO:104 and a VH having the sequence recited in SEQ ID NO:105.

In some embodiments, the CTLA4 antagonist competes with an antibody containing a VL having the sequence recited in SEQ ID NO:106 and a VH having the sequence recited in SEQ ID NO:107 for binding to CTLA4 antagonist. In additional embodiments, the CTLA4 antagonist binds to the same epitope of CTLA4 antagonist PD1 as an antibody containing a VL having the sequence recited in SEQ ID NO:106 and a VH having the sequence recited in SEQ ID NO:107. In further embodiments, the CTLA4 antagonist is an antibody containing a VL having the sequence recited in SEQ ID NO:106 and a VH having the sequence recited in SEQ ID NO:107.

In some embodiments, the CTLA4 antagonist competes with an antibody containing a VL having the sequence recited in SEQ ID NO:108 and a VH having the sequence recited in SEQ ID NO:109 for binding to CTLA4 antagonist. In additional embodiments, the CTLA4 antagonist binds to the same epitope of CTLA4 antagonist PD1 as an antibody containing a VL having the sequence recited in SEQ ID NO:108 and a VH having the sequence recited in SEQ ID NO:109. In further embodiments, the CTLA4 antagonist is an antibody containing a VL having the sequence recited in SEQ ID NO:108 and a VH having the sequence recited in SEQ ID NO:109.

In some embodiments, the CTLA4 antagonist competes with an antibody containing a VL having the sequence recited in SEQ ID NO:110 and a VH having the sequence recited in SEQ ID NO:111 for binding to CTLA4 antagonist. In additional embodiments, the CTLA4 antagonist binds to the same epitope of CTLA4 antagonist PD1 as an antibody containing a VL having the sequence recited in SEQ ID NO:110 and a VH having the sequence recited in SEQ ID NO:111. In further embodiments, the CTLA4 antagonist is an antibody containing a VL having the sequence recited in SEQ ID NO:110 and a VH having the sequence recited in SEQ ID NO:111.

In some embodiments, the CTLA4 antagonist competes with an antibody containing a VL having the sequence recited in SEQ ID NO:112 and a VH having the sequence recited in SEQ ID NO:113 for binding to CTLA4 antagonist. In additional embodiments, the CTLA4 antagonist binds to the same epitope of CTLA4 antagonist PD1 as an antibody containing a VL having the sequence recited in SEQ ID NO:112 and a VH having the sequence recited in SEQ ID NO:113. In further embodiments, the CTLA4 antagonist is an antibody containing a VL having the sequence recited in SEQ ID NO:112 and a VH having the sequence recited in SEQ ID NO:113.

In some embodiments, the CTLA4 antagonist competes with an antibody containing a VL having the sequence recited in SEQ ID NO:114 and a VH having the sequence recited in SEQ ID NO:115 for binding to CTLA4 antagonist. In additional embodiments, the CTLA4 antagonist binds to the same epitope of CTLA4 antagonist PD1 as an antibody containing a VL having the sequence recited in SEQ ID NO:114 and a VH having the sequence recited in SEQ ID NO:115. In further embodiments, the CTLA4 antagonist is an antibody containing a VL having the sequence recited in SEQ ID NO:114 and a VH having the sequence recited in SEQ ID NO:115.

In some embodiments, the CTLA4 antagonist competes with an antibody containing a VL having the sequence recited in SEQ ID NO:116 and a VH having the sequence recited in SEQ ID NO:117 for binding to CTLA4 antagonist. In additional embodiments, the CTLA4 antagonist binds to the same epitope of CTLA4 antagonist PD1 as an antibody containing a VL having the sequence recited in SEQ ID NO:116 and a VH having the sequence recited in SEQ ID NO:117. In further embodiments, the CTLA4 antagonist is an antibody containing a VL having the sequence recited in SEQ ID NO:116 and a VH having the sequence recited in SEQ ID NO:117.

In some embodiments, the CTLA4 antagonist competes with an antibody containing a VL having the sequence recited in SEQ ID NO:118 and a VH having the sequence recited in SEQ ID NO:119 for binding to CTLA4 antagonist. In additional embodiments, the CTLA4 antagonist binds to the same epitope of CTLA4 antagonist PD1 as an antibody containing a VL having the sequence recited in SEQ ID NO:118 and a VH having the sequence recited in SEQ ID NO:119. In further embodiments, the CTLA4 antagonist is an antibody containing a VL having the sequence recited in SEQ ID NO:118 and a VH having the sequence recited in SEQ ID NO:119.

In some embodiments, the CTLA4 antagonist competes with an antibody containing a VL having the sequence recited in SEQ ID NO:120 and a VH having the sequence recited in SEQ ID NO:121 for binding to CTLA4 antagonist. In additional embodiments, the CTLA4 antagonist binds to the same epitope of CTLA4 antagonist PD1 as an antibody containing a VL having the sequence recited in SEQ ID NO:120 and a VH having the sequence recited in SEQ ID NO:121. In further embodiments, the CTLA4 antagonist is an antibody containing a VL having the sequence recited in SEQ ID NO:120 and a VH having the sequence recited in SEQ ID NO:121.

In some embodiments, a B7.1 (CD80) antagonist is used in practicing a method disclosed herein. In additional embodiments, the B7.1 antagonist is an antibody or an antigen binding fragment thereof that binds B7.1. In additional embodiments, the B7.1 antagonist is an antibody that is an Fc fusion protein comprising an IgG Fc region fused to one or more polypeptides such as, a portion of B7.1, an scFv, or a synthetic peptide that binds B7.1.

In some embodiments, the B7.1 antagonist competes with galiximab (Biogen Idec) for binding to B7.1. In additional embodiments, the B7.1 antagonist binds to the same epitope of B7.1 as galiximab. In particular embodiments, the B7.1 antagonist used according to the disclosed methods is galiximab. See, e.g., Vinjamaram et al., Clin. Lymphoma Myeloma 8:277-282 (2008) and Czuczman et al., J. Clin. Oncol. 23:4390-4398 (2005).

In some embodiments, the B7.1 antagonist competes with IDEC-114 (Biogen Idec) for binding to B7.1. In additional embodiments, the B7.1 antagonist binds to the same epitope of B7.1 as IDEC-114. In particular embodiments, the B7.1 antagonist used according to the disclosed methods is IDEC-114. See, e.g., Schopf R., Curr Opin Investig. Drugs 2(5):635-8 (2001).

In some embodiments, a B7.2 (CD86) antagonist is used in practicing a method disclosed herein. In additional embodiments, the B7.2 antagonist is an antibody or an antigen binding fragment thereof that binds B7.2. In additional embodiments, the B7.2 antagonist is an antibody that is an Fc fusion protein comprising an IgG Fc region fused to one or more polypeptides such as, a portion of CTLA4 or CD28, an scFv, or a synthetic peptide that binds B7.2.

In some embodiments, a B7H2 (ICOSL or CD275) antagonist is used in practicing a method disclosed herein. In additional embodiments, the B7H2 antagonist is an antibody or an antigen binding fragment thereof that binds B7H2. In additional embodiments, the B7H2 antagonist is an antibody that is an Fc fusion protein comprising an IgG Fc region fused to one or more polypeptides such as, a portion of CTLA4 or CD28, an scFv, or a synthetic peptide that binds B7H2.

In some embodiments, the B7H2 antagonist competes with AMG-557 (Amgen) for binding to B7H2. In additional embodiments, the B7H2 antagonist binds to the same epitope of B7H2 as AMG-557. In particular embodiments, the B7H2 antagonist used according to the disclosed methods is AMG-557. See, e.g., U.S. Clinical Trial No: NCT00774943.

In particular embodiments, the methods disclosed herein use a CTLA4 antagonist and a B7.1 antagonist. In additional embodiments, the methods disclosed herein use a CTLA4 antagonist and a B7.2 antagonist. In additional embodiments, the methods disclosed herein use a CTLA4 antagonist and a B7H2 antagonist. In additional embodiments, the methods disclosed herein use a CTLA4 antagonist, a B7.1 antagonist, a B7.2 antagonist and a B7H2 antagonist. In additional embodiments, the methods disclosed herein use a CTLA4 antagonist and a B7.2 antagonist, a CTLA4 antagonist and a B7H2 antagonist, a B7.1 antagonist and a B7.2 antagonist, a B7.1 antagonist and a B7H2 antagonist, and/or a B7.2 antagonist and a B7H2 antagonist. In further embodiments, a method disclosed herein uses one or more of the above combinations wherein each antagonist is an antibody or an antigen binding fragment thereof that binds CTLA-4, B7.1, B7.2 and/or B7H2. In further embodiments, a method disclosed herein uses one or more of the above combinations wherein each antagonist is an antibody or an antigen binding fragment thereof that binds CTLA-4, B7.1, B7.2 and/or B7H2 in combination with a multispecific T cell-redirecting agent as described herein.

### BTLA Immune Checkpoint Pathway

The BTLA immune checkpoint pathway is believed to play an important role in the maintenance of immune tolerance and the prevention of autoimmune diseases. Disruption of BTLA expression has been reported to lead to enhanced T cell activation and exacerbated disease in mouse models of autoimmunity and inflammation. BTLA is expressed on T and B lymphocytes as well as subsets of DCs and is persistently expressed on tumor antigen-specific CD8⁺ T cells. Studies of peripheral blood mononuclear cells from patients with melanoma have reported that BTLA is expressed at high levels on tumor-specific CTLs and inhibits T cell function upon its engagement by tumor-expressed HVEM. BTLA binds HVEM (herpes virus entry mediator; TNFRSF14), which is an example of an additional antagonist target in the BTLA immune checkpoint pathway that may be antagonized according to the disclosed methods. HVEM is widely expressed in the hematopoietic system.

In some embodiments, the disclosure provides the use of antagonists that specifically bind one, two, three or more members of the BTLA immune checkpoint pathway to practice the methods described herein. Thus in some embodiments, the methods use an antagonist such as an antibody or antigen binding fragments thereof, that specifically binds BTLA and/or HVEM. Antagonists that specifically bind BTLA and/or HVEM can be readily identified and prepared using techniques know in the art.

In particular embodiments, the methods disclosed herein use a BTLA antagonist and a HVEM antagonist. In further embodiments, a BTLA antagonist and the HVEM antagonist are antibodies or antigen binding fragments thereof that bind BTLA and/or HVEM. In further embodiments, a method disclosed herein uses one or more of the above combinations wherein each antagonist is an antibody or an antigen binding fragment thereof that binds BTLA and/or HVEM in combination with a multispecific T cell-redirecting agent as described herein.

### B7H3 Immune Checkpoint Pathway

B7H3 is one of the most recently identified members of the B7/CD28 superfamily of costimulatory molecules that serves as an accessory modulator of T-cell response. B7H3 expression has been reported in several human cancers indicating an additional function of B7H3 as a regulator of antitumor immunity. However, its precise physiologic role is still elusive, because both stimulatory and inhibitory capacities have been demonstrated. B7H3 expression is inducible on T cells, NK cells and APCs and is upregulated on tumor cells, tumor-infiltrating cells, and endothelial cells of the tumor vasculature. B7-H3 is also broadly expressed on other cells of the body including, osteoblasts, fibroblasts, epithelial cells, as well as in liver, lung, bladder. Initial studies reported that B7-H3 enhanced the proliferation of both CD4⁺ and CD8⁺ T cells, the induction of cytotoxic T lymphocytes (CTLs) and the production of INF-γ.

In some embodiments, a B7H3 antagonist is used in practicing a method disclosed herein. In additional embodiments, the B7H3 antagonist is an antibody or an antigen binding fragment thereof that binds B7H3. In additional embodiments, the B7H3 antagonist is an antibody that is an Fc fusion protein comprising an IgG Fc region fused to one or more polypeptides such as, a portion of B7H3, an scFv, or a synthetic peptide that binds B7H3.

### B7H4 Immune Checkpoint Pathway

B7H4 (also known as VCTN1) is expressed in activated B cells, T cells, and monocytes, as well as in many human cancers such as, non small cell lung cancer, ovarian cancer, prostate cancer, breast cancer, and renal cancer. B7H4 pathway signaling suppresses T cell expansion, cytokine production, and arrests cell cycle at the G0/G1 phase. B7H4 signaling has been implicated in cancer progression is addition to its role in immune escape mechanisms. Antagonist anti-B7H4 antibodies have been reported to promote the growth of T cells and the secretion of cytokines such as, IL-2 and IFN-γ in vitro.

In some embodiments, a B7H4 antagonist is used in practicing a method disclosed herein. In additional embodiments, the B7H4 antagonist is an antibody or an antigen binding fragment thereof that binds B7H4. In additional embodiments, the B7H4 antagonist is an antibody that is an Fc fusion protein comprising an IgG Fc region fused to one or more polypeptides such as, a portion of B7H4, an scFv, or a synthetic peptide that binds B7H4.

### LAG3 Checkpoint Pathway

Lymphocyte activation gene 3 (LAG3; also known as CD223) is a co-inhibitory receptor that is broadly expressed in the haematopoietic system, including activated T cells and TReg cells, plasmacytoid Dendritic Cells, B cells, NK cells, NKT cells. LAG3 is coordinately upregulated on both TReg cells and anergic T cells. LAG3 signaling has been reported to inhibit T cell function in and LAG3 signaling has been implicated in tumor escape.

In some embodiments, a LAG3 antagonist is used in practicing a method disclosed herein. In additional embodiments, the LAG3 antagonist is an antibody or an antigen binding fragment thereof that binds LAG3. In additional embodiments, the LAG3 antagonist is an antibody that is an Fc fusion protein comprising an IgG Fc region fused to one or more polypeptides such as, a portion of LAG3, an scFv, or a synthetic peptide that binds LAG3.

### TIM3 Checkpoint Pathway

T cell immunoglobulin and mucin domain 3 (TIM3; also known as HAVcr2) is expressed on IFN-γ-secreting Th1 cells, Dendritic Cells (DCs), monocytes, CD8+ T cells, and other lymphocyte subsets. TIM3 is co-expressed with PD1 on tumor-specific CD8+ T cells. Binding of TIM3 by its ligand, galectin-9, results in Th1 cell death and blockade of TIM3 has been reported to increase IFN-γ-secreting T cells. Anti-TIM3 antibodies have been reported to enhance antitumor immunity. See, e.g., Ngiow et al., Cancer Res. 2011 Nov. 1; 71(21):6567-7. Galectin 9 is upregulated on various types of cancer, including breast cancers.

In some embodiments, a TIM3 antagonist is used in practicing a method disclosed herein. In additional embodiments, the TIM3 antagonist is an antibody or an antigen binding fragment thereof that binds TIM3. In additional embodiments, the TIM3 antagonist is an antibody that is an Fc fusion protein comprising an IgG Fc region fused to one or more polypeptides such as, a portion of TIM3 or galectin 9, an scFv, or a synthetic peptide that binds TIM3 or galectin 9.

### PD1H Checkpoint Pathway

PD1 homolog (PD1H, B7H5; also known as V-domain Ig suppressor of T cell activation; VISTA) is broadly expressed on hematopoietic cells and is upregulated on APC and T cells upon activation. VISTA is a co-inhibitory orphan ligand that has been reported to inhibit T cell proliferation and cytokine production by arresting cell cycle. VISTA signaling has been suggested to inhibit host protective antitumor immunity and VISTA expressed on cancer cells has been reported to diminish antitumor immunity and to enhance tumor-invasive growth in vitro.

In some embodiments, a VISTA antagonist is used in practicing a method disclosed herein. In additional embodiments, the VISTA antagonist is an antibody or an antigen binding fragment thereof that binds VISTA. In additional embodiments, the VISTA antagonist is an antibody that is an Fc fusion protein comprising an IgG Fc region fused to one or more polypeptides such as, a portion of VISTA, an scFv, or a synthetic peptide that binds VISTA.

### A2aR Checkpoint Pathway

A2aR signaling on macrophages, T cells, and dendritic cells has been shown to directly inhibit effector function. A2aR signaling inhibits T cell responses, in part by driving CD4+ T cells to express FOXP3 and hence to develop into TReg cells. The release of adenosine during tumor cell turnover is thought to produce a self-amplifying loop within the tumor that drives TReg production and accelerates tumor growth. A2aR engagement during activation has also been reported to promote T cell tolerance.

In some embodiments, a A2aR antagonist is used in practicing a method disclosed herein. In additional embodiments, the A2aR antagonist is an antibody or an antigen binding fragment thereof that binds A2aR. In additional embodiments, the A2aR antagonist is an antibody that is an Fc fusion protein comprising an IgG Fc region fused to one or more polypeptides such as, a portion of A2aR, an scFv, or a synthetic peptide that binds A2aR.

### III. IMMUNE ACTIVATING PATHWAYS

The terms “immune activating pathway” immune activating receptor/ligand axis” are used interchangeably herein to refer to a receptor/ligand signaling axis (pathway) that delivers signals in T cells and enhance or increase TCR-mediated signals. Enhanced or increased signals in T cells delivered by immune activating pathways may lead to for example, increased cell proliferation, cytokine production, and/or cell cycle progression. Exemplary immune activating pathways that can be targeted in some embodiments of the methods disclosed herein include, but are not limited to, the 4-1BB/CD137-CD137L, OX40-OX40L, GITRL-GITR, CD27-CD70, CD28-ICOS and/or the HVEM-LIGHT immune activating pathway.

An agonist composition that binds a receptor or ligand of an immune activating pathway and enhances signaling of the immune activating pathway is referred to herein as an “immune activating agonist” (ImActAg). In some embodiments, the disclosure provides the use of agonists that bind one, two, three or more ImActAgs of an immune activating pathway to practice the methods described herein. Thus in some embodiments, the methods use ImActAgs, such as monoclonal antibodies or antigen binding fragments thereof, that specifically bind one, two, three or more receptors or ligands in an immune activating pathway selected from the 4-1BB/CD137-CD137L, OX40-OX40L, GITRL-GITR, CD27-CD70, CD28-ICOS or the HVEM-LIGHT pathway. In additional embodiments, one, two, three or more ImActAgs is an antibody that is an Fc fusion protein comprising an IgG Fc region fused to one or more polypeptides such as, a portion of immune activating pathway protein, an scFv, or a synthetic peptide that binds an immune activating pathway protein.

In additional embodiments, the methods provide the use of ImActAgs, such as monoclonal antibodies, or antigen binding fragments thereof, that specifically bind one, two, three or more targets selected from 4-1BB/CD137, CD137L, OX40, OX40L, GITRL, GITR, CD27, CD70, CD28, ICOS, HVEM, and LIGHT. In some embodiments, the ImActAgs specifically bind two or more targets in an immune activating pathway. In some embodiments, the ImActAgs specifically bind two or more targets in different immune activating pathways. Antagonists that specifically bind 4-1BB/CD137, CD137L, OX40, OX40L, GITRL, GITR, CD27, CD70, CD28, ICOS, HVEM, and/or LIGHT are known and/or can be readily identified and prepared using techniques know in the art. In additional embodiments, one, two, three or more ImActAgs is an antibody that is an Fc fusion protein comprising an IgG Fc region fused to one or more polypeptides such as, a portion of immune activating pathway protein, an scFv, or a synthetic peptide that binds an immune activating pathway protein.

### 4-1BB/CD137-CD137L Immune Activating Pathway

4-1BB (CD137, also known as TNFRSF9) is an inducible costimulatory receptor expressed on activated CD4+ and CD8+ T cells, NKT cells, NK cells, DCs, macrophages, eosinophils, neutrophils, and mast cells. 4-1BB is typically constitutively expressed on APCs and Tregs. 4-1BB provides costimulatory signals to CD4+ and CD8+ T cells and also activates dendritic cells and other non-T-cells including monocytes, B cells, mast cells, NK cells, and neutrophils. 4-1BBL (also known as CD137 ligand (CD137L) and TNFSF9) is primarily expressed on APCs (DCs, B cells and macrophages) and is inducibly expressed on activated T cells and endothelial cells. Agonistic 4-1BB antibodies have been reported to induce antitumor activity in several animal models. This activity has been attributed to the enhanced activation of NK cells and CD8+ T-cells and the activation of the production of cytokines such as, IFN-γ.

In some embodiments, a 4-1BB agonist is used in practicing a method disclosed herein. In additional embodiments, the 4-1BB agonist is an antibody or an antigen binding fragment thereof that binds 4-1BB. In additional embodiments, the 4-1BB agonist is an antibody that is an Fc fusion protein comprising an IgG Fc region fused to one or more polypeptides such as, a portion of 4-1BB or 4-1BBL, an scFv, or a synthetic peptide that binds 4-1BB or 4-1BBL.

In some embodiments, the 4-1BB agonist competes with urelumab (e.g., BMS-663513; BMS) for binding to 4-1BB. In additional embodiments, the 4-1BB agonist binds to the same epitope of 4-1BB as urelumab. In particular embodiments, the 4-1BB agonist used according to the disclosed methods is urelumab. See, e.g., U.S. Clinical Trial No: NCT00774943.

### OX40-OX40L Immune Activation Pathway

OX-40 (also known as TNFRSF4) is an inducible costimulatory receptor expressed on activated CD4+ and CD8+ T cells as well as activated Tregs, NKT cells, NK cells, and neutrophils. OX-40L (also known as TNFSF4) expression is induced on APCs, as well as on T cells. OX-40 signaling amplifies T-cell responsiveness during T-cell/T-cell interactions and shares many functional similarities with CD137 signaling in the control of T cell activation, expansion, survival and memory T cell formation. OX-40 signaling inhibits Treg functions and counteracts the generation of inducible Tregs. Agonist OX40 antibodies have been reported to induce antitumor responses in cancer models for sarcoma, melanoma, colon carcinoma, and glioma.

In some embodiments, an OX40 agonist is used in practicing a method disclosed herein. In additional embodiments, the OX40 agonist is an antibody or an antigen binding fragment thereof that binds OX40. In additional embodiments, the OX40 agonist is an antibody that is an Fc fusion protein comprising an IgG Fc region fused to one or more polypeptides such as, a portion of OX40 or 4 OX40L, an scFv, or a synthetic peptide that binds OX40 or OX40L.

### GITRL-GITR Immune Activation Pathway

The costimulatory receptor GITR (Glucocorticoid-induced TNFR-related protein;

also known as TNFRSF18) is expressed on activated T cells and is constitutively, on Tregs. GITR ligand (GITRL, also known as TNFSF18) is constitutively expressed on peripheral tissues and is thought to engage GITR on tissue-infiltrating immune cells. GITR signaling has been reported to promote the proliferation of naïve T cells, cytokine production, and protection of T cells from activation-induced cell death. Agonist GITR antibodies have been reported to co-stimulate T cell proliferation and cytokine production in vitro, and to induce tumor regression in vivo in several tumor models through the activation of CD4+ T cells, CD8+ T cells and NK cells.

In some embodiments, a GITR agonist is used in practicing a method disclosed herein. In additional embodiments, the GITR agonist is an antibody or an antigen binding fragment thereof that binds GITR. In additional embodiments, the GITR agonist is an antibody that is an Fc fusion protein comprising an IgG Fc region fused to one or more polypeptides such as, a portion of GITR or GITRL, an scFv, or a synthetic peptide that binds GITR or GITRL.

In some embodiments, the GITR agonist competes with TRX518 for binding to GITR. In additional embodiments, the GITR agonist binds to the same epitope of GITR as TRX518. In particular embodiments, the GITR agonist used according to the disclosed methods is TRX518. See, e.g., U.S. Clinical Trial No: NCT01239134.

### CD27-CD70 Immune Activation Pathway

CD27 (also known as TNFRSF7) is expressed on naïve T and B cells, and NK cells. The expression of the CD27 ligand CD70 is restricted to APCs. CD27 signaling increases T-cell proliferation and survival. Agonist CD27 antibodies have been reported to expand tumor-specific CTLs and to induce antitumor activity in multiple animal models.

In some embodiments, a CD27 agonist is used in practicing a method disclosed herein. In additional embodiments, the CD27 agonist is an antibody or an antigen binding fragment thereof that binds CD27. In additional embodiments, the CD27 agonist is an antibody that is an Fc fusion protein comprising an IgG Fc region fused to one or more polypeptides such as, a portion of CD27 or CD70, an scFv, or a synthetic peptide that binds CD27 or CD70.

In some embodiments, the CD27 agonist competes with CDX-1127 for binding to CD27. In additional embodiments, the CD27 agonist binds to the same epitope of CD27 as CDX-1127. In particular embodiments, the CD27 agonist used according to the disclosed methods is CDX-1127. See, e.g., U.S. Clinical Trial No: NCT01460134.

### CD28-B7H2-ICOS Immune Activation Pathway

CD28 is constitutively expressed on naive T cells and provides the primary co-stimulatory signal to promote naive T cell priming following the engagement of B7.1, B7.2 or B7H2 which are expressed on APCs. CD28 signaling promotes T cell activation, differentiation and memory T cell formation. Inducible T-cell costimulator (ICOS/CD279) expressed on activated T cells, and like CD28, also binds B7H2. ICOS signaling regulates Th1 and Th2 cell differentiation and T cell effector responses. Agonist CD28 and ICOS antibodies have been reported to promote T cell proliferation and cytokine production in the presence of antigenic signals in vitro.

In some embodiments, a CD28 or ICOS agonist is used in practicing a method disclosed herein. In additional embodiments, the CD28 or ICOS agonist is an antibody or an antigen binding fragment thereof that binds CD28 or ICOS. In additional embodiments, the CD28 or ICOS agonist is an antibody that is an Fc fusion protein comprising an IgG Fc region fused to one or more polypeptides such as, a portion of CD28 or ICOS, an scFv, or a synthetic peptide that binds CD28 or ICOS.

### HVEM-LIGHT Immune Activation Pathway

Herpesvirus entry mediator (HVEM; also known as TNFRSF14) is widely expressed in the hematopoietic system. HVEM has been reported to bind BTLA, LIGHT (also known as TNFSF14) and LTα/TNF beta (also known as TNFSF1B). LIGHT expression has been reported on activated T cells, immature DCs, monocytes and NK cells and to be expressed on B cells following activation. Reports have suggested that the interaction between HVEM and LIGHT results in positive costimulatory signaling that induces T-cell proliferation and cytokine production. Agonist anti-HVEM antibodies have been reported to induce antitumor activity in animal models.

In some embodiments, a HVEM agonist is used in practicing a method disclosed herein. In additional embodiments, the HVEM agonist is an antibody or an antigen binding fragment thereof that binds HVEM. In additional embodiments, the HVEM agonist is an antibody that is an Fc fusion protein comprising an IgG Fc region fused to one or more polypeptides such as, a portion of HVEM or LIGHT, an scFv, or a synthetic peptide that binds HVEM or LIGHT.

### IV. MULTISPECIFIC T CELL-REDIRECTING AGENTS AND IMMUNE CHECKPOINT ANTAGONISTS

As used herein, the term “Multispecific T Cell-Redirecting agent” or “MsTC-Redir” refers to a single chain or multichain molecule containing two or more binding regions, wherein one of the binding regions specifically binds a cell surface antigen (such as a tumor associated antigen) on a target cell or tissue and wherein a second binding region of the molecule specifically binds an antigen (such as, CD3 or another activating receptor) on a T cell. This dual/multi-target binding ability of the MsTC-Redir recruits T cells and/or modulates T-cell mediated effector mechanisms that lead to the eradication of the targeted cell.

In some embodiments, the MsTC-Redir is a bispecific single chain antibody. Bispecific single chain molecules are known in the art and are described e.g., in Intl. Appl. Publ. No. WO 99/54440 or Mack, PNAS 92:7021-7025 (1995).

As used herein, a “bispecific single chain antibody” denotes a single polypeptide chain comprising two binding region. Each “binding domain” as used herein comprises one variable region from an antibody heavy chain (“VH region”), wherein the VH region of the first binding domain specifically binds to a first molecule (e.g., human CD3 molecule), and the VH region of the second binding domain specifically binds to a second molecule (e.g., a tumor associated antigen such as, human CEA (CEACAM5)). The two binding domains are optionally linked to one another by a short polypeptide spacer generally comprising on the order of 5 amino acids. Each binding domain may additionally comprise one variable region from an antibody light chain (“VL region”), the VH region and VL region within each of the first and second binding domains being linked to one another via a polypeptide linker, for example of the type disclosed and claimed in EP B1 623679, but in any case long enough to allow the VH region and VL region of the first binding domain and the VH region and VL region of the second binding domain to pair with one another such that, together, they are able to specifically bind to the respective first and second molecules. The arrangement of the V regions of the first or second binding domain may be VH-VL or VL-VH. In particular embodiments, the arrangement of the first binding domain specifically binds human CD3 and has a VH-VL arrangement. It is envisaged that the first binding domain may be located N-terminally or C-terminally to the second binding domain. Thus, exemplary arrangements of the binding domains of the bispecific single chain antibodies useful according to the method of the disclosure include for example, VH_{TAA}-VL_{TAA}-VH_{CD3}-VH_{CD3}, VL_{TAA}-VH_{TAA}-VH_{CD3}-VL_{CD3}, VH_{CD3}-VL_{CD3}-VH_{TAA}-VL_{TAA} or VH_{CD3}-VH_{CD3}-VL_{TAA}-VH_{TAA} (wherein TAA is a tumor associated antigen such as, CEA, EpCAM/TACSTD1, CD19, CD20, CD22, CD30, CD52, EGFR, ErbB2, ErbB3, MET, IGF-1R, PSA, PSMA, EpHA2, EpHA3, EpHA4, VEGF, VEGFR, folate binding protein, pgA33, TAG72, CAIX, CD33, ICOS, IL-5 receptor, integrin avb3, integrin a5b1, FAP, Tenascin, PD-L1 and MCSP).

In some embodiments, the disclosed methods use a bispecific single chain antibody wherein the first binding domain that specifically binds an activating receptor antigen on a T cell (e.g., CD3) is located C-terminally to the second binding domain. In further embodiments, the binding domains of the bispecific single chain antibodies are arranged in the order VH_{TAA}-VL_{TAA}-VH_{CD3}-VL_{CD3} or VL_{TAA}-VH_{TAA}-VH_{CD3}-VL_{CD3} (wherein TAA is a tumor associated antigen such as, CEA, EpCAM/TACSTD1, CD19, CD20, CD22, CD30, CD52, EGFR, ErbB2, ErbB3, MET, IGF-1R, PSA, PSMA, EpHA2, EpHA3, EpHA4, VEGF, VEGFR, folate binding protein, pgA33, TAG72, CAIX, CD33, ICOS, IL-5 receptor, integrin avb3, integrin a5b1, FAP, Tenascin, PD-L1 and MCSP, and wherein CD3 represents CD3 or another activating receptor expressed on a T cell).

In further embodiments, the disclosed methods use a bispecific single chain antibody wherein the first binding domain that specifically binds CD3 is located C-terminally to the second binding domain. And wherein the second binding domain specifically binds CEA (CEACAM5). In further embodiments, the binding domains of the bispecific single chain antibodies are arranged in the order VH_{CEA}-VL_{CEA}-VH_{CD3}-VH_{CD3} or VL_{CEA}-VH_{CEA}-VH_{CD3}-VL_{CD3}. In a particular embodiment, the bispecific single chain antibody construct has the amino acid sequence as set forth in SEQ ID NOs:3 or 16.

In some embodiments, the bispecific single chain antibody comprises (a) a first binding domain specifically binding to human CD3, and (b) a second binding domain specifically binding to human CEA, wherein said second binding domain comprises at least the amino acid sequence “DX₁X₂X₃X₄FYFDY” (SEQ ID NO:17), wherein “X₁”, “X₂”, “X₃” or “X₄” represents any amino acid residue, and the amino acid residue “D” corresponds to Kabat position 95 of CDR-H3 of murine monoclonal antibody A5B7 and the amino acid residues “FYFDY” correspond to Kabat positions 100, 100a, 100b, 101, and 102, respectively, of CDR-H3 of murine monoclonal antibody A5B7. In one embodiment, “X₁” represents “R” (Arginine), “F” (Phenylalanine), “M” (Methionine), “E” (Glutamic acid), or “T” (Threonine); “X₂” represents “G” (Glycine), “Y” (Tyrosine), “A” (Alanine), “D” (Aspartic acid), or “S” (Serine); “X₃” represents “L” (Leucine), “F” (Phenylalanine), “M” (Methionine), “E” (Glutamic acid), or “T” (Threonine); and “X₄” represents “R” (Arginine), “Y” (Tyrosine), “A” (Alanine), “D” (Aspartic acid), or “S” (Serine).

In additional embodiments, the CEA-binding domain of the bispecific antibody comprises the VH of SEQ ID NO:18 and the VL of SEQ ID NO:19, the VH-VL arrangement shown in SEQ ID NO:20, or the VL-VH arrangement of SEQ ID NO:21. In additional embodiments, the CEA-binding domain of the bispecific antibody comprises the VH of SEQ ID NO:22 and the VL of SEQ ID NO:19, the VH-VL arrangement shown in SEQ ID NO:23, or the VL-VH arrangement shown in SEQ ID NO:24. In additional embodiments, the CEA-binding domain of the bispecific antibody comprises the VH of SEQ ID NO:25 and the VL of SEQ ID NO:19, the VH-VL arrangement shown in SEQ ID NO:26, or the VL-VH arrangement depicted in SEQ ID NO:27.

Even more preferred, the V regions of the second binding domain specific for CEA of the bispecific single chain antibodies defined herein are selected from the group consisting of: (a) the VH region consists of the amino acid sequence shown in SEQ ID NO:28 and the VL region consists of the amino acid sequence shown in SEQ ID NO:19; (b) the VH region consists of the amino acid sequence shown in SEQ ID NO:29 and the VL region consists of the amino acid sequence shown in SEQ ID NO:19; (c) the VH region consists of the amino acid sequence shown in SEQ ID NO:18 and the VL region consists of the amino acid sequence shown in SEQ ID NO:19; (d) the VH region consists of the amino acid sequence shown in SEQ ID NO:22 and the VL region consists of the amino acid sequence shown in SEQ ID NO:19; and (e) the VH region consists of the amino acid sequence shown in SEQ ID NO:25 and the VL region consists of the amino acid sequence shown in SEQ ID NO:19.

Most preferred, said bispecific single chain antibody comprises an amino acid sequence selected from the group consisting of: (a) an amino acid sequence as depicted in any of SEQ ID NOS:3, 16, 30-43; (b) an amino acid sequence encoding by a nucleic acid sequence depicted in any of SEQ ID NOS:44-57; (c) an amino acid sequence encoded by a nucleic acid sequence hybridizing under stringent conditions to the complementary nucleic acid sequence of (b); (d) an amino acid sequence encoded by a nucleic acid sequence which is degenerate as a result of the genetic code to a nucleotide sequence of (b); and (e) an amino acid sequence at least 85% identical, at least 90% identical, at least 95% identical to the amino acid sequence of (a) or (b).

In some embodiments, the MsTC-Redir used according to the disclosed methods is a bispecific single chain antibody (e.g., bispecific single chain Fv (scFv)) with a deimmunized anti-CD3 binding domain (Int. Appl. Publ. No. WO 2005/040220) and a human anti-CEA binding domain comprising at least the amino acid sequence “FILNKANGGTTEKAAS” (SEQ ID NO:2).

In additional embodiments, the MsTC-Redir used according to the disclosed methods is a bispecific single chain antibody (e.g., bispecific single chain Fv (scFv)) with a deimmunized anti-CD3 binding domain (Int. Appl. Publ. No. WO 2005/040220) and a human anti-CEA binding domain comprising at least the amino acid sequence “DRGLRFYFDY” (SEQ ID NO:1). This sequence has been found to be sufficient to mediate resistance to soluble CEA when used in a human CEA-binding domain (i.e. a human binding domain specifically binding to human CEA) of CEA-BiTE. See, e.g., Intl. Publ. No. WO 2007 071426; Lutterbuese et al., J. Immunother. 32:341-352 (2009), and Osada et al., Br. J. Cancer 102:124-133 (2010), the contents of each of which are herein incorporated by reference in its entirety.

CEA levels in the blood of healthy individuals is less than 2 ng/ml. High soluble CEA concentrations in the serum/plasma of tumor patients are characteristic for progressive, recurrent, metastatic, or late stage tumors and for patients with high tumor load. In particular embodiments, wherein the MsTC-Redir composition used according to the disclosed methods binds human CEA, it is preferred that the cytotoxicity against CEA-positive target or tumor cells mediated by MsTC-Redir is not affected by increasing concentrations of soluble CEA. In particular, it is preferred that the cytotoxic activity of the MsTC-Redir is not inhibited by concentrations of soluble CEA (up to 10 ng/ml, 20 ng/ml, 30 ng/ml, up to 40 ng/ml, 50 ng/ml, 75 ng/ml, 100 ng/ml, 250 ng/ml, or up to 300 ng/ml). Thus, the compositions and methods disclosed herein are particularly advantageous for the treatment of patients with progressive tumors, metastasis, recurrent cancer, late stage epithelial tumors, high epithelial tumor load/tumor burden, or tumor patients with a CEA serum concentration higher than 100 ng/ml (as determined e.g., by ELISA), characterized by high levels of soluble CEA antigen in the plasma of the tumor patients.

As used herein, “human” refers to the species Homo sapiens. A “human” molecule, e.g., human CEA or human CD3 (CD3 epsilon), is therefore the variant of that molecule as it is naturally expressed in Homo sapiens.

The epithelial tumor to be treated using the methods disclosed herein may be a gastrointestinal adenocarcinoma, a breast adenocarcinoma or a lung adenocarcinoma. In some embodiments, the gastrointestinal adenocarcinoma is a colon, colorectal, pancreatic, esophageal or gastric adenocarcinoma. The epithelial tumors to be treated using the methods disclosed herein include, progressive tumors, recurrent cancer, metastatic tumors, high tumor load/burden, and late stage tumors. In particular embodiments the ImCpAnts and MsTC-Redir compositions are administered to treat progressive tumors, late stage tumors, tumor patients with high tumor load/burden, metastatic tumors, or tumor patients with a CEA serum concentration higher than 100 ng/ml.

It is also within the scope of the invention that the pharmaceutical compositions disclosed herein (e.g., ImCpAnts and MsTC-Redir compositions) be used after surgical removal of the primary tumor. For example, disseminated residual tumor cells derived from a CEA producing epithelial tumor also shed CEA into their microenvironments. Consequently, the tumor microenvironment and tissues surrounding these tumor cells often have high level of soluble CEA. Thus, it is preferred that the MsTC-Redir preferentially binds membrane-bound CEA over soluble CEA. The CEA serum concentration can be determined e.g., by CEA ELISA assays (see e.g., IBL CEA EIA, IBL Hamburg, Germany).

MsTC-Redir such as, bispecific antibodies and/or antigen binding fragments thereof that can bind tumor antigens and invariant epitopes of the CD3/TCR complex have been demonstrated to effectively recruit T lymphocytes to tumor targets in a non-cognate fashion, effectively bypassing the MHC context required for antigen recognition by the TCR.

As used herein, the term “CD3,” is used to refer individually or collectively to a molecule expressed as part of the T cell receptor and having a meaning as typically ascribed to it in the art. In humans, the term CD3 encompasses all known CD3 subunits, for example CD3 delta, CD3 epsilon, CD3 gamma, and CD3 zeta (TCR zeta), as well as CD3 alpha (TCR alpha), and CD3 beta (TCR beta) in individual or independently combined form. For example, in one embodiment, the MsTC-Redir binds the amino terminal 27 amino acids of mature CD3 epsilon. In particular embodiments, MsTC-Redir useful according to the disclosed methods binds invariant regions, or highly conserved regions of CD3. For example, in some embodiments, the MsTC-Redir specifically binds an invariant region of the TCR alpha beta subunits of the TCR. In further embodiments, the MsTC-Redir binds to the same epitope and/or competitively inhibits the binding of the murine BMA031 antibody to TCR. (See, e.g., Moore et al., Blood, 117, 17 (2011)).

In some embodiments, the MsTC-Redir specifically binds human CD3 epsilon. In additional embodiments, the MsTC-Redir specifically binds a human CD3 epsilon protein having the sequence of amino acids 23-207 set forth in NCBI Ref. Seq. No. NP_000724.

In additional embodiments, the MsTC-Redir specifically binds human CD3 epsilon and a CD3 epsilon ortholog from another organism. In some embodiments, the MsTC-Redir specifically binds human CD3 epsilon and a CD3 epsilon ortholog from a primate selected from a cynomolgus monkey, rhesus monkey, Saguinus oedipus, and Callithrix jacchus. In other embodiments, the MsTC-Redir specifically binds human CD3 epsilon and a CD3 epsilon ortholog from a mouse, rat, or a rabbit.

In another embodiment the MsTC-Redir specifically binds human CD3 delta. In further embodiments the MsTC-Redir specifically binds human CD3 delta having the sequence of amino acids 22-171 set forth in NCBI Ref. Seq. No. NP_000723. In additional embodiments, the MsTC-Redir specifically binds human CD3 delta and a CD3 delta ortholog from another organism. In some embodiments, the MsTC-Redir specifically binds human CD3 delta and a CD3 delta ortholog from a primate selected from a cynomolgus monkey, rhesus monkey, Saguinus oedipus, and Callithrix jacchus. In other embodiments, the MsTC-Redir specifically binds human CD3 delta and a CD3 delta ortholog from a mouse, rat, or a rabbit.

In an additional embodiment, the MsTC-Redir specifically binds human CD3 gamma. In further embodiments, the MsTC-Redir specifically binds human CD3 gamma having the sequence of amino acids 23-182 set forth in NCBI Ref. Seq. No. NP_000064. In additional embodiments, the MsTC-Redir specifically binds human CD3 gamma and a CD3 gamma ortholog from another organism. In some embodiments, the MsTC-Redir specifically binds human CD3 gamma and a CD3 gamma ortholog from a primate selected from a cynomolgus monkey, rhesus monkey, Saguinus oedipus, and Callithrix jacchus. In other embodiments, the MsTC-Redir specifically binds human CD3 gamma and a CD3 gamma ortholog from a mouse, rat, or a rabbit.

In an additional embodiment, the MsTC-Redir specifically binds a human CD3 zeta. In further embodiments, the MsTC-Redir specifically binds human CD3 zeta having the sequence of amino acids 22-164 set forth in NCBI Ref. Seq. No. NP_932170. In additional embodiments, the MsTC-Redir specifically binds human CD3 zeta and a CD3 zeta ortholog from another organism. In some embodiments, the MsTC-Redir specifically binds human CD3 zeta and a CD3 zeta ortholog from a primate selected from a cynomolgus monkey, rhesus monkey, Saguinus oedipus, and Callithrix jacchus. In other embodiments, the MsTC-Redir specifically binds human CD3 zeta and a CD3 zeta ortholog from a mouse, rat, or a rabbit.

The ImCpAnts and MsTC-Redir agents for use according to the disclosed methods include multispecific agents such as, bispecific antibodies. For example, MsTC-Redir agents used according to the claimed include bispecific antibodies that contain at least a first antigen binding site that specifically binds an antigen expressed on the surface of a cell to be targeted (e.g., a tumor associated antigen on a tumor cell) and a second antigen binding site that specifically binds an antigen expressed on the surface of an effector cell (e.g., a cytolytic T cell). Methods for making bispecific antibodies are known in the art. (See, e.g., Millstein et al., Nature, 305:537-539 (1983); Traunecker et al., EMBOJ., 10:3655-3659 (1991); Suresh et al., Methods in Enzymology, 121:210 (1986); Kostelny et al., J. Immunol. 148(5):1547-1553 (1992); Hollinger et al., PNAS USA, 90:6444-6448 (1993); Gruber et al., J. Immunol. 152:5368 (1994); U.S. Pat. Nos. 4,474,893; 4,714,681; 4,925,648; 5,573,920; 5,601,81; 95,731,168; 4,676,980; and 4,676,980, WO 94/04690; WO 91/00360; WO 92/200373; WO 93/17715; WO 92/08802; and EP 03089.)

Exemplary bispecific antibody molecules useful in practicing the methods described herein contain (i) two antibodies, a first antibody with a binding specificity to an antigen expressed on the surface of a tumor cell and a second antibody with a binding specificity for an antigen expressed on the surface of an effector cell (e.g., a cytolytic T cell) that are conjugated together, (ii) a single antibody that has one chain or arm with a binding specificity to an antigen expressed on the surface of a tumor cell and a second chain or arm with a binding specificity to an effector cell (e.g., a cytolytic T cell), (iii) a single chain antibody that has binding specificity to an antigen expressed on the surface of a tumor cell and also binding specificity to an effector cell (e.g., a cytolytic T cell), e.g., via two scFvs linked in tandem by an extra peptide linker; (iv) a dual-variable-domain antibody (DVD-Ig), where each light chain and heavy chain contains two variable domains in tandem through a short peptide linkage; (v) a chemically-linked bispecific (Fab′)₂ fragment; (vi) a Tandab, which is a fusion of two single chain diabodies resulting in a tetravalent bispecific antibody that has two binding sites for each of the target antigens; (vii) a flexibody (a combination of scFvs with a diabody resulting in a multivalent molecule); (viii) a so called “dock and lock” molecule (an adaptation of the “dimerization and docking domain” in Protein Kinase A, that can be applied to Fabs to generate a trivalent bispecific binding protein containing two identical Fab fragments linked to a different Fab fragment; (ix) a so-called “Scorpion” molecule, containing for example, two scFvs fused to both termini of a human Fc-region; (x) a diabody; and (xi) a so-called “ImmTAC” molecule (Immune mobilising mTCR Against Cancer; see e.g., Liddy et al., Nat. Med. 18:980-987 (2012)).

Both the ImCpAnts and MsTC-Redir compositions disclosed herein may be bispecific antibodies. Exemplary platforms for preparing bispecific antibodies for use according to the discloses methods include, but are not limited, to BiTE (Micromet), DART (MacroGenics), Fcab and Mab² (F-star), Fc-engineered IgG1 (Xencor) and DuoBody (Genmab).

Different classes of bispecific antibodies that can be used according to the disclosed methods include, but are not limited to, asymmetric IgG-like molecules (wherein one side of the molecule contains a Fab region or part of a Fab region of at least one antibody, and the other side of the molecule contains the Fab region or a part of a Fab region of at least one other antibody; symmetric IgG-like molecules (wherein the two sides of the molecule each contain the Fab region or part of the Fab region of at least two different antibodies; IgG fusion molecules, wherein full length IgG antibodies are fused to extra Fab regions or parts of Fab regions; Fc fusion molecules, wherein single chain Fv molecules or stabilized diabodies are fused to Fc gamma regions or parts thereof; Fab fusion molecules, wherein different Fab-fragments are fused together; and ScFv- and diabody-based molecules wherein different single chain Fv molecules or different diabodies are fused to each other or to another protein or carrier molecule.

Asymmetric IgG-like molecule platforms useful for bispecific antibodies used according to the disclosed methods include but are not limited to, the Triomab/Quadroma (Trion Pharma/Fresenius Biotech), Knobs-into-Holes (Genentech), CrossMAbs (Roche) electrostatically-matched (Amgen), LUZ-Y (Genentech), Strand Exchange Engineered Domain body (EMD Serono), Biclonic (Merus) and the DuoBody (Genmab A/S).

Symmetric IgG-like molecule platforms useful for bispecific antibodies used according to the disclosed methods include but are not limited to, Dual Targeting (DT)-Ig (GSK/Domantis), Two-in-one Antibody (Genentech), Cross-linked Mabs (Karmanos Cancer Center), mAb² (F-Star) and CovX-body (CovX/Pfizer).

IgG fusion molecule platforms useful for bispecific antibodies used according to the disclosed methods include but are not limited to, Dual Variable Domain (DVD)-Ig (Abbott), IgG-like Bispecific (ImClone/Eli Lilly), Ts2Ab (MedImmune/AZ), BsAb and zybody (Zymogenetics), HERCULES (Biogen Idec) and TvAb (Roche).

Fc fusion molecule platforms useful for bispecific antibodies used according to the disclosed methods include but are not limited to, ScFv/Fc Fusions (Academic Institution), SCORPION (Emergent BioSolutions/Trubion, Zymogenetics, BMS), Dual Affinity Retargeting Technology (Fc-DART) (MacroGenics) and Dual (ScFv)₂-Fab (Natl. Res. Cntr for Antibody Medicine—China).

Class V bispecific antibody molecule platforms useful for bispecific antibodies used according to the disclosed methods include but are not limited to, F(ab)₂ (Medarex/Amgen), Dual-Action or Bis-Fab (Genentech), Dock-and-Lock (DNL) (ImmunoMedics), Bivalent Bispecific (Biotecnol) and Fab-Fv (UCB-Celltech).

ScFv and diabody-based molecule platforms useful for bispecific antibodies used according to the disclosed methods include but are not limited to, Bispecific T Cell Engager (BiTE) (Micromet), Tandem Diabody (Tandab) (Affimed), Dual Affinity Retargeting Technology (DART) (MacroGenics), Single-chain Diabody (Academic), TCR-like Antibodies (AIT, ReceptorLogics), Human Serum Albumin ScFv Fusion (Merrimack) COMBODY (Epigen Biotech), and ImmTAC (Immune mobilising mTCR Against Cancer, Immunocore; see e.g., Liddy et al., Nat. Med. 18:980-987 (2012).

In some embodiments, the disclosure relates to pharmaceutical compositions containing one or more of the ImCpAnts and the MsTC-Redir (e.g., a bispecific antibody) for use according to the methods disclosed herein.

A preferred mode of administering the compositions for use according to the disclosed methods is an intravenous administration over a given time/time period. While bispecific single chain antibodies as described herein may be administered alone, preferred formulations of the co-administered ImCpAnts and MsTC-Redir are in a pharmaceutically acceptable carrier. Examples of suitable pharmaceutical carriers are known in the art and include phosphate buffered saline solutions, water, liposomes, various types of wetting agents, sterile solutions, etc. Compositions comprising such carriers can be formulated by conventional methods. These pharmaceutical compositions can be administered to the subject at a suitable dose. The dosage regimen will be determined by the attending physician and clinical factors. As is well known in the medical arts, dosages for any one patient depends upon many factors, including the patient's size, body surface area, age, the particular compound to be administered, sex, time and route of administration, general health, and other drugs being administered concurrently.

Preliminary doses as, for example, determined according to animal tests, and the scaling of dosages for human administration is performed according to art-accepted practices. Toxicity and therapeutic efficacy can be determined by standard pharmaceutical procedures in cell cultures or experimental animals. The data obtained from the cell culture assays or animal studies can be used in formulating a range of dosage for use in humans. Therapeutically effective dosages achieved in one animal model can be converted for use in another animal, including humans, using conversion factors known in the art (see, e.g., Freireich et al., Cancer Chemother. Reports, 50(4): 219-244 (1996)).

The disclosure provides compositions comprising ImCpAnts and/or the MsTC-Redir (e.g., a bispecific antibody). Such compositions may be suitable for pharmaceutical use and administration to patients. The compositions typically comprise one or more antibodies or antigen binding fragments thereof of the present invention and a pharmaceutically acceptable excipient. The phrase “pharmaceutically acceptable excipient” includes any and all solvents, dispersion media, coatings, antibacterial agents and antifungal agents, isotonic agents, and absorption delaying agents, and the like, that are compatible with pharmaceutical administration. The use of such media and agents for pharmaceutically active substances is well known in the art. The compositions may also contain other active compounds providing supplemental, additional, or enhanced therapeutic functions. The pharmaceutical compositions may also be included in a container, pack, or dispenser together with instructions for administration.

A pharmaceutical composition of the invention is formulated to be compatible with its intended route of administration. Methods to accomplish the administration are known to those of ordinary skill in the art. The administration may, for example, be intravenous, intraperitoneal, intramuscular, intracavity, subcutaneous or transdermal. It may also be possible to obtain compositions which may be topically or orally administered, or which may be capable of transmission across mucous membranes.

Solutions or suspensions used for intradermal or subcutaneous application typically include one or more of the following components: a sterile diluent such as water for injection, saline solution, fixed oils, polyethylene glycols, glycerin, propylene glycol, or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates; and agents for the adjustment of tonicity such as sodium chloride or dextrose. The pH can be adjusted with acids or bases, such as hydrochloric acid or sodium hydroxide. Such preparations may be enclosed in ampoules, disposable syringes or multiple dose vials made of glass or plastic.

Pharmaceutical compositions suitable for injection include sterile aqueous solutions or dispersions and sterile powders for the extemporaneous preparation of sterile injectable solutions or dispersion. For intravenous administration, suitable carriers include physiological saline, bacteriostatic water, Cremophor EL (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, and sodium chloride in the composition. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and/or by the use of surfactants. Prolonged absorption of the injectable compositions can be brought about by including in the composition an agent which delays absorption, for example, aluminum monostearate, and gelatin.

Oral compositions generally include an inert diluent or an edible carrier. They can be enclosed in gelatin capsules or compressed into tablets. For oral administration, the antibodies can be combined with excipients and used in the form of tablets, troches, or capsules. Pharmaceutically compatible binding agents, and/or adjuvant materials can be included as part of the composition. The tablets, pills, capsules, troches, and the like can contain any of the following ingredients, or compounds of a similar nature; a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

Systemic administration can also be by transmucosal or transdermal means. For transmucosal or transdermal administration, penetrants appropriate to the barrier to be permeated are used in the formulation. Such penetrants are generally known in the art, and include, for example, detergents, bile salts, and fusidic acid derivatives. Transmucosal administration may be accomplished, for example, through the use of lozenges, nasal sprays, inhalers or suppositories. For example, in case of antibodies that comprise the Fc portion, compositions may be capable of transmission across mucous membranes in intestine, mouth, or lungs (e.g., via the FcRn receptor-mediated pathway as described in U.S. Pat. No. 6,030,613). For transdermal administration, the active compounds may be formulated into ointments, salves, gels, or creams as generally known in the art. For administration by inhalation, the antibodies may be delivered in the form of an aerosol spray from pressured container or dispenser, which contains a suitable propellant, e.g., a gas such as carbon dioxide or a nebulizer.

In certain embodiments, the presently disclosed antibodies are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters and polylactic acid. Methods for preparation of such formulations will be apparent to those skilled in the art. Liposomal suspensions containing the presently disclosed antibodies can also be used as pharmaceutically acceptable carriers. These can be prepared according to methods known to those skilled in the art, for example, as described in U.S. Pat. No. 4,522,811.

It may be advantageous to formulate oral or parenteral compositions in a dosage unit form for ease of administration and uniformity of dosage. The term “dosage unit form” as used herein refers to physically discrete units suited as unitary dosages for the subject to be treated; each unit containing a predetermined quantity of active compound calculated to produce the desired therapeutic effect in association with the required pharmaceutical carrier.

Toxicity and therapeutic efficacy of the composition of the invention can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compositions that exhibit large therapeutic indices are preferred.

For any composition used in the present invention, the therapeutically effective dose can be estimated initially from cell culture assays. Examples of suitable bioassays include DNA replication assays, cytokine release assays, transcription-based assays, PD-1/PD-L1 binding assays, creatine kinase assays, assays based on the differentiation of pre-adipocytes, assays based on glucose uptake in adipocytes, immunological assays other assays as, for example, described in the Examples. The data obtained from the cell culture assays and animal studies can be used in formulating a range of dosage for use in humans. A dose may be formulated in animal models to achieve a circulating plasma concentration range that includes the IC₅₀ (i.e., the concentration of the antibody which achieves a half-maximal inhibition of symptoms). Circulating levels in plasma may be measured, for example, by high performance liquid chromatography. The effects of any particular dosage can be monitored by a suitable bioassay. The dosage lies preferably within a range of circulating concentrations with little or no toxicity. The dosage may vary depending upon the dosage form employed and the route of administration utilized.

A further aspect of the invention relates to the use of ImCpAnts and the MsTC-Redir (e.g., a bispecific single chain antibody) for the preparation of a pharmaceutical composition for the prevention, treatment or amelioration of a tumor such as, an epithelial tumor in a subject. Another aspect of the invention relates to a method for the prevention, treatment or amelioration of an epithelial tumor in a human, wherein the method comprises the step of administration of an effective amount of pharmaceutical compositions containing the ImCpAnts and MsTC-Redir of the invention. The person of ordinary skill in the art, in particular the attending physician can evaluate the successful treatment of the patient in need of administration of the pharmaceutical compositions containing the ImCpAnts and MsTC-Redir of the invention. Accordingly, the administration scheme as well as the dosage and the administration time may be assessed by said person skilled in the art: A corresponding “amelioration” and/or “treatment” to be assessed as defined herein.

In certain embodiments, the ImCpAnts and/or MsTC-Redir pharmaceutical compositions are administered in combination with one or more other therapies. In certain embodiments, the ImCpAnts and/or MsTC-Redir pharmaceutical compositions are administered to a patient concurrently with one or more other therapies. Preferably, such therapies are useful for the treatment of epithelial tumors. The term “concurrently” is not limited to the administration of pharmaceutical compositions or therapeutic agents at exactly the same time, but rather it is meant that the ImCpAnts and/or MsTC-Redir (e.g., a bispecific single chain antibody) pharmaceutical composition as defined herein and the other agent(s) are administered to a patient in a sequence and within a time interval such that the ImCpAnts and/or MsTC-Redir pharmaceutical compositions as defined herein can act together with the other agent to provide an increased benefit than if they were administered otherwise. For example, each therapeutic agent may be administered at the same time or sequentially in any order at different points in time; however, if not administered at the same time, they should be administered sufficiently close in time so as to provide the desired therapeutic effect. Each therapeutic agent can be administered separately, in any appropriate form and by any suitable route. In other embodiments, the ImCpAnts and/or MsTC-Redir pharmaceutical compositions as defined herein are administered before, concurrently or after surgery. Preferably the surgery completely removes localized epithelial tumors or reduces the size of large epithelial tumors. Surgery can also be done as a preventive measure or to relieve pain. The dosage amounts and frequencies of administration provided herein are encompassed by the term “therapeutically effective” as defined above. The dosage and frequency further will typically vary according to factors specific for each patient depending on the specific therapeutic or prophylactic agents administered, the severity and type of epithelial tumor, the route of administration, as well as age, body weight, response, and the past medical history of the patient. Suitable regimens can be selected by one skilled in the art by considering such factors and by following, for example, dosages reported in the literature and recommended in the Physicians' Desk Reference (59th ed., 2005).

In some embodiments, therapy by administration of the ImCpAnts and/or MsTC-Redir pharmaceutical composition as defined herein is combined with the administration of one or more therapies such as chemotherapies, radiation therapies, hormonal therapies, and/or biological therapies/immunotherapies. Therapeutic agents include, but are not limited to, proteinaceous molecules, including, but not limited to, peptides, polypeptides, proteins, including post-translationally modified proteins, antibodies etc.; or small molecules (less than 1000 Daltons), inorganic or organic compounds; or nucleic acid molecules including double-stranded or single-stranded DNA, or double-stranded or single-stranded RNA, as well as triple helix nucleic acid molecules. Therapeutic agents can be derived from any known organism (including, but not limited to, animals, plants, bacteria, fungi, and protista, or viruses) or from a library of synthetic molecules.

In a specific embodiment, the methods of the invention encompass administration of the ImCpAnts and MsTC-Redir (e.g., bispecific single chain antibody) pharmaceutical compositions as defined herein in combination with the administration of one or more therapeutic agents that are inhibitors of kinases such as gefitinib (IRESSA™), erlotinib (TARCEVA™), anti-EGFR-antibodies (e.g., cetuximab; ERBITUX), or anti-Her2/neu-antibodies (e.g., trastuzumab; HERCEPTIN™) described in the art; see e.g., Hardie and Hanks (1995) The Protein Kinase Facts Book, I and II, Academic Press, San Diego, Calif.

In another specific embodiment, the methods and uses of the invention encompass administration of the ImCpAnts and MsTC-Redir pharmaceutical compositions as defined herein in combination with the administration of one or more therapeutic agents that are angiogenesis inhibitors such as anti-VEGF-antibodies (e.g., bevacizumab; AVASTIN™), small molecular compounds (e.g., vatalanib or sorafenib) or COX-inhibitors.

In another specific embodiment, the methods and uses of the invention encompass administration of the ImCpAnts and MsTC-Redir pharmaceutical compositions as defined herein in combination with the administration of one or more therapeutic agents that are anti-cancer agents such as 5-Fluorouracil, leucovorin, capecitabine, lirinotecan, gemcitabine, doxorubicin, epirubicin, etoposide, cisplatin, carboplatin, taxanes (e.g., docetaxel, paclitaxel) described in the art.

In a particular specific embodiment, the methods and uses of the invention encompass the administration of 1, 2, or more ImCpAnts and/or MsTC-Redir in combination with the administration of an immunogenic chemotherapy. In further embodiments, the administered immunogenic chemotherapy is oxaliplatin.

Preferably, a co-therapy of a patient with an epithelial tumor using ImCpAnts and MsTC-Redir pharmaceutical compositions as defined herein in combination with (a) further therapeutic agent(s) results in an synergistic effect. As used herein, the term “synergistic” refers to a combination of therapies (e.g., a combination of ImCpAnts and/or MsTC-Redir as defined herein and (a) further therapeutic agent(s) as set forth above) which is more effective than the additive effects of any two or more single therapies (e.g., one or more therapeutic agents).

A synergistic effect of a combination of therapies (e.g., a combination of a ImCpAnts and MsTC-Redir as defined herein and (a) further therapeutic agent(s) as set forth above) permits the use of lower dosages of one or more of therapies (e.g., one or more therapeutic agents) and/or less frequent administration of said therapies to a patient with a disease, e.g., an epithelial tumor. The ability to utilize lower dosages of therapies (e.g., therapeutic agents) and/or to administer said therapies less frequently reduces the toxicity associated with the administration of said therapies to a subject without reducing the efficacy of said therapies in the prevention or treatment of a disease, e.g., an epithelial tumor. In addition, a synergistic effect can result in improved efficacy of therapies (e.g., therapeutic agents) in the prevention, management, treatment, amelioration or prevention of an epithelial tumor (which may be associated with aberrant expression (e.g., overexpression) or activity of CEA). Finally, synergistic effect of a combination of therapies (e.g., therapeutic agents) may avoid or reduce adverse or unwanted side effects associated with the use of any single therapy.

In co-therapy, an active agent may be optionally included in the same pharmaceutical composition as the ImCpAnts and MsTC-Redir pharmaceutical compositions defined herein, or may be included in a separate pharmaceutical composition. In this latter case, the separate pharmaceutical composition is suitable for administration prior to, simultaneously as or following administration of the pharmaceutical composition comprising ImCpAnts and/or MsTC-Redir (e.g., bispecific single chain antibody) as described herein. The additional drug or pharmaceutical composition may be a non-proteinaceous compound or a proteinaceous compound. In the case that the additional drug is a proteinaceous compound, is advantageous that the proteinaceous compound be capable of providing an activation signal for immune effector cells.

### V. METHODS OF MODULATING AND REDIRECTING IMMUNE RESPONSES USING ONE OR MORE IMCPANT AND MSTC-REDIR

The present disclosure provides methods for modulating and redirecting immune responses using (a) 1, 2, or more immune checkpoint antagonists (ImCpAnts) that specifically bind 2 or more different targets of an immune checkpoint pathway and (b) a multispecific T cell-redirecting agent (MsTC-Redir) that (i) specifically binds the cell surface antigen expressed on the surface of the cells of interest and (ii) specifically binds a T cell surface antigen. Also provided are methods of modulating and redirecting immune responses in a subject in immunotherapy (e.g., tumor therapy) using 1, 2, or more ImCpAnts and an MsTC-Redir.

The present disclosure also provides methods of killing cells of interest such as, tumor cells, diseased cells and infectious agents, using 1, 2, or more ImCpAnts and an MsTC-Redir. Additionally provided are methods of treating a tumor, disease or an infectious agent in a subject, comprising administering 1, 2, or more ImCpAnts and an MsTC-Redir to the subject.

In one embodiment, methods are provided for killing targeted cells in a cell population, comprising contacting a cell population containing target cells expressing a target associated antigen and T cells with (a) 1, 2, or more immune checkpoint antagonists that specifically bind 2 or more different targets of an immune checkpoint pathway and (b) a multispecific T cell-redirecting agent that (i) specifically binds the target associated antigen expressed on the target cells and (ii) specifically binds a T cell surface antigen, wherein the contacting of the cell population with (a) and (b) leads to death of target cells.

In some embodiments, the cell of interest is a tumor cell, an immune cell, a diseased cell or an infectious agent. Immune cells that can be targeted and killed using the methods described herein include B cell leukemia/lymphoma cells. Infectious agents that can be targeted and killed using the methods disclosed herein include, but are not limited to, prokaryotic and eukaryotic cells, viruses (including bacteriophage), foreign objects (e.g., toxins), and infectious organisms such as fungi, and parasites (e.g., mammalian parasites).

An additional embodiment provides a method of killing a tumor cell, comprising contacting a cell population containing tumor cells expressing a tumor associated antigen, and T cells, with (a) 1, 2, or more immune checkpoint antagonists (ImCpAnts) that specifically bind 2 or more different targets of targets of an immune checkpoint pathway and (b) a multispecific T cell-redirecting agent that (i) specifically binds the tumor associated antigen and (ii) specifically binds a T cell surface antigen, wherein the contacting of the cell population with (a) and (b) leads to death of tumor cells.

The present disclosure also provides a method of killing epithelial tumor cells, comprising contacting a cell population containing epithelial tumor cells expressing a tumor associated antigen, and T cells, with (a) 1, 2, or more immune checkpoint antagonists (ImCpAnts) that specifically bind 2 or more different targets of an immune checkpoint pathway, and (b) a multispecific T cell-redirecting agent that (i) specifically binds the epithelial tumor associated antigen and (ii) specifically binds a T cell surface antigen, wherein the contacting of the cell population with (a) and (b) leads to death of epithelial tumor cells.

In another embodiment, the disclosure provides a method of killing a CEA (CEACAM5) expressing tumor cell, comprising contacting a cell population containing tumor cells expressing CEA, and T cells with (a) 1, 2, or more immune checkpoint antagonists (ImCpAnts) that specifically bind 2 or more different targets of an immune checkpoint pathway, and (b) a multispecific T cell-redirecting agent that (i) specifically binds CEA and (ii) specifically binds a T cell surface antigen, wherein the contacting of the cell population with (a) and (b) leads to death of CEA expressing tumor cells.

In some embodiments, the tumor cell is an epithelial tumor cell or a cell from a cell line derived from an epithelial tumor. In further embodiments, the epithelial tumor cell, or derived cell, is from an epithelial tumor of the gastrointestinal tract. In further embodiments, the epithelial tumor cell, or derived cell, is from a gastrointestinal adenocarcinoma, a breast adenocarcinoma or a lung adenocarcinoma. In certain embodiments, the gastrointestinal adenocarcinoma is a colon, colorectal, pancreatic, an esophageal or a gastric adenocarcinoma. In additional embodiments, the epithelial tumor cell, or derived cell, is from a melanoma, renal cell carcinoma, non-small cell lung cancer, colon cancer, pancreatic cancer, esophageal cancer, gastric cancer or a colorectal cancer.

In additional embodiments, the epithelial tumor cell, or derived cell, is from a tumor or a cell culture previously contacted with a chemotherapeutic agent. In additional embodiments, the tumor or tumor cell is refractory to at least one chemotherapeutic agent. In further embodiments, the chemotherapeutic agent is vemurafenib, afatinib, cetuximab, carboplatin, bevacizumab, erlotinib, or pemetrexed.

In additional embodiments, one or more of the immune checkpoint antagonists (ImCpAnts) contacting the cell population according to the disclosed method is an antibody or an antigen binding fragment thereof. In some embodiments, the immune checkpoint pathway is the PD1-PD-L1 pathway, and/or the CTLA4 pathway. In further embodiments, one, two, three or more of the immune checkpoint antagonists (ImCpAnts) are antibodies or antigen-binding fragments thereof. In additional embodiments, the methods provide the use of ImCpAnts, that specifically bind one, two, three or more targets selected from PD1, PD-L1, PD-L2, CTLA4, B7.1, B7.2 and B7H2. In particular embodiments, an ImCpAnt used according to the disclosed method is an antagonist anti-PD1 antibody or an antigen-binding fragment thereof. In other embodiments, an ImCpAnt used according to the disclosed method is an antagonist anti-PD-L1 antibody or an antigen-binding fragment thereof. In further embodiments, ImCpAnts used according to the disclosed method are an antagonist anti-PD1 antibody or an antigen-binding fragment thereof and an antagonist anti-PD-L1 antibody or an antigen-binding fragment thereof. In yet further embodiments, ImCpAnts used according to the disclosed methods are an antagonist anti-PD1 antibody or an antigen-binding fragment thereof and an anti-PD-L2 antagonist antibody or an antigen-binding fragment thereof.

In additional embodiments the ImCpAnts used according to the disclosed methods include antagonists to two different targets on the PD1-PD-L1 immune checkpoint pathway and/or the CTLA4 pathway.

In some embodiments, the ImCpAnts include an antagonist anti-PD1 antibody and an antagonist anti-PD-L1 antibody. In additional embodiments, the ImCpAnts include 1, 2 or more of: (a) an anti-PD1 antibody or antigen binding fragment thereof, an anti-PD-L1 antibody or antigen binding fragment thereof, and/or an anti-PD-L2 antibody or antigen binding fragment thereof, and/or (b) an anti-CTLA4 antibody or antigen binding fragment thereof, an anti-B7.1 antibody or antigen binding fragment thereof, an anti-B7.2 antibody or antigen binding fragment thereof, and/or an anti-B7H2 antibody or antigen binding fragment thereof.

Additional immune checkpoint pathways that can be targeted using the methods disclosed herein include, but are not limited to, an immune checkpoint pathway selected from: the BTLA (B- and T lymphocyte attenuator; also known as CD272), PDH1 (also known as V-domain Ig suppressor of T cell activation; VISTA), B7H3-TLT2 (also known as CD276), B7H4 (VCTN1), TIM3 (T cell immunoglobulin mucin 3; also known as HAVcr2), A2aR (adenosine A2a receptor), and the LAG3 (lymphocyte activation gene 3; also known as CD223) immune checkpoint pathway. In additional embodiments, the methods provide the use of ImCpAnts, such as monoclonal antibodies, or antigen binding fragments thereof, that specifically bind one, two, three or more targets selected from BTLA, PDH1, B7H3, B7H4, TIM3, A2aR, and LAG3. In some embodiments, the ImCpAnts specifically bind two or more targets in an immune checkpoint pathway. In some embodiments, the ImCpAnts specifically bind two or more targets in different checkpoint pathways.

In some embodiments, the disclosed methods include the step of contacting the cell populations with agonists of one, two, three or more immune activating pathway (i.e., immune activating agonists (ImActAgs)). In some embodiments, the ImActAgs are monoclonal antibodies or antigen binding fragments thereof that specifically bind one, two, three or more receptors or ligands in an immune activating pathway selected from the 4-1BB/CD137-CD137L, OX40-OX40L, GITRL GITR, CD27-CD70, CD28-ICOS or the HVEM-LIGHT pathway. In additional embodiments, one, two, three or more ImActAgs is an antibody that is an Fc fusion protein comprising an IgG Fc region fused to one or more polypeptides such as, a portion of immune activating pathway protein, an scFv, or a synthetic peptide that binds an immune activating pathway protein. In additional embodiments, the disclosed methods include the step of contacting the cell populations with one, two, three or more ImActAgs that specifically bind one, two, three or more targets selected from 4-1BB/CD137, CD137L, OX40, OX40L, GITRL, GITR, CD27, CD70, CD28, ICOS, HVEM, and LIGHT. In some embodiments, the ImActAgs specifically bind two or more targets in an immune activating pathway. In some embodiments, the ImActAgs specifically bind two or more targets in different immune activating pathways.

In some embodiments, the cell population containing the tumor cells is contacted with 1, 2, or more ImCpAnts prior to being contacted with the multispecific T cell-redirecting agent (MsTC-Redir). In further embodiments, the cell population is contacted with 1, 2, or more ImCpAnts that specifically bind 2 or more different targets of an immune checkpoint pathway prior to being contacted with the multispecific T cell-redirecting agent (MsTC-Redir). In additional embodiments, the cell population containing the tumor cells is contacted with 1, 2, or more ImCpAnts at about the same time as (e.g., is co-administered or delivered within 1 hour of) the cell population is contacted with the MsTC-Redir. In further embodiments, the cell population is contacted with 1, 2, or more ImCpAnts that specifically bind 2 or more different targets of an immune checkpoint pathway at about the same time as (e.g., is co-administered or delivered within 1 hour of) the cell population is contacted with the MsTC-Redir. In additional embodiments, the cell population containing the tumor cells is contacted with 1, 2, or more ImCpAnts at least ½, 1, 2, 3, 4, 5, 6, 7, 8, 9, 10, 11, 12, 18, 24, 36, 48, 60 or 96 hours before the cell population is contacted with the MsTC-Redir. In further embodiments, the cell population is contacted with 1, 2, or more ImCpAnts that specifically bind 2 or more different targets of an immune checkpoint pathway at least ½, 1, 2, 3, 4, 5, 6, 7, 8, 9, 10, 11, 12, 18, 24, 36, 48, 60 or 96 hours before the cell population is contacted with the MsTC-Redir. In additional embodiments, the cell population is contacted with 1, 2, or more ImCpAnts at least ½ hour to 3 weeks, ½ hour to 2 weeks or ½ hour to 1 week before the cell population is contacted with the MsTC-Redir. In further embodiments, the cell population is contacted with 1, 2, or more ImCpAnts that specifically bind 2 or more different targets of an immune checkpoint pathway at least ½ hour to 3 weeks, ½ hour to 2 weeks or ½ hour to 1 week before the cell population is contacted with the MsTC-Redir. In additional embodiments, the cell population containing the tumor cells is contacted with 1, 2, or more ImCpAnts within ½, 1, 2, 3, 4, 5, 6, or 7 hours of the cell population being contacted with the MsTC-Redir. In particular embodiments, the cell population containing the tumor cells is contacted with 1, 2, or more ImCpAnts within, 6 hours of the cell population being contacted with the MsTC-Redir. In further embodiments, the cell population is contacted with 1, 2, or more ImCpAnts that specifically bind 2 or more different targets of an immune checkpoint pathway within ½, 1, 2, 3, 4, 5, 6, or 7 hours of the cell population being contacted with the MsTC-Redir. In particular embodiments, the cell population containing the tumor cells is contacted with 1, 2, or more ImCpAnts that specifically bind 2 or more different targets of an immune checkpoint pathway within, 6 hours of the cell population being contacted with the MsTC-Redir.

The contacting of the cell populations containing the targeted/tumor cells according to the disclosed methods can be in vitro, ex vivo, or in vivo.

In some embodiments, the MsTC-Redir used according to the disclosed methods is a bispecific antibody. In further embodiments, the bispecific antibody is selected from the group consisting of, a bispecific diabody, a single-chain bispecific diabody, a single chain bispecific tandem variable domain, a bispecific single domain antibody, a bispecific F(ab′)₂, a dock-and-lock bivalent or trivalent Fab, a bispecific (mab)₁, and a bispecific (mab)₂.

In additional embodiments, the bispecific antibody is a single-chain or a multichain bi-specific diabody. In particular embodiments, the bispecific diabody is a DART™ (MacroGenics). In other embodiments, the MsTC-Redir is a triabody (tribody) or a tetrabody. In other embodiments, the MsTC-Redir is a triomab, TandAb® or ImmTAC®.

In some embodiments, the MsTC-Redir specifically binds the CD3/TCR complex expressed on the surface of a T cell. In additional embodiments, the MsTC-Redir specifically binds and invariant region of the CD3/TCR complex and/or a member of the complex. In further embodiments the MsTC-Redir specifically binds an invariant region of the TCR alpha/beta subunits. In further embodiments, the MsTC-Redir specifically binds a CD3 target selected from CD3 delta, CD3 epsilon, CD3 gamma, CD3 zeta, TCR alpha, and TCR beta. In particular embodiments, the MsTC-Redir specifically binds a CD3 target selected from: CD3 delta having the sequence set forth in SEQ ID NO:59, CD3 epsilon having the sequence set forth in SEQ ID NO:58, CD3 gamma having the sequence set forth in SEQ ID NO:60, and CD3 zeta having the sequence set forth in SEQ ID NO:61.

In further embodiments the MsTC-Redir (e.g., a bispecific antibody, such as a BiTE) specifically binds CD3 epsilon. In further embodiments, the MsTC-Redir competes with CEA-BiTE for binding CD3 epsilon. In yet further embodiments, the MsTC-Redir binds to the same epitope of CD3 epsilon as CEA-BiTE (MEDI-565). In further embodiments, the MsTC-Redir contains the same CD3 epsilon binding sequence as CEA-BiTE. In further embodiments, the MsTC-Redir is CEA-BiTE (MEDI-565).

In additional embodiments, the MsTC-Redir used according to the disclosed methods is a bispecific antibody containing a single chain bispecific tandem variable domain (e.g., discFv). In particular embodiments, the MsTC-Redir is a bi-specific T-cell engager (BiTE). In further embodiments, the BiTE specifically binds the CD3/TCR complex expressed on the surface of a T cell. In additional embodiments, the BiTE specifically binds and invariant region of the CD3/TCR complex and/or a member of the complex.

In additional embodiments the MsTC-Redir (e.g., a bispecific antibody) binds CEA. In some embodiments, the MsTC-Redir competes with CEA-BiTE for binding with CEA. In additional embodiments, the MsTC-Redir binds to the same epitope of CEA as CEA-BiTE (MEDI-565). In further embodiments, the MsTC-Redir contains the same CEA binding sequence as CEA-BiTE (MEDI-565).

In another embodiment, the compositions and methods provide a method of modulating (e.g., increasing) and redirecting an immune response to a diseased cell or tissue and/or an immune cell in a subject, comprising, administering to the subject (a) 1, 2, or more immune checkpoint antagonists (ImCpAnts) that specifically bind 2 or more different targets of an immune checkpoint pathway and (b) a multispecific T cell-redirecting agent (MsTC-Redir) that (i) specifically binds an antigen on the surface of the diseased cell or tissue and/or an immune cell and (ii) specifically binds a T cell surface antigen.

In some embodiments, the cell, diseased cell or tissue is an immune cell, a tumor cell, or an infectious agent. In additional embodiments, the cell of interest is a tumor cell, an immune cell, a diseased cell or an infectious agent. Immune cells that can be targeted and killed using the methods described herein include B cell leukemia/lymphoma cells. Infectious agents that can be targeted and killed using the methods disclosed herein include, but are not limited to, prokaryotic and eukaryotic cells, viruses (including bacteriophage), foreign objects (e.g., toxins), and infectious organisms such as fungi, and parasites (e.g., mammalian parasites).

The disclosure also provides a method of treating a tumor in a subject, comprising administering to the subject (a) 1, 2, or more immune checkpoint antagonists (ImCpAnts) that specifically bind 2 or more different targets of an immune checkpoint pathway and (b) a multispecific T cell-redirecting agent (MsTC-Redir) that (i) specifically binds an antigen on the tumor cell surface and (ii) specifically binds a T cell surface antigen.

An additional embodiment provides a method of treating an epithelial tumor in a subject, comprising administering to the subject (a) 1, 2, or more immune checkpoint antagonists (ImCpAnts) that specifically bind 2 or more different targets of an immune checkpoint pathway, and (b) a multispecific T cell-redirecting agent that (i) specifically binds an epithelial tumor associated antigen and (ii) specifically binds a T cell surface antigen.

In some embodiments, the method provides a method of preventing an epithelial tumor in a subject, comprising administering to the subject (a) 1, 2, or more immune checkpoint antagonists (ImCpAnts) that specifically bind 2 or more different targets of an immune checkpoint pathway, and (b) a multispecific T cell-redirecting agent that (i) specifically binds an epithelial tumor associated antigen and (ii) specifically binds a T cell surface antigen, wherein the contacting of the cell population with (a) and (b) leads to death of CEA expressing tumor cells.

The term “preventing an epithelial tumor” as used herein is to be understood as follows: After surgical removal of the primary epithelial tumor(s) from a human patient and/or after chemotherapeutic or radiological treatment of the primary epithelial tumor(s), it may be the case that not all tumor cells could be eliminated from the body. However, these remaining tumor cells may give rise to recurrent cancer, i.e. local recurrence and/or metastases in the patient. Metastasis is a frequent complication of cancer, yet the process through which cancer cells disseminate from the primary tumor(s) to form distant colonies is poorly understood. Metastatic cancers are almost without exception incurable raising the necessity for new therapeutic modalities. The administration of pharmaceutical compositions containing ImCpAnts and/or MsTC-Redir according to the methods of the present disclosure can be used to kill these disseminated tumor cells in order to prevent the formation of secondary tumors (originating from the tumor cells remaining in the body after primary therapy). In this way, the administration of the pharmaceutical compositions helps to prevent the formation of local recurrence and/or metastases in tumor patients.

In another embodiment, the disclosure provides a method of treating a tumor containing cells expressing cell surface CEA in a subject, comprising administering to the subject (a) 1, 2, or more immune checkpoint antagonists (ImCpAnts) that specifically bind 2 or more different targets of an immune checkpoint pathway and (b) a multispecific T cell-redirecting agent that (i) specifically binds CEA and (ii) specifically binds a T cell surface antigen.

An additional embodiment provides a method of enhancing antitumor immunity in a subject comprising co-administering to a subject a bi-specific T-cell engager (BiTE) and two or more ImCpAnts. In some embodiments, the co-administered BiTE competes for binding with CD3 and/or CEA (CEACAM5) with an antibody or antigen binding fragment thereof comprising the amino acid sequence of SEQ ID NO:3. In additional embodiments the BiTE binds to the same epitope of CD3 and/or CEA (CEACAM5) as an antibody or antigen binding fragment thereof comprising the amino acid sequence of SEQ ID NO:3. In further embodiments, the BiTE is an antibody or antigen binding fragment thereof, comprising the amino acid sequence of SEQ ID NO:3.

An additional embodiment provides a method of reducing resistance of a tumor cell to T cell mediated killing comprising co-administering to a subject a bi-specific T-cell engager (BiTE) and two or more ImCpAnts. In some embodiments, the co-administered BiTE competes for binding with CD3 and/or CEA (CEACAM5) with an antibody or antigen binding fragment thereof comprising the amino acid sequence of SEQ ID NO:3. In additional embodiments the BiTE binds to the same epitope of CD3 and/or CEA (CEACAM5) as an antibody or antigen binding fragment thereof comprising the amino acid sequence of SEQ ID NO:3. In further embodiments, the BiTE is an antibody or antigen binding fragment thereof, comprising the amino acid sequence of SEQ ID NO:3.

An additional embodiment provides a method of enhancing antitumor immunity in a subject comprising co-administering to a subject a bi-specific T-cell engager (BiTE) and two or more ImCpAnts. In some embodiments, the co-administered BiTE competes for binding with CD3 and/or CEA (CEACAM5) with an antibody or antigen binding fragment thereof comprising the amino acid sequence of SEQ ID NO:16. In additional embodiments the BiTE binds to the same epitope of CD3 and/or CEA (CEACAM5) as an antibody or antigen binding fragment thereof comprising the amino acid sequence of SEQ ID NO:16. In further embodiments, the BiTE is an antibody or antigen binding fragment thereof, comprising the amino acid sequence of SEQ ID NO:16.

An additional embodiment provides a method of reducing resistance of a tumor cell to T cell mediated killing comprising co-administering to a subject a bi-specific T-cell engager (BiTE) and two or more ImCpAnts. In some embodiments, the co-administered BiTE competes for binding with CD3 and/or CEA (CEACAM5) with an antibody or antigen binding fragment thereof comprising the amino acid sequence of SEQ ID NO:16. In additional embodiments the BiTE binds to the same epitope of CD3 and/or CEA (CEACAM5) as an antibody or antigen binding fragment thereof comprising the amino acid sequence of SEQ ID NO:16. In further embodiments, the BiTE is an antibody or antigen binding fragment thereof, comprising the amino acid sequence of SEQ ID NO:16.

In some embodiments, the tumor in the subject treated according to the disclosed methods is an epithelial tumor. In further embodiments, the tumor is an epithelial tumor of the gastrointestinal tract. In further embodiments, the tumor is an gastrointestinal adenocarcinoma, a breast adenocarcinoma or a lung adenocarcinoma. In certain embodiments, the gastrointestinal adenocarcinoma is a colon, colorectal, pancreatic, an esophageal or a gastric adenocarcinoma. In additional embodiments, the epithelial tumor cell, or derived cell, is from a melanoma, renal cell carcinoma, non-small cell lung cancer, colon cancer, pancreatic cancer, esophageal cancer, gastric cancer or a colorectal cancer.

In additional embodiments, the subject treated according to the disclosed methods has previously been administered a chemotherapeutic agent. In additional embodiments, the tumor is refractory to at least one chemotherapeutic agent. In further embodiments, the chemotherapeutic agent is vemurafenib, erlotinib, afatinib, cetuximab, carboplatin, bevacizumab, or pemetrexed.

In additional embodiments, an immune checkpoint pathway targeted by the disclosed methods is the PD1-PD-L1 pathway, and/or the CTLA4 pathway. In further embodiments, 1, 2, 3 or more of the immune checkpoint antagonists (ImCpAnts) are antibodies or antigen-binding fragments thereof. In additional embodiments, the methods provide the use of ImCpAnts that specifically bind one, two, three or more targets selected from PD1, PD-L1, PD-L2, CTLA4, B7.1, B7.2 and B7H2. In particular embodiments, an ImCpAnt used according to the disclosed method is an antagonist anti-PD1 antibody or an antigen-binding fragment thereof. In other embodiments, an ImCpAnt used according to the disclosed method is an antagonist anti-PD-L1 antibody or an antigen-binding fragment thereof. In further embodiments, ImCpAnts used according to the disclosed method are an antagonist anti-PD1 antibody or an antigen-binding fragment thereof and an antagonist anti-PD-L1 antibody or an antigen-binding fragment thereof. In yet further embodiments, ImCpAnts used according to the disclosed methods are an antagonist anti-PD1 antibody or an antigen-binding fragment thereof and an anti-PD-L2 antagonist antibody or an antigen-binding fragment thereof.

In additional embodiments the ImCpAnts used according to the disclosed methods include antagonists to two different targets on the PD1-PD-L1 immune checkpoint pathway and/or the CTLA4 pathway.

In some embodiments, the ImCpAnts include an antagonist anti-PD1 antibody and an antagonist anti-PD-L1 antibody. In additional embodiments, the ImCpAnts include 1, 2 or more of: (a) an anti-PD1 antibody or antigen binding fragment thereof, an anti-PD-L1 antibody or antigen binding fragment thereof, and/or an anti-PD-L2 antibody or antigen binding fragment thereof, and/or (b) an anti-CTLA4 antibody or antigen binding fragment thereof, an anti-B7.1 antibody or antigen binding fragment thereof, an anti-B7.2 antibody or antigen binding fragment thereof, and/or an anti-B7H2 antibody or antigen binding fragment thereof.

Additional immune checkpoint pathways that can be targeted using the methods disclosed herein include, but are not limited to, an immune checkpoint pathway selected from: the BTLA (B- and T lymphocyte attenuator; also known as CD272), PDH1 (also known as V-domain Ig suppressor of T cell activation; VISTA), B7H3-TLT2 (also known as CD276), B7H4 (VCTN1), TIM3 (T cell immunoglobulin mucin 3; also known as HAVcr2), A2aR (adenosine A2a receptor), and the LAG3 (lymphocyte activation gene 3; also known as CD223) immune checkpoint pathway. In additional embodiments, the methods provide the use of ImCpAnts, such as monoclonal antibodies, or antigen binding fragments thereof, that specifically bind one, two, three or more targets selected from BTLA, PDH1, B7H3, B7H4, TIM3, A2aR, and LAG3. In some embodiments, the ImCpAnts specifically bind two or more targets in an immune checkpoint pathway. In some embodiments, the ImCpAnts specifically bind two or more targets in different checkpoint pathways.

In some embodiments, the disclosed methods include the step of administering agonists of one, two, three or more immune activating pathway (i.e., immune activating agonists (ImActAgs)). In some embodiments, the ImActAgs are monoclonal antibodies or antigen binding fragments thereof that specifically bind one, two, three or more receptors or ligands in an immune activating pathway selected from the 4-1BB/CD137-CD137L, OX40-OX40L, GITRL-GITR. CD27-CD70, CD28-ICOS or the HVEM-LIGHT. In additional embodiments, one, two, three or more ImActAgs is an antibody that is an Fc fusion protein comprising an IgG Fc region fused to one or more polypeptides such as, a portion of immune activating pathway protein, an scFv, or a synthetic peptide that binds an immune activating pathway protein. In additional embodiments, the disclosed methods include the step of administering one, two, three or more ImActAgs that specifically bind one, two, three or more targets selected from 4-1BB/CD137, CD137L, OX40, OX40L, GITRL, GITR, CD27, CD70, CD28, ICOS, HVEM, and LIGHT. In some embodiments, the ImActAgs specifically bind two or more targets in an immune activating pathway. In some embodiments, the ImActAgs specifically bind two or more targets in different immune activating pathways.

In further embodiments, the methods include administering an immune checkpoint antagonist to a subject determined to have a tumor expressing the immune checkpoint receptor/ligand to which the antagonist binds. Accordingly, in some embodiments, the methods further include the step of determining the expression of an immune checkpoint receptor or ligand in a tumor of a subject and administering to the subject (a) an immune checkpoint antagonist that specifically binds the immune checkpoint receptor or ligand and (b) a multispecific T cell-redirecting agent (MsTC-Redir) that (i) specifically binds an antigen on the tumor cell surface and (ii) specifically binds a T cell surface antigen. In some embodiments, the immune checkpoint receptor or ligand targeted by the administered ImCpAnt is determined to be expressed on tumor cells (e.g., cells in the region of a tumor having infiltrating lymphocytes). In some embodiments, the targeted immune checkpoint receptor or ligand is determined to be expressed on tumor infiltrating lymphocytes. In further embodiments, the targeted immune checkpoint receptor or ligand is determined to be expressed on antigen presenting cells (e.g., myeloid cells) in the tumor microenvironment. In particular embodiments, the immune checkpoint ligand is PD-L1. In further embodiments, the tumor expressing PD-L1 is a melanoma, renal cell carcinoma, non-small cell lung cancer, colon cancer, pancreatic cancer, esophageal cancer, gastric cancer, colorectal cancer, esophageal cancer, urothelial cancer, breast cancer or ovarian cancer. In additional embodiments, the immune checkpoint ligand is PD-L2. In further embodiments, the tumor expressing PD-L2 non-small cell lung cancer, pancreatic cancer, or ovarian cancer. In other embodiments, the immune checkpoint receptor is PD1. Methods for determining the expression of an immune checkpoint receptor or ligand in a biological sample such as, a tumor, are known in the art and can routinely be applied to practice the methods disclosed herein.

In some embodiments, the subject is administered 1, 2, or more ImCpAnts before the subject is administered the multispecific T cell-redirecting agent (MsTC-Redir). In further embodiments, the subject is administered 1, 2, or more ImCpAnts that specifically bind 2 or more different targets of an immune checkpoint pathway prior to being contacted with the multispecific T cell-redirecting agent (MsTC-Redir). In additional embodiments, the subject is administered 1, 2, or more ImCpAnts at about the same time as (e.g., is co-administered or delivered within 1 hour of) the cell population is contacted with the MsTC-Redir. In further embodiments, the subject is administered 1, 2, or more ImCpAnts that specifically bind 2 or more different targets of an immune checkpoint pathway at about the same time as (e.g., is co-administered or delivered within 1 hour of) the cell population is contacted with the MsTC-Redir. In additional embodiments, the c the subject is administered 1, 2, or more ImCpAnts at least ½, 1, 2, 3, 4, 5, 6, 7, 8, 9, 10, 11, 12, 18, 24, 36, 48, 60 or 96 hours before the subject is administered the MsTC-Redir. In further embodiments, the subject is administered 1, 2, or more ImCpAnts that specifically bind 2 or more different targets of an immune checkpoint pathway at least ½, 1, 2, 3, 4, 5, 6, 7, 8, 9, 10, 11, 12, 18, 24, 36, 48, 60 or 96 hours before the subject is administered the MsTC-Redir. In additional embodiments, the subject is administered 1, 2, or more ImCpAnts at least ½ hour to 3 weeks, ½ hour to 2 weeks or ½ hour to 1 week before the subject is administered the MsTC-Redir. In further embodiments, the subject is administered 1, 2, or more ImCpAnts that specifically bind 2 or more different targets of an immune checkpoint pathway at least ½ hour to 3 weeks, ½ hour to 2 weeks or ½ hour to 1 week before the subject is administered the MsTC-Redir. In additional embodiments, the subject is administered 1, 2, or more ImCpAnts within ½, 1, 2, 3, 4, 5, 6, or 7 hours of the subject being administered the MsTC-Redir. In particular embodiments, the subject is administered 1, 2, or more ImCpAnts within, 6 hours of the subject being administered the MsTC-Redir. In further embodiments, the subject is administered 1, 2, or more ImCpAnts that specifically bind 2 or more different targets of an immune checkpoint pathway within ½, 1, 2, 3, 4, 5, 6, or 7 hours of the subject being administered the MsTC-Redir. In particular embodiments, the subject is administered 1, 2, or more ImCpAnts that specifically bind 2 or more different targets of an immune checkpoint pathway within, 6 hours of the subject being administered the MsTC-Redir.

The administration of 1, 2, or more ImCpAnts and/or the MsTC-Redir may take place ex vivo or in vivo.

The success of the anti-tumor therapy may be monitored by established standard methods for the respective disease entities, e.g., by computer-aided tomography, X-ray, nuclear magnetic resonance tomography (e.g., for National Cancer Institute-criteria based response assessment, positron-emission tomography scanning, endoscopy, Fluorescence Activated Cell Sorting, aspiration of bone marrow, pleural or peritoneal fluid, tissue/histologies, and various epithelial tumor specific clinical chemistry parameters (e.g., soluble CEA concentration in serum) and other established standard methods may be used. In addition, assays determining T cell activation may be used; see e.g., WO99/054440. Statistics for the determination of overall survival, progression-free survival or relapse-free survival of treated tumor patients in comparison to non-treated tumor patients may also be used.

In some embodiments, the MsTC-Redir administered according to the disclosed methods is a bispecific antibody. In further embodiments, the bispecific antibody is selected from the group consisting of, a bispecific diabody, a single-chain bispecific diabody, a single chain bispecific tandem variable domain, a bispecific single domain antibody, a bispecific F(ab′)₂, a dock-and-lock bivalent or trivalent Fab, a bispecific (mab)₁, and a bispecific (mab)₂.

In additional embodiments, the bispecific antibody administered according to the disclosed methods is a single-chain or a multichain bi-specific diabody. In particular embodiments, the bispecific diabody is a DART™ (MacroGenics). In other embodiments, the MsTC-Redir is a triabody (tribody) or a tetrabody. In other embodiments, the MsTC-Redir is a triomab, TandAb® or ImmTAC®.

In some embodiments, the MsTC-Redir administered according to the disclosed methods specifically binds the CD3/TCR complex expressed on the surface of a T cell. In additional embodiments, the MsTC-Redir specifically binds and invariant region of the CD3/TCR complex and/or a member of the complex. In further embodiments the MsTC-Redir specifically binds an invariant region of the TCR alpha/beta subunits. In further embodiments, the MsTC-Redir specifically binds a CD3 target selected from CD3 delta, CD3 epsilon, CD3 gamma, CD3 zeta, TCR alpha, and TCR beta. In further embodiments, the MsTC-Redir specifically binds CD3 epsilon.

In further embodiments the MsTC-Redir (e.g., a bispecific antibody, such as a BiTE) specifically binds CD3 epsilon. In further embodiments, the MsTC-Redir competes with CEA-BiTE for binding CD3 epsilon. In yet further embodiments, the MsTC-Redir binds to the same epitope of CD3 epsilon as CEA-BiTE (MEDI-565). In further embodiments, the MsTC-Redir contains the same CD3 epsilon binding sequence as CEA-BiTE. In further embodiments, the MsTC-Redir is CEA-BiTE (MEDI-565).

In additional embodiments, the MsTC-Redir administered according to the disclosed methods is a bispecific antibody containing a single chain bispecific tandem variable domain (e.g., discFv). In particular embodiments, the MsTC-Redir is a bi-specific T-cell engager (BiTE). In further embodiments, the BiTE specifically binds the CD3/TCR complex expressed on the surface of a T cell. In additional embodiments, the BiTE specifically binds and invariant region of the CD3/TCR complex and/or a member of the complex.

In additional embodiments the MsTC-Redir (e.g., a bispecific antibody) administered according to the disclosed methods binds CEA. In some embodiments, the MsTC-Redir competes with CEA-BiTE for binding with CEA. In additional embodiments, the MsTC-Redir binds to the same epitope of CEA as CEA-BiTE (MEDI-565). In further embodiments, the MsTC-Redir contains the same CEA binding sequence as CEA-BiTE (MEDI-565).

### VI. PHARMACEUTICAL COMPOSITIONS AND ADMINISTRATION METHODS

Methods of preparing and administering immune checkpoint antagonists (ImCpAnts) and multispecific T cell-redirecting agents (MsTC-Redirs) to a subject in need thereof are well known to or are readily determined by those skilled in the art. The route of administration of the ImCpAnts and MsTC-Redir can be, e.g., oral, parenteral, by inhalation or topical. The term parenteral as used herein includes, e.g., intravenous, intraarterial, intraperitoneal, intramuscular, subcutaneous, rectal, or vaginal administration. However, in other methods compatible with the teachings herein, ImCpAnts and MsTC-Redir of the present disclosure can be delivered directly to the site of the targeted adverse cellular population (e.g., tumor) thereby increasing the exposure of the diseased tissue to the ImCpAnts and MsTC-Redir.

As discussed herein, ImCpAnts and MsTC-Redir can be administered in a pharmaceutically effective amount for the in vivo treatment of diseases mediated by the targeted cells specifically bound by the ImCpAnts and MsTC-Redir, such as cancer. The pharmaceutical compositions can comprise pharmaceutically acceptable carriers, including, e.g., water, ion exchangers, proteins, buffer substances, and salts. Preservatives and other additives can also be present. The carrier can be a solvent or dispersion medium. Suitable formulations for use in the therapeutic methods disclosed herein are described in Remington's Pharmaceutical Sciences (Mack Publishing Co.) 16th ed. (1980).

In any case, sterile injectable solutions can be prepared by incorporating an active compound (e.g., an ImCpAnt and an MsTC-Redir, by itself or in combination with other ImCpAnts, MsTC-Redir, or active agents) in the required amount in an appropriate solvent followed by filtered sterilization. Further, the preparations can be packaged and sold in the form of a kit. Such articles of manufacture can have labels or package inserts indicating that the associated compositions are useful for treating a subject suffering from, or predisposed to a disease or disorder.

Parenteral formulations can be a single bolus dose, an infusion or a loading bolus dose followed with a maintenance dose. These compositions can be administered at specific fixed or variable intervals, e.g., once a day, or on an “as needed” basis.

The composition can be administered as a single dose, multiple doses or over an established period of time in an infusion. Dosage regimens also can be adjusted to provide the optimum desired response (e.g., a therapeutic or prophylactic response).

Therapeutically effective doses of the compositions of the present disclosure, for treatment of diseases mediated by the targeted ImCpAnts and MsTC-Redir of the disclosure such as, cancer, vary depending upon many different factors, including means of administration, target site, physiological state of the patient, whether the patient is human or an animal, other medications administered, and whether treatment is prophylactic or therapeutic. Usually, the patient is a human, but non-human mammals including transgenic mammals can also be treated. Treatment dosages can be titrated using routine methods known to those of skill in the art to optimize safety and efficacy.

The amount of the ImCpAnts and MsTC-Redir to be administered can be readily determined by one of ordinary skill in the art without undue experimentation. Factors influencing the mode of administration and the respective amount of the ImCpAnts and MsTC-Redir include, but are not limited to, the severity of the disease, the history of the disease, and the age, height, weight, health, and physical condition of the individual undergoing therapy. Similarly, the amount of the ImCpAnts and MsTC-Redir to be administered will be dependent upon the mode of administration and whether the subject will undergo a single dose or multiple doses of each agent.

In additional embodiments, one, two three or more ImCpAnts are administered to a subject prophylactically prior to the subject being administered a cancer therapy. In some embodiments, one, two three or more ImCpAnts are administered to a subject prophylactically prior to the subject being administered other immunostimulatory therapy. In other embodiments, one, two three or more ImCpAnts are administered to a subject prophylactically prior to the subject being administered chemotherapy. In particular embodiments, the one, two or more ImCpAnts include an antagonist anti-PD1 antibody and an antagonist anti-PD-L1 antibody. In other embodiments, the one, two or more ImCpAnts include an antagonist anti-CTLA4 antibody and an antagonist anti-B.71, ant-B7.2 or an anti-B7H2 antibody

In additional embodiments, one, two three or more ImCpAnts are administered to a subject prophylactically prior to the subject being administered a vaccine. In particular embodiments, the one, two or more ImCpAnts include an antagonist anti-PD1 antibody and an antagonist anti-PD-L1 antibody.

The present disclosure also provides for the use of ImCpAnts and MsTC-Redir, in the manufacture of a medicament for treating a disease mediated by one or more of the cells specifically bound by one or more of the ImCpAnts and MsTC-Redir, such as cancer (tumors).

In some embodiments, the present disclosure provides for the co-administration of one, two, three or more ImCpAnts. One, two three or more ImCpAnts can be co-administered together in a single composition or can be co-administered together at the same time or overlapping times in separate compositions In additional embodiments, the immune checkpoint pathway is the PD1-PD-L1 pathway, and/or the CTLA4 pathway.

In further embodiments, one, two, three or more of the immune checkpoint antagonists (ImCpAnts) are antibodies or antigen-binding fragments thereof. In particular embodiments, an ImCpAnt used according to the disclosed method is an antagonist anti-PD1 antibody or an antigen-binding fragment thereof. In other embodiments, an ImCpAnt used according to the disclosed method is an antagonist anti-PD-L1 antibody or an antigen-binding fragment thereof. In further embodiments, ImCpAnts used according to the disclosed method are an antagonist anti-PD1 antibody or an antigen-binding fragment thereof and an antagonist anti-PD-L1 antibody or an antigen-binding fragment thereof. In yet further embodiments, ImCpAnts used according to the disclosed methods are an antagonist anti-PD1 antibody or an antigen-binding fragment thereof and an anti-PD-L2 antagonist antibody or an antigen-binding fragment thereof.

In additional embodiments the ImCpAnts used according to the disclosed methods include antagonists to two different targets on the PD1-PD-L1 immune checkpoint pathway and/or CTLA4 pathway. In particular embodiments, the ImCpAnts are a PD-L1 antagonist and a PD1 antagonist. In additional embodiments, the ImCpAnts include 1, 2 or more of: (a) an anti-PD1 antibody or antigen binding fragment thereof, an anti-PD-L1 antibody or antigen binding fragment thereof, and/or an anti-PD-L2 antibody or antigen binding fragment thereof, and/or (b) an anti-CTLA4 antibody or antigen binding fragment thereof, an anti-B7.1 antibody or antigen binding fragment thereof, an anti-B7.2 antibody or antigen binding fragment thereof, and/or an anti-B7H2 antibody or antigen binding fragment thereof.

In additional embodiments the ImCpAnts used according to the disclosed methods include antagonists to two different targets on the PD1-PD-L1 immune checkpoint pathway and/or CTLA4 pathway. In particular embodiments, the ImCpAnts are a PD-L1 antagonist and a PD1 antagonist. In additional embodiments, the ImCpAnts include 1, 2 or more of: (a) an anti-PD1 antibody or antigen binding fragment thereof, an anti-PD-L1 antibody or antigen binding fragment thereof, and/or an anti-PD-L2 antibody or antigen binding fragment thereof, and/or (b) an anti-CTLA4 antibody or antigen binding fragment thereof, an anti-B7.1 antibody or antigen binding fragment thereof, an anti-B7.2 antibody or antigen binding fragment thereof, and/or an anti-B7H2 antibody or antigen binding fragment thereof.

In additional embodiments, one, two three or more ImCpAnts are administered to a subject prophylactically prior to the subject being administered the MsTC-Redir. In some embodiments, the ImCpAnts and/or MsTC-Redir is a bispecific antibody. In further embodiments, the bispecific antibody is selected from the group consisting of, a bispecific diabody, a single-chain bispecific diabody, a single chain bispecific tandem variable domain, a bispecific single domain antibody, a bispecific F(ab′)₂, a dock-and-lock bivalent or trivalent Fab, a bispecific (mab)₁, and a bispecific (mab)₂. In particular embodiments, the MsTC-Redir is a bi-specific T-cell engager (BiTE). In further embodiments, the BiTE specifically binds the CD3/TCR complex expressed on the surface of a T cell. In further embodiments the MsTC-Redir competes with CEA-BiTE (MEDI-565) for binding with CD3 and/or CEA. In additional embodiments, the MsTC-Redir is CEA-BiTE. In particular embodiments, the one, two or more ImCpAnts include an antagonist anti-PD1 antibody and an antagonist anti-PD-L1 antibody. In additional embodiments, the one, two or more ImCpAnts include an antagonist anti-CTLA4 and an antagonist anti-B7.1, anti-B7.2, or anti-B7H2 antibody.

The present disclosure also provides for the co-administration of one or more ImCpAnts, MsTC-Redir and or additional therapy, each of which can be co-administered together at the same time or overlapping times in separate compositions.

In some embodiments, at least two doses of 1, 2, or more ImCpAnts and/or a MsTC-Redir (e.g., bispecific antibody) are administered to the patient. In some embodiments, at least three doses, at least four doses, at least five doses, at least six doses, at least seven doses, at least eight doses, at least nine doses, at least ten doses, or at least fifteen doses of any combination of the ImCpAnts and/or a MsTC-Redir can be administered to the patient. In some embodiments, 1, 2, or more ImCpAnts and/or a MsTC-Redir is administered over a two-week treatment period, over a four-week treatment period, over a six-week treatment period, over an eight-week treatment period, over a twelve-week treatment period, over a twenty-four-week treatment period, or over a one-year or more treatment period.

The amount of any give ImCpAnts and/or an MsTC-Redir to be administered to the patient will depend on various parameters such as the patient's age, weight, clinical assessment, tumor burden and/or other factors, including the judgment of the attending physician.

In additional embodiments, 1, 2, or more ImCpAnt antibodies or antigen binding fragments thereof are administered to the subject at a dose of about 0.1 mg/kg to about 15 mg/kg, about 0.3 mg/kg to about 10 mg/kg, or about 1 mg/kg to about 5 mg/kg. In some embodiments, the subject is administered 1, 2, or more ImCpAnts at a dose of about 0.1 mg/kg to about 15 mg/kg, about 0.3 mg/kg to about 10 mg/kg, or about 1 mg/kg to about 5 mg/kg. In further embodiments, the subject is administered 1, 2, 3 or more doses of 1, 2 or more ImCpAnt antibodies or antigen binding fragments thereof at about 1 week, 2 weeks, 3 weeks or more apart. In additional embodiments, the subject is administered 1, 2, 3 or more doses of 1, 2 or more ImCpAnt antibodies or antigen binding fragments thereof at about 1 day to 4 weeks, 1 week to 3 weeks, or 2 weeks to 3 weeks apart. In additional embodiments, the subject is administered 1, 2, 3 or more doses of 1, 2 or more ImCpAnt antibodies or antigen binding fragments thereof at about 1 week, 2 weeks, 3 weeks or more apart. In additional embodiments, the subject is administered 1, 2, 3, 4, or more doses of about 1 mg/kg to about 15 mg/kg, of 1, 2 or more ImCpAnt antibodies or antigen binding fragments thereof over a 2, 3 or 4 month period.

In some embodiments, the at least three doses are administered about two weeks apart. In some embodiment, the at least three doses are administered about three weeks apart.

In certain embodiments, administration of 1, 2 or more ImCpAnts and/or a MsTC-Redir according to the methods provided herein is through parenteral administration. For example, 1, 2 or more ImCpAnts and/or an MsTC-Redir can be administered by intravenous infusion or by subcutaneous injection. In some embodiments, the administration is by intravenous infusion.

In certain aspects, 1, 2 or more ImCpAnts and/or an MsTC-Redir is administered according to the methods provided herein in combination or in conjunction with additional cancer therapies. Such therapies include, without limitation, chemotherapeutic agents such as vemurafenib, afatinib, cetuximab, carboplatin, bevacizumab, erlotinib, or pemetrexed, or other chemotherapeutic agents, as well radiation or any other anti-cancer treatments.

The methods provided in many of the embodiments disclosed herein can decrease tumor size, retard tumor growth or maintain a steady state. In certain aspects the reduction in tumor size can be significant based on appropriate statistical analyses. A reduction in tumor size can be measured by comparison to the size of patient's tumor at baseline, against an expected tumor size, against an expected tumor size based on a large patient population, or against the tumor size of a control population. In certain aspects, use of the methods provided herein, i.e., administration of 1, 2 or more ImCpAnts and a MsTC-Redir can decrease tumor size within 6 weeks, within 7 weeks, within 8 weeks, within 9 weeks, within 10 weeks, within 12 weeks, within 16 weeks, within 20 weeks, within 24 weeks, within 28 weeks, within 32 weeks, within 36 weeks, within 40 weeks, within 44 weeks, within 48 weeks, or within 52 weeks of the first treatment.

In certain aspects, the patient has a particular type of tumor. In some embodiments, the tumor is a solid tumor. In some embodiments, the tumor is a melanoma, a renal cell carcinoma, a non-small cell lung cancer (e.g., squamous or adenocarcinoma), or a colorectal cancer. In some embodiments, the tumor is a melanoma, a non-small cell lung cancer (e.g., squamous or adenocarcinoma), or a colorectal cancer.

In some embodiments, the patient has previously received treatment with at least one chemotherapeutic agent. In some embodiments, the patient has previously received treatment with at least two chemotherapeutic agents. The chemotherapeutic agent can be, for example, and without limitation, vemurafenib, afatinib, cetuximab, carboplatin, bevacizumab, erlotinib, and/or pemetrexed.

In some embodiments, the tumor is refractory or resistant to at least one chemotherapeutic agent. In some embodiments, the tumor is refractory or resistant to at least two chemotherapeutic agents. The tumor can be refractory or resistant to one or more of, for example, and without limitation, vemurafenib, afatinib, cetuximab, carboplatin, bevacizumab, erlotinib, and/or pemetrexed.

Methods and reagents suitable for determination of binding characteristics of 2 or ImCpAnts and MsTC-Redir compositions of the invention are known in the art and/or are commercially available. Equipment and software designed for such kinetic analyses are commercially available (e.g., BIAcore, BIAevaluation software, GE Healthcare; KinExa Software, Sapidyne Instruments).

The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Sambrook et al., ed. (1989) Molecular Cloning A Laboratory Manual (2nd ed.; Cold Spring Harbor Laboratory Press); Sambrook et al., ed. (1992) Molecular Cloning: A Laboratory Manual, (Cold Springs Harbor Laboratory, NY); D. N. Glover ed., (1985) DNA Cloning, Volumes I and II; Gait, ed. (1984) Oligonucleotide Synthesis; Mullis et al.,U.S. Pat. No. 4,683,195; Hames and Higgins, eds. (1984) Nucleic Acid Hybridization; Hames and Higgins, eds. (1984) Transcription And Translation; Freshney (1987) Culture Of Animal Cells (Alan R. Liss, Inc.); Immobilized Cells And Enzymes (IRL Press) (1986); Perbal (1984) A Practical Guide To Molecular Cloning; the treatise, Methods In Enzymology (Academic Press, Inc., N.Y.); Miller and Calos eds. (1987) Gene Transfer Vectors For Mammalian Cells, (Cold Spring Harbor Laboratory); Wu et al., eds., Methods In Enzymology, Vols. 154 and 155; Mayer and Walker, eds. (1987) Immunochemical Methods In Cell And Molecular Biology (Academic Press, London); Weir and Blackwell, eds., (1986) Handbook Of Experimental Immunology, Volumes I-IV; Manipulating the Mouse Embryo, Cold Spring Harbor Laboratory Press, Cold Spring Harbor, N.Y., (1986); and in Ausubel et al., (1989) Current Protocols in Molecular Biology (John Wiley and Sons, Baltimore, Md.).

General principles of antibody engineering are set forth in Borrebaeck, ed. (1995) Antibody Engineering (2nd ed.; Oxford Univ. Press). General principles of protein engineering are set forth in Rickwood et al., eds. (1995) Protein Engineering, A Practical Approach (IRL Press at Oxford Univ. Press, Oxford, Eng.). General principles of antibodies and antibody-hapten binding are set forth in: Nisonoff (1984) Molecular Immunology (2nd ed.; Sinauer Associates, Sunderland, Mass.); and Steward (1984) Antibodies, Their Structure and Function (Chapman and Hall, New York, N.Y.). Additionally, standard methods in immunology known in the art and not specifically described are generally followed as in Current Protocols in Immunology, John Wiley & Sons, New York; Stites et al., eds. (1994) Basic and Clinical Immunology (8th ed; Appleton & Lange, Norwalk, Conn.) and Mishell and Shiigi (eds) (1980) Selected Methods in Cellular Immunology (W.H. Freeman and Co., NY).

Standard reference works setting forth general principles of immunology include Current Protocols in Immunology, John Wiley & Sons, New York; Klein (1982) J., Immunology: The Science of Self-Nonself Discrimination (John Wiley & Sons, NY); Kennett et al., eds. (1980) Monoclonal Antibodies, Hybridoma: A New Dimension in Biological Analyses (Plenum Press, NY); Campbell (1984) “Monoclonal Antibody Technology” in Laboratory Techniques in Biochemistry and Molecular Biology, ed. Burden et al., (Elsevere, Amsterdam); Goldsby et al., eds. (2000) Kuby Immunnology (4th ed.; H. Freemand & Co.); Roitt et al., (2001) Immunology (6th ed.; London: Mosby); Abbas et al., (2005) Cellular and Molecular Immunology (5th ed.; Elsevier Health Sciences Division); Kontermann and Dubel (2001) Antibody Engineering (Springer Verlan); Sambrook and Russell (2001) Molecular Cloning: A Laboratory Manual (Cold Spring Harbor Press); Lewin (2003) Genes VIII (Prentice Hall 2003); Harlow and Lane (1988) Antibodies: A Laboratory Manual (Cold Spring Harbor Press); Dieffenbach and Dveksler (2003) PCR Primer (Cold Spring Harbor Press).

All of the references cited above, as well as all references cited herein, are incorporated herein by reference in their entireties.

The following Examples do not in any way limit the scope of the invention. One of ordinary skill in the art will recognize the numerous modifications and variations that may be performed without altering the spirit or scope of the present invention. Such modifications and variations are encompassed within the scope of the invention. The entire contents of all references, patents, and published patent applications cited throughout this application are herein incorporated by reference.

### EXAMPLES

### Example 1

Early blockade of the PD1/PD-L1 pathway maximizes CEA/CD3-bispecific T-cell-engaging (BiTE) antibody-mediated cytotoxicity.

Recently, expression of PD-L1 by tumors has been shown to modulate function of activated T cells expressing PD1. Therefore, we explored whether T-cell exhaustion was observed with repeated MEDI-565 exposure and if so, by what mechanism. Furthermore, we assessed the effect of the PD1/PD-L1 immune checkpoint on T cell cytotoxicity. Finally, we attempted to restore T-cell cytolytic activity after previous MEDI-565 mediated attack with anti-PD1 and anti-PD-L1 antibodies.

The carcinoembryonic antigen (CEA)/CD3-bispecific T-cell-engaging (BiTE) antibody MEDI-565 (a CEA-BiTE, aka MT111 and AMG 211) simultaneously binds to T cells via CD3 and to tumor cells via CEA. We performed serial co-cultures of tumor cells with human T cells in the presence of CEA-BiTE. This enabled the study of PD1 and PD-L1 blockade and its effect on T cell survival and T cell-mediated killing of the CEA-expressing human cell lines SW1463 (colorectal) and AsPC-1 (pancreatic).

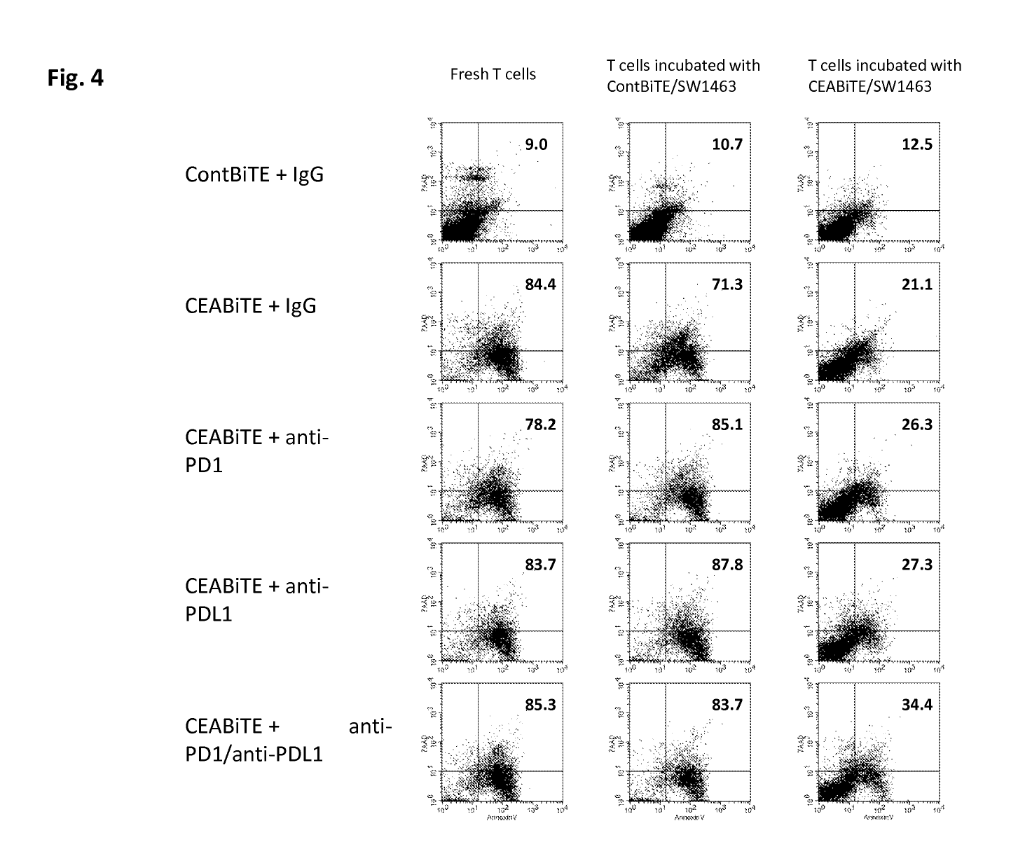

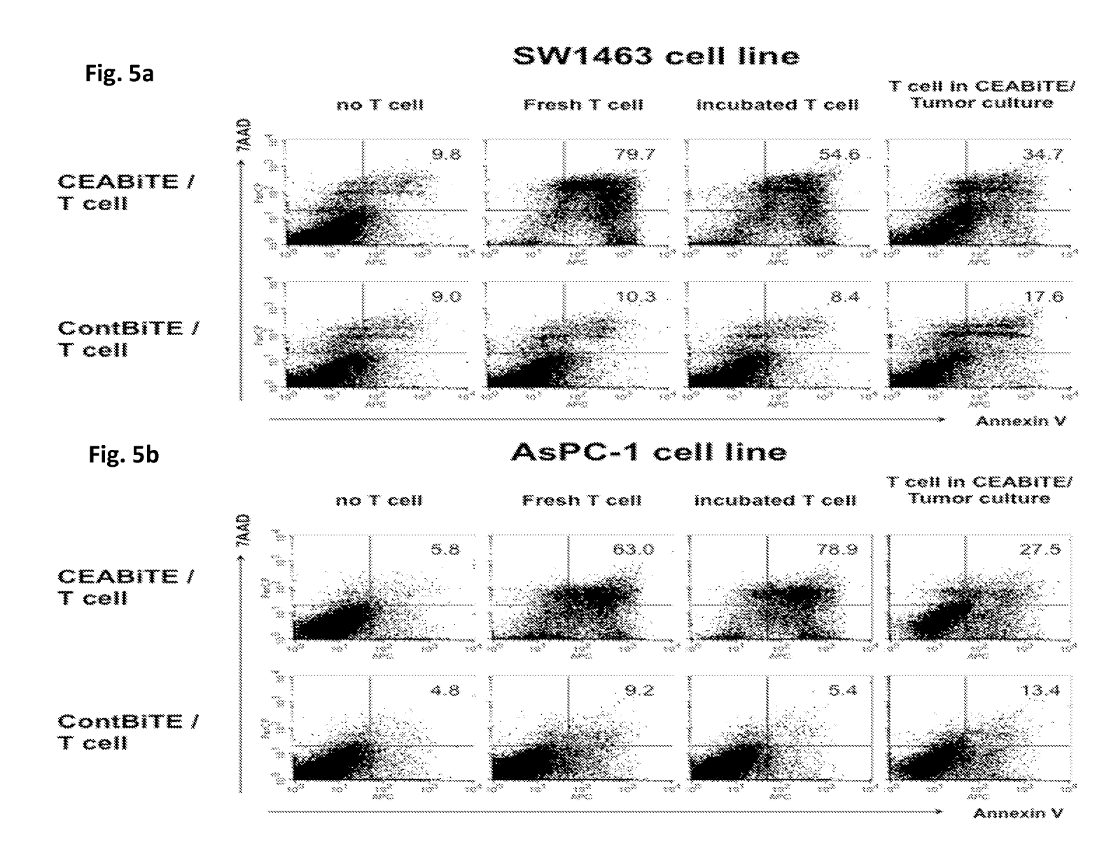

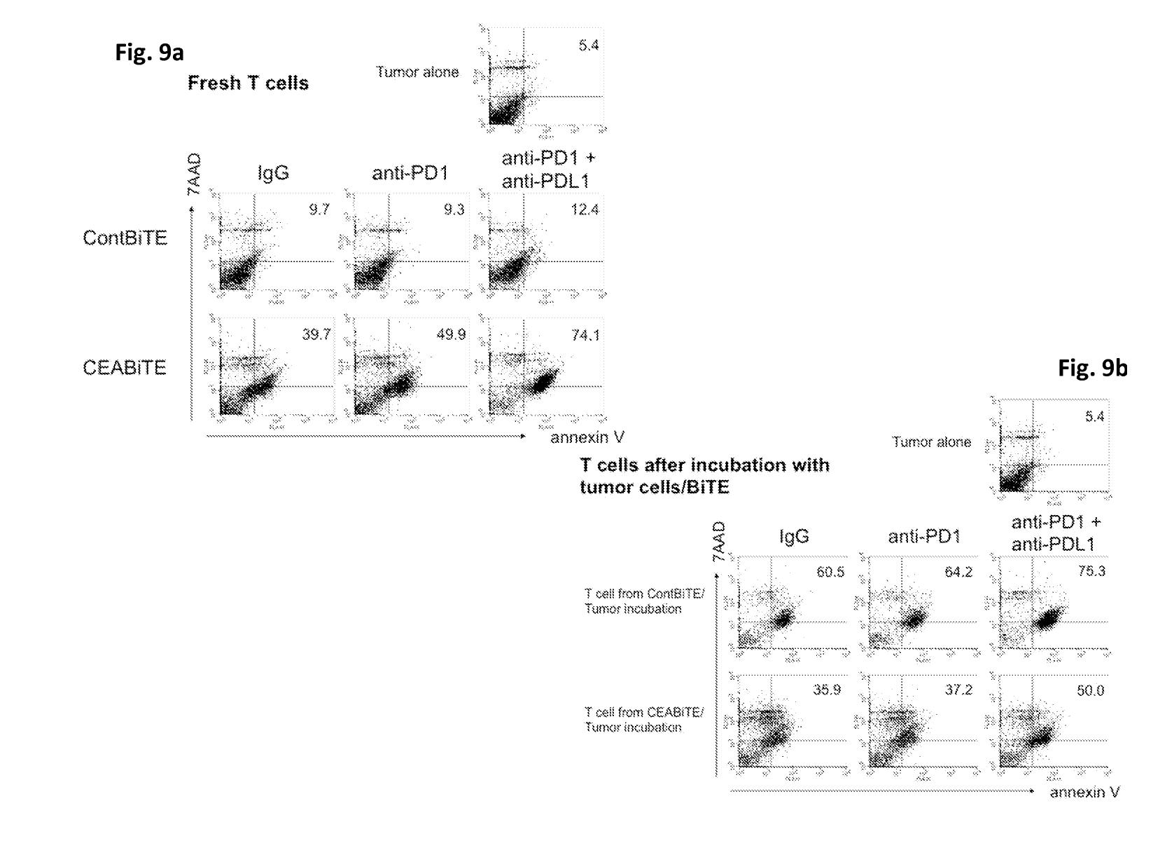

Co-culture of human T cells and tumors in the presence of MEDI-565 CEA-BiTE resulted in 24-44% lysis compared to Cont-BiTE (a control BiTE corresponding to a MEC14 BiTE, that contains a murine anti-Mecoprop (an herbicide) single-chain antibody linked to the same anti-CD3e single-chain antibody as MEDI-565). Another 22-25% of the residual tumor cells were lysed after repeat exposure to fresh T cells plus MEDI-565, suggesting that some tumor cells remained resistant to killing. T cells that had participated in one round of MEDI-565-mediated killing of tumors cells had diminished cytolytic activity when re-exposed to fresh tumor cells in the presence of MEDI-565 (see, e.g., FIG. 5). In addition, by day 1 of the initial co-culture, T cells had upregulated expression of PD1 and tumor cells had increased PD-L1 expression. Repeating these experiments in the presence of anti-PD1 and anti-PD-L1 antibodies, demonstrated that T cell cytolytic activity could be maximized by early blockade of the PD1/PD-L1 signaling. Combined blockade with both anti-PD1 and anti-PD-L1 antibodies further enhanced cytolytic activity of these T cells. Indeed, enhanced T cell responses were maximized when blockade was applied with the initial co-culture (see, e.g., FIG. 4 and FIG. 9).

Early dual blockade of PD1 and PD-L1 partially reverses T cell inhibition induced during CEA-BiTE mediated tumor killing.

### Introduction

The T cell response to colon and pancreatic cancers is associated with improved clinical outcome (Pages et al., New Engl. J. Med., 353:2654-66 (2005) and Nomi et al., Clin. Can. Res., 13:2151-7 (2007)). Methods for directing T cells against these cancers have included both vaccines to activate tumor antigen-specific T cell responses and more complex adoptive immunotherapy strategies with ex vivo expanded T cells. See, e.g., Karlsson et al., Ann. Surg. Oncol., 17:1747-57 (2010); Kobari et al., Brit. J. Surg. 2000; 87:43-8 (2000); and Mosolits et al., Annals of Oncology 2005; 16:847-62 (2005).

Challenges for these therapeutic modalities have included low rates of antigen-specific T cell responses induced by the current generation of tumor vaccines and the complex logistics of in vitro cellular processing and expansion techniques resulting in variable yields of cellular products. See, e.g., Dudley et al., J. Immunother. 26:332 (2003). One potential strategy for overcoming both these challenges is through in vivo recruitment of tumor specific T cells using bispecific single chain-antibodies, such as BiTE antibodies, that simultaneously bind CD3+ T cells via one idiotype, and bind tumors via tumor antigen recognized by the second idiotype. For example, blinatumomab, a bispecific CD19/CD3 antibody, has demonstrated activity in clinical trials for CD19-expressing refractory non-Hodgkin's lymphoma (NHL) and B-cell acute lymphoblastic leukemia (ALL). See, e.g., Bargou et al., Science 2008; 321:974-7 (2008); Klinger et al., Blood, 119:6226-33 (2012).

MEDI-565 (CEA/CD3-bispecific T-cell-engaging BiTE), is composed of a humanized single-chain antibody recognizing carcinoembryonic antigen (CEA, CD66e and CEACAM5), expressed by colorectal, pancreas, and other epithelial malignancies and a de-immunized single-chain antibody specific for human CD3E. In preclinical models, MEDI-565 controlled tumor growth. Lutterbuese et al., J. of Immunotherapy 32:341 (2009). MEDI-565 recognizes and lyses in vitro metastatic colorectal cancer cell explants that had been isolated from patients with chemotherapy-refractory disease. Nonetheless, some tumor cells persistently survived repeated exposure to T cells and MEDI-565-killing in vitro. Osada et al., Brit. J. Can. 102:124-33 (2009). This observation could be due to downregulated T cell responses promulgated by tumors using a number of strategies including antigen escape via tumor antigen down-regulation, secretion of immunoinhibitory cytokines, and activation of regulatory T cells (Tregs) or myeloid-derived suppressor cells (MDSCs). Khong et al., Nat. Immunol. 3:999-1005 (2002); Derynck et al., Nat Genet 29:117-29 (2001); Ferrone et al., Surg. Onc. Clin. of North America 16:755-74 (2007).

### Materials and Methods

### Reagents

PerCP-conjugated anti-CD4 mAb, APC-conjugated anti-CD8 mAb, PE-conjugated anti-CD69, anti-CD25, anti-CD28, anti-CTLA-4, anti-PD1, anti-PD-L1 mAbs, and streptavidin-APC were purchased from BD Biosciences (San Jose, Calif.). FITC-labeled and PE-labeled anti-CEA mAbs from Sanquin (Amsterdam, Netherlands), and 7-AAD and Annexin V-biotin kit were purchased from Immunotech (Marseille, France, cat# PN IM3422). For the analysis of CEACAM5 expression, anti-CEACAM5 mAb (clone 26.5.1, GENOVAC, Germany) was used. Anti-PD1 (clone J116), anti-PD-L1 (clone MIH1), and mouse IgG1κ isotype control (P3.6.2.8.1) were purchased from EBioscience (San Diego, Calif.).

MEDI-565 and the control MEC14 BiTE were constructed as described elsewhere. Osada et al., Brit. J. Can. 102:124-33 (2009). MEDI-565 is composed of a humanized anti-CEA single-chain antibody and a ‘de-immunized’ human CD3e-specific single-chain antibody derived from the mouse monoclonal antibody L2K. See e.g., Chester et al., Intl. J. Can. 57:67-72 (1994). MEC14 BiTE is composed of a murine anti-Mecoprop (a herbicide) single-chain antibody linked to the same anti-CD3e single-chain antibody used to construct MEDI-565. See e.g., Brischwein et al., Molecular immunology 43:1129-43 (2006).

### Tumor Cell Lines

CEA-expressing colon carcinoma cell lines, SW1463 (ATCC CCL-234), Colo205 (ATCC CCL-222), HT29 (ATCC HTB-38), and a CEA-positive pancreatic adenocarcinoma cell line AsPC-1 (ATCC CRL-1682) were purchased from ATCC (Manassas, Va.). The cell lines were cultured in RPMI1640 medium supplemented with 10% fetal bovine serum (Invitrogen Life Technologies, Carlsbad, Calif.). Colorectal cancer cells (CRC057 and CRC096) were isolated from metastatic lesions of cancer patients and propagated in NOD.CB17-Prkdc^{scid}/J (NOD/SCID) mice as described elsewhere. Osada et al., Brit. J. Can. 102:124-33 (2009).

### Flow-Based Cytotoxicity Assay

T cells were negatively isolated from normal donor PBMCs using a T cell isolation kit (Invitrogen Dynal AS, Oslo, Norway, cat#113.11D). In all experiments, purity of CD3 positive cells exceeded 95% of the CD45 positive leukocyte population after isolation procedures. For the cytotoxicity assays, 1×10⁶ tumor cells and 5×10⁶ T cells were added to T75 flasks with MEDI-565 or Cont BiTE (100 ng/mL). After 5 days incubation, all cells were harvested with 0.05% trypsin/EDTA and spun down by centrifugation. Cells were then stained with anti-CD3-FITC or anti-CD45-FITC, 7-AAD, and Annexin V-APC, and CD3 negative or CD45 negative tumor cells were analyzed for expression of Annexin V as a marker of apoptosis using a FACSCalibur™ flow cytometer (BD Biosciences).

### Selection of Tumor Cells Escaping MEDI-565 Mediated T Cell Killing

Tumor cells (5×10⁶ cells) were incubated with negatively isolated T cells (25×10⁶ cells) in T75 flasks in the presence of MEDI-565 (100 ng/mL). After a 5 day incubation (1st round), floating cells were discarded and tumor cells adherent to the flask were harvested by trypsin/EDTA treatment. Cytotoxicity was analyzed by flow cytometry based on Annexin V/7-AAD staining as described above. Tumor cells were centrifuged with Ficoll-Paque and viable tumor cells were isolated and transferred into the flask (5×10⁶ cells/T-75 flask) together with T cells isolated from the same donors' PBMCs of the 1st round co-incubation. After this 2nd round co-incubation, tumor cell viability was analyzed by flow cytometry again. To monitor CEA expression level of tumor cells, cells were labeled with anti-CEACAM5 mAb (clone 26.5.3), incubated for 30 min at 4° C., then washed and stained with PE-labeled goat anti-mouse IgG antibody for 30 min.

### Regulatory T Cell Analysis in MEDI-565 Augmented T Cell-Mediated Killing

Tumor cells (SW1463, AsPC-1) were co-incubated with T cells at a 5:1 ratio in the presence of MEDI-565 or Cont-BiTE (100 ng/mL). After 5 days of incubation, cells were harvested from the plates, fixed, and permeabilized. Cells were stained with anti-CD25-FITC/anti-CD4-PerCP/anti-CD3-APC and PE-labeled anti-Foxp3 or control IgG using a Foxp3 staining kit (eBiosciences). Percentages of CD25+ and Foxp3+ cells within the CD3+CD4+ T cell population were analyzed.

Cytotoxic Function of T Cells after Longer-Term Co-Incubation with Tumor Cells in the Presence of MEDI-565

Negatively isolated T cells from normal donor PBMCs were incubated with tumor cells (SW1463, AsPC-1) at a 5:1 ratio in the presence of MEDI-565 or Cont-BiTE (100 ng/mL) for 5 days. Floating cells were collected, and density gradient centrifugation with Ficoll-Paque was performed to obtain viable T cells only. As controls, fresh T cells isolated from the same normal donors, and in some experiments, 5-day incubated T cells without tumor cells/BiTE antibody, were used. Soon after isolation of viable T cells, co-incubation of these T cells and tumor cells at a 5:1 ratio was initiated in the presence of MEDI-565 or Cont-BiTE (100 ng/mL). After a 5 day incubation, tumor cells were harvested and the percentage of Annexin V-positive tumor cells was measured by a flow-based cytotoxicity assay.

### Expression of Co-Stimulatory Molecules on T Cells and Tumor Cells Following MEDI-565-T Cell-Mediated Killing

SW1463 cells were co-incubated with T cells derived from a normal donor at a 1:5 ratio, in the presence of MEDI-565 or Cont-BiTE (100 ng/mL) for 1, 3, 5, or 7 days. Controls consisted of T cells or tumor cells incubated alone in the presence of MEDI-565 or Cont-BiTE. Cells were harvested with trypsin/EDTA, and stained with anti-CD45-APC, and PE-conjugated antibodies for CD28, CTLA-4, PD1, or PD-L1. PE-conjugated mouse IgG was used as negative control staining. Cells were acquired via FACSCalibur and analyzed with CellQuest software. Tumor cells were identified as CD45⁻ and T cells as CD45⁺

Restoration of Cytolytic T Cell Activity with Anti-PD1 and Anti-PD-L1

T cells isolated from PBMCs of a normal donor were incubated with SW1463 cells in the presence of MEDI-565 (a CEA-BITE) or Cont-BiTE (100 ng/mL) for 7 days after which the viable T cells were isolated by density gradient centrifugation with Ficoll-Paque, and used as effector cells for a second round of co-incubation with SW1463 cells in the presence of either MEDI-565 or Cont-BiTE. To assess the role of PD1 and PD-L1 in T cell exhaustion, anti-PD1 blocking mAb, anti-PD-L1 blocking mAb, or a combination of both antibodies were added to the culture (final concentration: 5 μg/mL). Controls consisted of isotypic IgG (final concentration: 5 μg/mL) added to parallel cultures. After 5 days incubation, the flow-based cytotoxicity assay was performed as described above.

### Results

A subset of CEA-expressing tumors resist killing despite repeated exposure to T cells and MEDI-565 in vitro

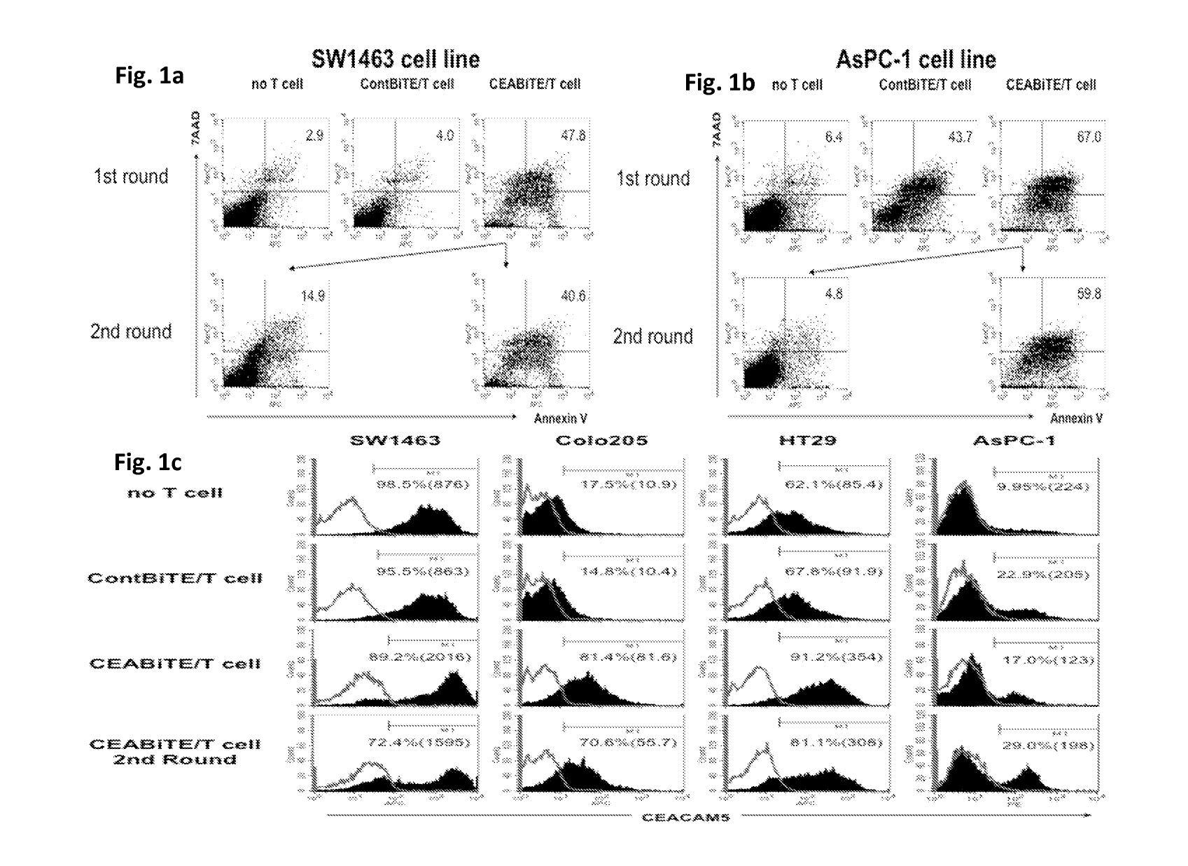

We first confirmed CEA-expressing tumor cells survived despite repeated exposure to T cells and MEDI-565 in vitro. To control for T cell function, we tested the viability of the CEA-expressing colorectal cell line SW1463 following 2 cycles of co-culture with normal donor PBMCs and MEDI-565. In a representative experiment, we observed that cytotoxicity was 47.8% (vs. 2.9% for tumor alone) after one round of attack. Viable tumor cells were isolated and re-exposed to a second aliquot of the same donor PBMCs and MEDI-565. After this second round of co-culture with fresh T cells and MEDI-565, 40.6% (compared with 14.9% in the absence of MEDI-565) of the SW1463 tumor cells were killed (FIG. 1A). A similar trend was seen with AsPC-1 (FIG. 1B). Tumor cells that survived the first round of MEDI-565-mediated T cell killing showed similar levels of survival following the second round of T-cell/MEDI-565 exposure. These data suggest that tumor cells not killed in the first exposure remained partially susceptible to immune attack. Additional cycles of exposure to normal donor PBMC and MEDI-565 did not produce additional killing, suggesting that a subset of tumor cells were resistant to cytolysis

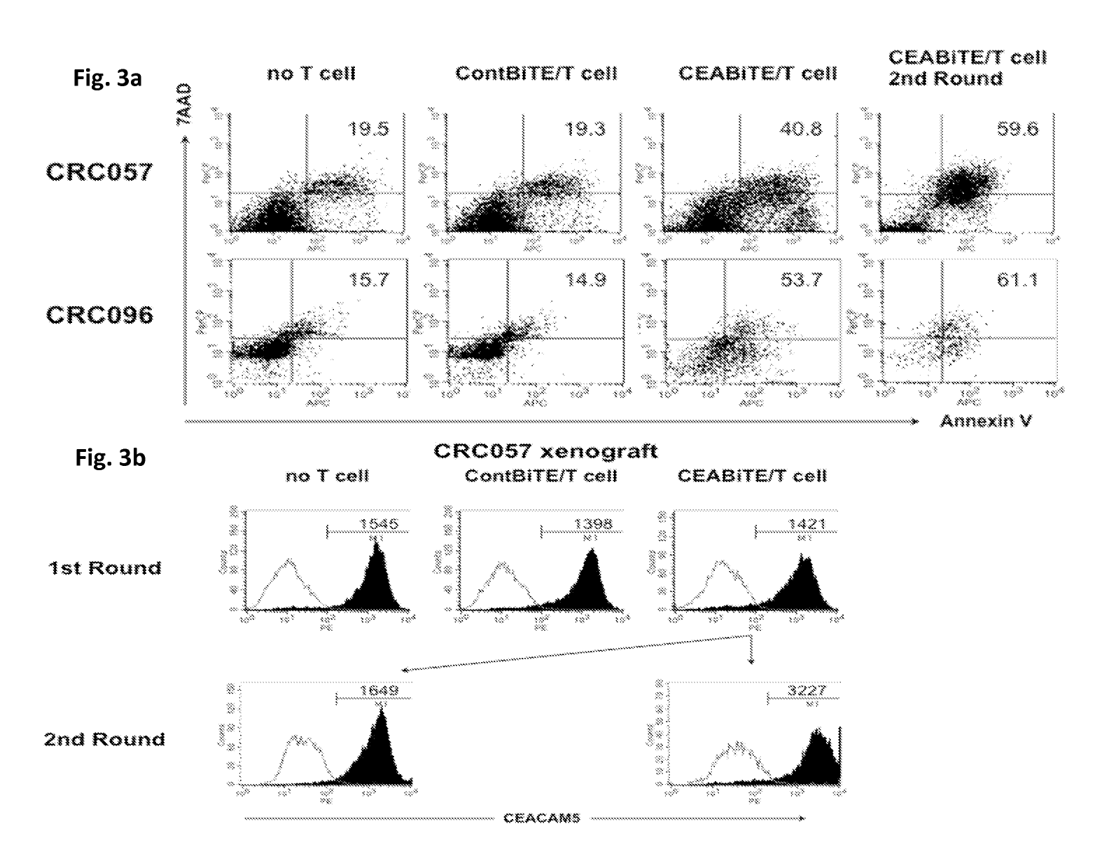

To determine if the mechanism of tumor cell survival was due to downregulation of the target antigen, tumor-specific CEA expression measured by mean fluorescence intensity (MFI), was shown to have increased at the time of the second co-culture. This increase in CEA expression level was not observed with incubation of tumor cells alone or with co-incubation of T cells and tumor cells without MEDI-565 CEA-BITE (FIG. 1C). Furthermore, this effect was not unique to established colorectal cancer cell lines and was observed in primary culture of human colorectal cancer cells as well. In explants CRC057 and CRC096, the MFI for CEA increased by 109% (1545 to 3227 MFI) and 48% (2500 to 3700 MFI), respectively, after the second incubation of MEDI-565+ T cells (FIG. 3). Despite an increase in CEA expression, a substantial fraction of the cells remained alive after repeat exposure to T cells/MEDI-565, suggesting partial resistance of tumor to MEDI-565-mediated T cell killing.

Diminished T-Cell Activity Over Time after Repeated MEDI-565 Exposure

Another potential mechanism for tumor cell escape from CEA-BITE-mediated T cell killing could be T cell exhaustion. See, e.g., Baeuerle et al., Can. Res. 69:4941-4 (2009). To determine the viability and function of T cells previously co-cultured with MEDI-565 and tumor cell lines SW1463 and AsPC-1, viable T cells were harvested and used as effector cells in repeat incubations with fresh tumor cells to assess for possible T-cell exhaustion with repeated CEA-BITE-mediated killing. Using matched PBMC for the same donor, fresh T cells induced 79.7% killing after a 5 day co-incubation with SW1463 cells in the presence of MEDI-565, whereas only 34.7% killing was noted using T cells that had previously participated in MEDI-565-mediated attack. With AsPC-1 cells under similar conditions, fresh T cells induced 63.0% killing vs. 27.5% in T cells that had previously been co-cultured with MEDI-565 (FIG. 5). Thus, viable T cells previously exposed to tumor cells showed diminished killing activity compared to fresh T cells, suggesting immunoregulatory mechanisms may be reducing the cytotoxic efficacy of T cells

### Effect of MEDI-565 on Regulatory T Cell

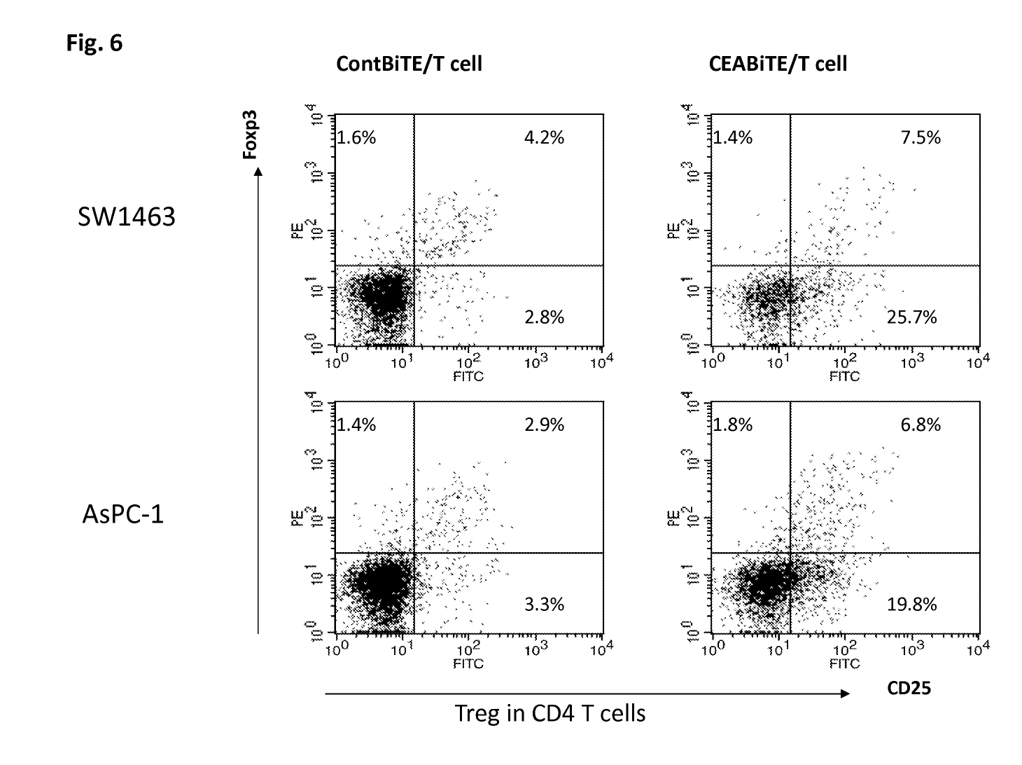

One mechanism for tumor-associated immunomodulation of T cell function is through expansion of regulatory T cells which are frequently increased in the tumor milieu. See, e.g., Facciabene et al., Can. Res. 72:2162-71 (2012). To investigate the potential for CEA-BITE to expand regulatory T cells in vitro, Treg populations were enumerated by flow cytometry in co-culture experiments with and without MEDI-565. Specifically, the frequency of both CD4+CD25+FoxP3-effector T cells and CD4+CD25+FoxP3+Tregs were analyzed after a 5 day coincubation of tumor cells, T cells and either CEA-BITE or Cont-BiTE. An increase in activated CD4+CD25+FoxP3-effector T cells was observed (2.8% vs. 25.7% in SW1463 cultures, 3.3% vs. 19.8% in AsPC-1 cultures). In addition, MEDI-565-mediated T cell killing of tumor cells induced significant increases in Treg levels (4.2% vs. 7.5% in SW1463 cultures, 2.9% vs. 6.8% in AsPC-1 cultures) (FIG. 6). Because of the potent immunosuppressive role of Tregs, this suggests that tumor escape from CEA-BITE-mediated T cell killing might be caused, at least in part, by the increased Treg population.

### Enhanced PD1 and PD-L1 Expression in MEDI-565-Mediated T Cell Killing of Tumor Cells

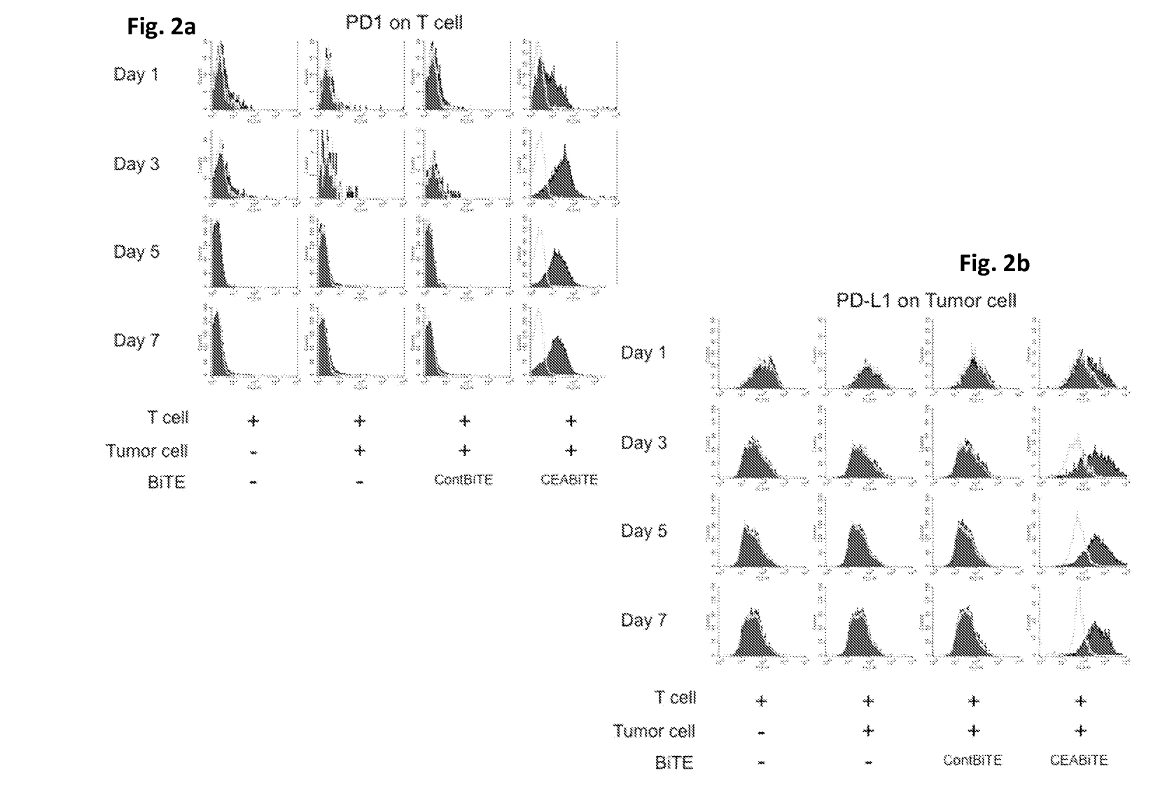

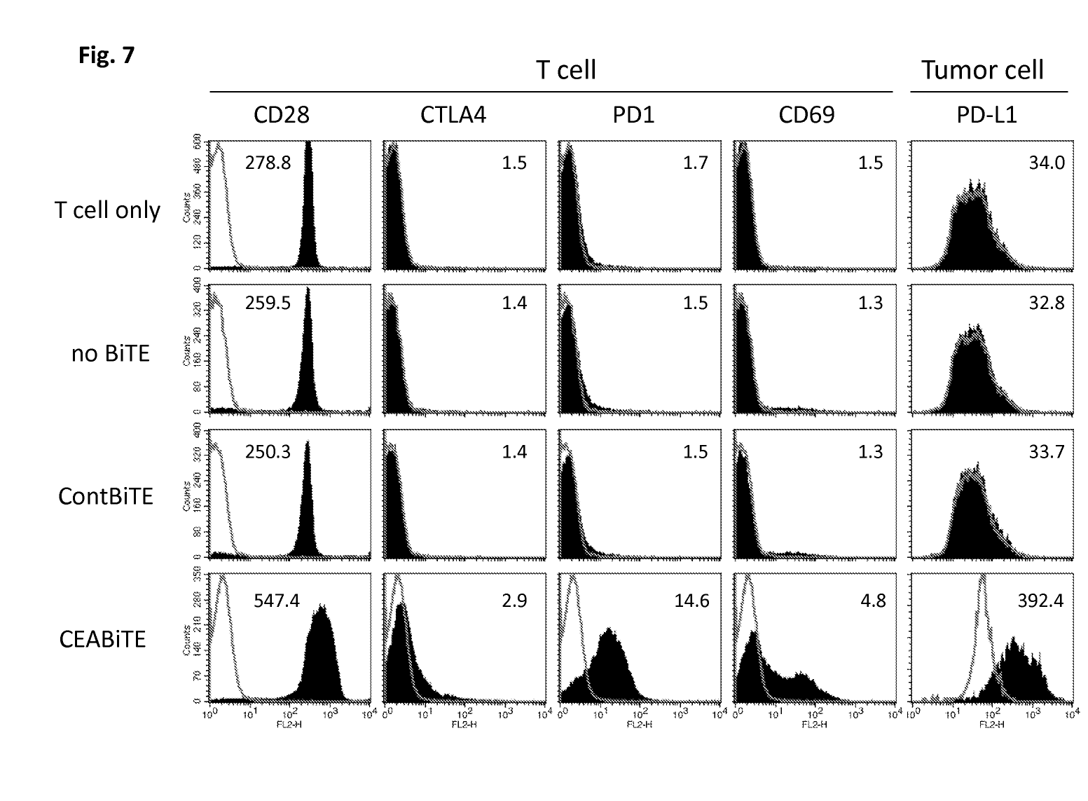

In addition to increased Tregs, another possible mechanism for T cell functional decline following co-culture with tumor and MEDI-565 is expression of PD1 by T cells and PD-L1 by tumor cells leading to T cell exhaustion. T cells were assayed for CTLA4 and PD1 expression after 7 days of MEDI-565-mediated T cell attack of SW1463 (FIG. 7). We found that PD1/PD-L1 upregulation is an early event that occurs in the combined cultures within days of the first cycle of MEDI-565-mediated T cell attack. The change of PD1 expression on T cells over time is shown in FIG. 2A. While CTLA4 expression was minimally changed, PD1 expression significantly increased as well as CD28 (a receptor for both B7.1 and B7.2—members of the CTLA4 immune checkpoint pathway) and CD69 a marker of T-cell activation.

In addition to the increase in PD1 expression in T cells, tumor cells increased PD-L1 expression during the 7 day period (FIG. 7 and FIG. 2B). These changes in PD1 and PD-L1 expression did not occur following co-incubation of T cells and tumor cells in the absence of MEDI-565, suggesting that this may be a potential mechanism of immune modulation, and a possible opportunity to reverse this dysfunction. These data suggest that secreted factors from tumor cells, such as cytokines, may not be the direct cause of T cell exhaustion, but rather, that up-regulation of PD1 on T cells and PD-L1 on tumor cells contribute to T-cell exhaustion and decreased tumoricidal activity. Furthermore, PD1/PD-L1 upregulation is an early event that occurs within a day of the first cycle of MEDI-565-mediated T cell attack.

### Effect of Anti-PD1 and Anti-PD-L1 on T-Cell Activity

In order to determine the importance of PD1 and PD-L1 in T cell exhaustion and whether exhaustion could be abrogated, we tested inhibition of the PD1/PD-L1 immune checkpoint pathway in tumor cells exposed to T cells and MEDI-565 using blocking antibodies specific to either PD1 or PD-L1. Early blocking of PD1 on fresh T cells led to increased MEDI-565-mediated cytolysis of SW1463 (66.3%) relative to fresh T cells exposed to MEDI-565 only (30.7%).

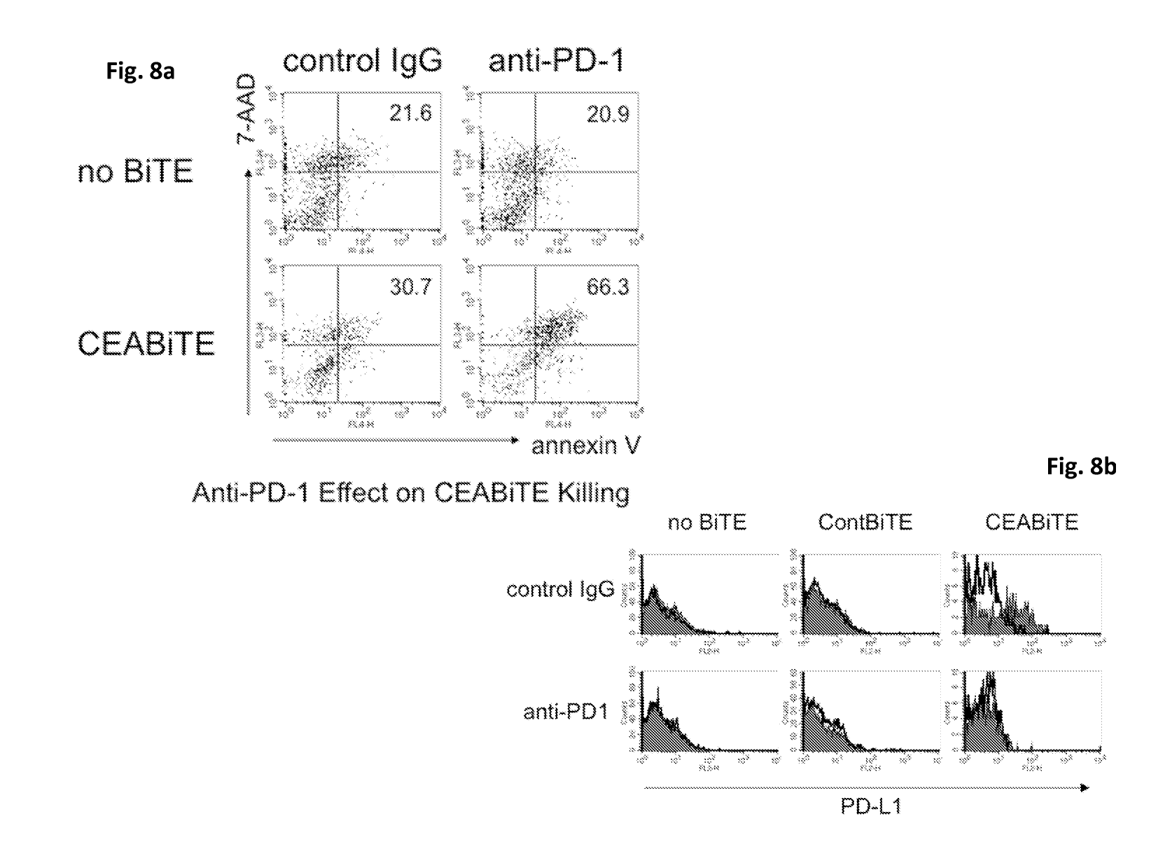

As noted previously, when SW1463 cells were cultured with MEDI-565 for one round of T-cell mediated attack, PD-L1 expression increased on tumor cells (FIG. 8). However, when anti-PD1 antibody was added to the culture, this increase in PD-L1 expression on tumor was inhibited.

These T-cells were then isolated and recultured for a second round of MEDI-565 mediated attack in combination with IgG control, anti-PD1, anti-PD-L1, or dual anti-PD1/anti-PD-L1. As expected, in the absence of inhibition of either PD1 or PD-L1, T-cell cytolysis was reduced from 39.7% to 35.9% after one round of MEDI-565 attack. In contrast, inhibition of PD1 or PD-L1 alone on fresh T cells led to persistently high tumor-specific cytolytic activity of 49.9%, and the dual PD1/PD-L1 inhibition led to maximum level of tumoricidal activity of 74.1%. This high level of tumoricidal activity was also seen in MEDI-565-naive T cells (75.3%), which were incubated for the same time period with tumor cells in the presence of Cont-BiTE.

Once PD1 upregulation had already occurred on T cells, cytolytic activity was diminished despite intervention with anti-PD1. Specifically, T-cells previously incubated with MEDI-565 and tumor in the absence of PD1/PD-L1 blockade and then reincubated for a second round of tumor cytolysis, had decreased cytolytic efficacy even when PD1/PD-L1 blockage was instituted. For example, in the presence of anti-PD1 (37.1% vs. 49.9% for fresh T cells exposed to MEDI-565) or anti-PD1/anti-PD-L1 (50% vs. 74.1% for fresh T cells exposed to MEDI-565) (FIG. 9 and FIG. 4) tumor killing was persistently inferior to killing with fresh T cells. These data suggest that the effects of PD1 and PD-L1 on T cell function may be partly inhibited by immune checkpoint blockade, but immunomodulation cannot easily be reversed in this in vitro assay suggesting that early or prophylactic intervention is required to maximize T-cell mediated cytolysis of tumor.

### Discussion

We demonstrate in an in vitro model where all effector T cells have the potential to mediate anti-tumor activity that the tumor may remain partially resistant to multiple rounds of T cell-mediated killing. A commonly reported mechanism for tumor escape, loss of the target antigen (CEA), did not occur. In contrast, we did observe increased expression of CEA, an increase the percentage of Tregs and up-regulation of the PD1/PD-L1 immune checkpoint pathway. Blockade of this immune checkpoint, particularly early exposure to the anti-PD1 and anti-PD-L1 mAbs, was the most effective strategy for restoring T cell function.

The role of the immune system, in particular T cell-mediated cytotoxicity, in tumor control is well recognized. Immune checkpoints (e.g., CTLA-4, PD1, and PD-L1) normally act on T-cells to temper the immune response as a means to control autoimmunity, but are coopted by tumors to escape immune surveillance. The PD1/PD-L1 pathway has been implicated as one of many potential immunoregulatory pathways important in T-cell “exhaustion” facilitating an immunosuppressive environment for tumor growth and progression. See, e.g., Skauishi et al., J Exp. Med. 207:2187-94 (2010). PD1, expressed on CD4+ T cells, CD8+ T cells, NK-T cells, B cells, and activated monocytes, binds to its ligand PD-L1, expressed on tumor cells, somatic cells especially in immune privileged sites (eye, ovary, placenta) and immune cells such as macrophages and myeloid-derived suppressor cells, to downregulate T cell activity. See, e.g., Sharpe et al., Nat, Immunol. 8:239-45 (2007). Solid tumors such as melanoma, renal cell carcinoma, and non-small cell lung cancers with elevated PD-L1 expression have shown impressive clinical responses to therapies that disrupt the PD1/PD-L1 immune checkpoint pathway. See, e.g., Ribas A., New Engl. J. Med., 367(12):1168 (2012).

Interferon-gamma plays an important role in up-regulation of PD1 and PD-L1 expression, and we previously reported the robust increase of interferon-gamma in co-cultures of tumor, T cells, and MEDI-565. See, e.g., Curran et al., PNAS 107:4275-80 (2010). PD-L1 up-regulation to interferon-gamma protects native cells from autoimmune attack during an immune response against infection or malignancy, but the ability of some tumors to coopt this mechanism impairs the ability of the T cell infiltrate to eradicate tumor. See, Id. Tumor up-regulation of PD-L1 in the presence of cytotoxic T cells is a reported mechanism of tumor immunologic escape. See, e.g., Iwai et al., PNAS 99:12293-7 (2002) and Weber et al., The Oncol. 13:16-25 (2008). Inhibition of this immune checkpoint with anti-PD1/anti-PD-L1 antibody therapy restored T-cell cytolysis in our experiments. Our data suggests that up-regulation of the PD1/PD-L1 immune checkpoint pathway is an early event that can occur within one day of T-cell exposure to tumor, and that early intervention with therapeutic immune checkpoint inhibition can abrogate tumor resistance. As anti-PD1 and anti-PD-L1 dual blockade can only partially restore T cell cytolysis against tumor, other immune checkpoints or mechanisms of resistance to T cell killing may be concomitantly up-regulated with PD1.

One important aspect of our study is the use of BiTE antibodies to arm all the T cells in culture against the target antigen. This permits analysis of influences on T cell cytolytic activity without the potential confounding effects of poor T cell stimulation or trafficking to tumor. MEDI-565 is currently being studied in a phase I clinical trial and immune correlates of protection are being assessed. Blinatumomab, a CD19 targeting BiTE being studied in refractory hematologic malignancies, has shown promising activity with durable molecular remissions lasting 3 years in B-cell ALL. See, e.g., Topp et al., Blood 120:5185-7 (2012) To date, mechanisms of resistance to this BiTE antibody, as well as profiling of PD1/PD-L1 expression on patients treated with blinatumomab, have not been reported. Finally, based on the demonstration of clinical activity of single agent T cell immune checkpoint inhibitors targeting CTLA4 (ipilimumab), PD1, and PD-L1, clinical trials of combination therapies with other immune therapies are ongoing. See, e.g., Hodi et al., New Engl. J. Med. 363:711-723 (2010), Tapolian et al., New Engl. J. Med. 366:2443-2454 (2012), and Brahmer et al., New Engl. J. Med. 366:2455-2465 (2012).

Those skilled in the art will recognize, or be able to ascertain using no more than routine experimentation, many equivalents to the specific aspects of the disclosure described herein. Such equivalents are intended to be encompassed by the following claims.

Various publications are cited herein, the disclosures of which are incorporated by reference in their entireties.

Although the foregoing invention has been described in some detail by way of illustration and example for purposes of clarity of understanding, it will be obvious that certain changes and modifications can be practiced within the scope of the appended claims.

1. A method of killing target cells in a cell population, comprising contacting a cell population containing target cells expressing a target associated antigen and T cells with (a) 1, 2, or more immune checkpoint antagonists (ImCpAnts) that specifically bind 2 or more different targets of an immune checkpoint pathway and (b) a multispecific T cell-redirecting agent (MsTC-Redir) that (i) specifically binds the target associated antigen expressed on the target cells and (ii) specifically binds a T cell surface antigen, wherein the contacting of the cell population with (a) and (b) leads to death of target cells.

2-52. (canceled)

53. The method of claim 1, wherein the target cells are tumor cells, immune cells, or an infectious agent.

54. The method of claim 53, wherein the tumor cells are from an epithelial tumor.

55. The method of claim 54, wherein the tumor cells are from a leukemia, lymphoma, melanoma, renal cell carcinoma, non-small cell lung cancer, colon cancer, pancreatic cancer, esophageal cancer, gastric cancer or a colorectal cancer.

56. The method of claim 55, wherein the tumor cells are CEA (CEACAM5) expressing tumor cells.

57. The method of claim 1, wherein the cell population is contacted with 1, 2 or more ImCpAnts before the cell population is contacted with the MsTC-Redir.

58. The method of claim 1, wherein the cell population is contacted with 1, 2 or more ImCpAnts at about ½, 1, 2, 3, 4, 5, 6, 7, 8, 9, 10, 11, 12, 18, 24, 36, 48, 60 or 96 hours before the cell population is contacted with the MsTC-Redir.

59. The method of claim 1, wherein the cell population is contacted with 1, 2 or more ImCpAnts at about ½ hour to about 3 weeks, about ½ hour to about 2 weeks or about ½ hour to about 1 week before the cell population is contacted with the MsTC-Redir.

60. The method of claim 1, wherein the cell population is contacted with 1, 2 or more ImCpAnts at about the same time as the cell population is contacted with the MsTC-Redir.

61. The method of claim 1, wherein the cell population is contacted with 1, 2 or more ImCpAnts within 6 hours of the cell population being contacted with the MsTC-Redir.

62. The method of claim 1, wherein the ImCpAnts include at least 1, 2 or more of: an anti-PD1 antibody or antigen binding fragment thereof, an anti-PD-L1 antibody or antigen binding fragment thereof, an anti-PD-L2 antibody or antigen binding fragment thereof, an anti-CTLA4 antibody or antigen binding fragment thereof, an anti-B7.1 antibody or antigen binding fragment thereof, an anti-B7.2 antibody or antigen binding fragment thereof, and an anti-B7H2 antibody or antigen binding fragment thereof.

63. The method of claim 1, wherein the ImCpAnts include 1, 2 or more antibodies or antigen binding fragments thereof, that specifically bind one, two, three or more targets selected from BTLA, PDH1, B7H3, B7H4, TIM3, A2aR, and LAG3.

64. The method of claim 1, wherein the MsTC-Redir binds a CD3/TCR complex expressed on the surface of a T cell.

65. The method of claim 1, wherein the ImCpAnts and/or MsTC-Redir is a bispecific antibody.

66. The method of claim 65, wherein the bispecific antibody is a member selected from the group consisting of a bispecific diabody, a single-chain bispecific diabody, a single chain bispecific tandem variable domain, a bispecific single domain antibody, a bispecific F(ab′)2, a dock-and-lock bivalent or trivalent Fab, a bispecific (mab)₁, and a bispecific (mab)₂.

67. The method of claim 66, wherein the bispecific antibody is a bi-specific T-cell engager (BiTE).

68. The method of claim 67, wherein the BiTE competes with an antibody or antigen binding fragment thereof comprising the amino acid sequence of SEQ ID NO:3.

69. The method of claim 68, wherein the BiTE is an antibody or antigen binding fragment thereof comprising the amino acid sequence SEQ ID NO:3.

70. The method of claim 1, wherein the cell population is contacted with the ImCpAnts in vitro, ex vivo, or in vivo.

71. The method of claim 1, wherein the cell population is contacted with the MsTC-Redir in vitro, ex vivo, or in vivo.

72. A method of enhancing antitumor immunity in a subject comprising co-administering to a subject a bi-specific T-cell engager (BiTE) and two or more ImCpAnts.

73. A method of reducing resistance of a tumor cell to T cell mediated killing in a subject comprising co-administering to the subject a bi-specific T-cell engager (BiTE) and two or more ImCpAnts.

74. The method of claim 73, wherein the BiTE competes with an antibody or antigen binding fragment thereof comprising the amino acid sequence of SEQ ID NO:3.

75. The method of claim 73, wherein the BiTE is an antibody or antigen binding fragment thereof comprising the amino acid sequence SEQ ID NO:3.

76. The method of claim 73, wherein the subject is administered 1, 2, or more ImCpAnts at about ½ hour to about 3 weeks, about ½ hour to about 2 weeks or about ½ hour to about 1 week before the subject is administered the BiTE.

77. A method of enhancing antitumor immunity in a subject comprising co-administering to a subject a bi-specific T-cell engager (BiTE) and one, two or more ImActAgs.

78. The method of claim 77, wherein the BiTE competes with an antibody or antigen binding fragment thereof comprising the amino acid sequence of SEQ ID NO:3.

79. The method of claim 77, wherein the BiTE is an antibody or antigen binding fragment thereof comprising the amino acid sequence SEQ ID NO:3.

80. The method of claim 77, wherein the subject is administered 1, 2, or more ImCpAnts at about ½ hour to about 3 weeks, about ½ hour to about 2 weeks or about ½ hour to about 1 week before the subject is administered the BiTE.

81. A method of modulating and redirecting an immune response to a diseased cell or tissue and/or an immune cell in a subject, comprising, administering to the subject (a) 1, 2, or more immune checkpoint antagonists (ImCpAnts) that specifically bind 2 or more different targets of an immune checkpoint pathway and (b) a multispecific T cell-redirecting agent (MsTC-Redir) that (i) specifically binds an antigen on the surface of the diseased cell or tissue and/or an immune cell and (ii) specifically binds a T cell surface antigen.


SEQUENCE LISTING (121 sequences)

SEQ ID NO: 1 - protein, 10 aa, Artificial Sequence
  Synthetic peptide
DRGLRFYFDY

SEQ ID NO: 2 - protein, 19 aa, Artificial Sequence
  Synthetic peptide
FILNKANGGTTEYAASVKG

SEQ ID NO: 3 - protein, 501 aa, Artificial Sequence
  Synthetic A240 VL bispecific single chain antibody polypeptide
QAVLTQPASLSASPGASASLTCTLRRGINVGAYSIYWYQQKPGSPPQYLLRYKSDSDKQQGSGVSSRFSASKDASANAGILLISGLQSEDEADYYCMIWHSGASAVFGGGTKLTVLGGGGSGGGGSGGGGSEVQLVESGGGLVQPGRSLRLSCAASGFTVSSYWMHWVRQAPGKGLEWVGFIRNKANGGTTEYAASVKGRFTISRDDSKNTLYLQMNSLRAEDTAVYYCARDRGLRFYFDYWGQGTTVTVSSSGGGGSDVQLVQSGAEVKKPGASVKVSCKASGYTFTRYTMHWVRQAPGQGLEWIGYINPSRGYTNYADSVKGRFTITTDKSTSTAYMELSSLRSEDTATYYCARYYDDHYCLDYWGQGTTVTVSSGEGTSTGSGGSGGSGGADDIVLTQSPATLSLSPGERATLSCRASQSVSYMNWYQQKPGKAPKRWIYDTSKVASGVPARFSGSGSGTDYSLTINSLEAEDAATYYCQQWSSNPLTFGGGTKVEIK

SEQ ID NO: 4 - protein, 702 aa, Homo sapiens
MESPSAPPHRWCIPWQRLLLTASLLTFWNPPTTAKLTIESTPFNVAEGKEVLLLVHNLPQHLFGYSWYKGERVDGNRQIIGYVIGTQQATPGPAYSGREIIYPNASLLIQNIIQNDTGFYTLHVIKSDLVNEEATGQFRVYPELPKPSISSNNSKPVEDKDAVAFTCEPETQDATYLWWVNNQSLPVSPRLQLSNGNRTLTLFNVTRNDTASYKCETQNPVSARRSDSVILNVLYGPDAPTISPLNTSYRSGENLNLSCHAASNPPAQYSWFVNGTFQQSTQELFIPNITVNNSGSYTCQAHNSDTGLNRTTVTTITVYAEPPKPFITSNNSNPVEDEDAVALTCEPEIQNTTYLWWVNNQSLPVSPRLQLSNDNRTLTLLSVTRNDVGPYECGIQNKLSVDHSDPVILNVLYGPDDPTISPSYTYYRPGVNLSLSCHAASNPPAQYSWLIDGNIQQHTQELFISNITEKNSGLYTCQANNSASGHSRTTVKTITVSAELPKPSISSNNSKPVEDKDAVAFTCEPEAQNTTYLWWVNGQSLPVSPRLQLSNGNRTLTLFNVTRNDARAYVCGIQNSVSANRSDPVTLDVLYGPDTPIISPPDSSYLSGANLNLSCHSASNPSPQYSWRINGIPQQHTQVLFIAKITPNNNGTYACFVSNLATGRNNSIVKSITVSASGTSPGLSAGATVGIMIGVLVGVALI

SEQ ID NO: 5 - protein, 524 aa, Homo sapiens
MESPSAPPHRWCIPWQRLLLTASLLTFWNPPTTAKLTIESTPFNVAEGKEVLLLVHNLPQHLFGYSWYKGERVDGNRQIIGYVIGTQQATPGPAYSGREIIYPNASLLIQNIIQNDTGFYTLHVIKSDLVNEEATGQFRVYPELPKPSISSNNSKPVEDKDAVAFTCEPETQDATYLWWVNNQSLPVSPRLQLSNGNRTLTLFNVTRNDTASYKCETQNPVSARRSDSVILNVLYGPDAPTISPLNTSYRSGENLNLSCHAASNPPAQYSWFVNGTFQQSTQELFIPNITVNNSGSYTCQAHNSDTGLNRTTVTTITVYAEPPKPFITSNNSNPVEDEDAVALTCEPEIQNTTYLWWVNNQSLPVSPRLQLSNDNRTLTLLSVTRNDVGPYECGIQNELSVDHSDPVILNVLYGPDDPTISPSYTYYRPGVNLSLSCHAASNPPAQYSWLIDGNIQQHTQELFISNITEKNSGLYTCQANNSASGHSRTTVKTITVSASGTSPGLSAGATVGIMIGVLVGVALI

SEQ ID NO: 6 - protein, 524 aa, Homo sapiens
MESPSAPPHRWCIPWQRLLLTASLLTFWNPPTTAKLTIESTPFNVAEGKEVLLLVHNLPQHLFGYSWYKGERVDGNRQIIGYVIGTQQATPGPAYSGREIIYPNASLLIQNIIQNDTGFYTLHVIKSDLVNEEATGQFRVYPELPKPSISSNNSKPVEDKDAVAFTCEPETQDATYLWWVNNQSLPVSPRLQLSNGNRTLTLFNVTRNDTASYKCETQNPVSARRSDSVILNVLYGPDDPTISPSYTYYRPGVNLSLSCHAASNPPAQYSWLIDGNIQQHTQELFISNITEKNSGLYTCQANNSASGHSRTTVKTITVSAELPKPSISSNNSKPVEDKDAVAFTCEPEAQNTTYLWWVNGQSLPVSPRLQLSNGNRTLTLFNVTRNDARAYVCGIQNSVSANRSDPVTLDVLYGPDTPIISPPDSSYLSGANLNLSCHSASNPSPQYSWRINGIPQQHTQVLFIAKITPNNNGTYACFVSNLATGRNNSIVKSITVSASGTSPGLSAGATVGIMIGVLVGVALI

SEQ ID NO: 7 - protein, 420 aa, Homo sapiens
MESPSAPPHRWCIPWQRLLLTASLLTFWNPPTTAKLTIESTPFNVAEGKEVLLLVHNLPQHLFGYSWYKGERVDGNRQIIGYVIGTQQATPGPAYSGREIIYPNASLLIQNIIQNELSVDHSDPVILNVLYGPDDPTISPSYTYYRPGVNLSLSCHAASNPPAQYSWLIDGNIQQHTQELFISNITEKNSGLYTCQANNSASGHSRTTVKTITVSAELPKPSISSNNSKPVEDKDAVAFTCEPEAQNTTYLWWVNGQSLPVSPRLQLSNGNRTLTLFNVTRNDARAYVCGIQNSVSANRSDPVTLDVLYGPDTPIISPPDSSYLSGANLNLSCHSASNPSPQYSWRINGIPQQHTQVLFIAKITPNNNGTYACFVSNLATGRNNSIVKSITVSASGTSPGLSAGATVGIMIGVLVGVALI

SEQ ID NO: 8 - protein, 108 aa, Homo sapiens
EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIK

SEQ ID NO: 9 - protein, 121 aa, Homo sapiens
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSS

SEQ ID NO: 10 - protein, 5 aa, Homo sapiens
RYWMS

SEQ ID NO: 11 - protein, 17 aa, Homo sapiens
NIKQDGSEKYYVDSVKG

SEQ ID NO: 12 - protein, 12 aa, Homo sapiens
EGGWFGELAFDY

SEQ ID NO: 13 - protein, 12 aa, Homo sapiens
RASQRVSSSYLA

SEQ ID NO: 14 - protein, 7 aa, Homo sapiens
DASSRAT

SEQ ID NO: 15 - protein, 9 aa, Homo sapiens
QQYGSLPWT

SEQ ID NO: 16 - protein, 500 aa, Artificial Sequence
  Bispecific single chain antibody
QAVLTQPASLSASPGASASLTCTLRRGINVGAYSIYWYQQKPGSPPQYLLRYKSDSDKQQGSGVSSRFSASKDASANAGILLISGLQSEDEADYYCMIWHSGASAVFGGGTKLTVLGGGGSGGGGSGGGGSEVQLVESGGGLVQPGRSLRLSCAASGFTVSSYWMHWVRQAPGKGLEWVGFIRNKANGGTTEYAASVKGRFTISRDDSKNTLYLQMNSLRAEDTAVYYCARDRGLRFYFDYWGQGTTVTVSSGGGGSDVQLVQSGAEVKKPGASVKVSCKASGYTFTRYTMHWVRQAPGQGLEWIGYINPSRGYTNYADSVKGRFTITTDKSTSTAYMELSSLRSEDTATYYCARYYDDHYCLDYWGQGTTVTVSSGEGTSTGSGGSGGSGGADDIVLTQSPATLSLSPGERATLSCRASQSVSYMNWYQQKPGKAPKRWIYDTSKVASGVPARFSGSGSGTDYSLTINSLEAEDAATYYCQQWSSNPLTFGGGTKVEIK

SEQ ID NO: 17 - protein, 10 aa, Mus musculus
  misc_feature (1)..(10): CDR-H3* A5B7 Shorter CDR-3 of VH of A5B7 with D corresponding to Kabat position 95; Kabat positions 100, 100a, 100b, 101, 102 correspond to FYFDY, respectively
DXXXXFYFDY

SEQ ID NO: 18 - protein, 119 aa, Homo sapiens
EVQLVESGGGVVQPGRSLRLSCAASGFTLSTYAMHWVRQAPGKGLEWVALISNDGSNKYYADSVKGRFTISRDNSKNTLYLQMNSLRAEDTAVYYCARDRGLRFYFDYWGQGTTVTVSS

SEQ ID NO: 19 - protein, 116 aa, Homo sapiens
QAVLTQPASLSASPGASASLTCTLRRGINVGAYSIYWYQQKPGSPPQYLLRYKSDSDKQQGSGVSSRFSASKDASANAGILLISGLQSEDEADYYCMIWHSGASAVFGGGTKLTVL

SEQ ID NO: 20 - protein, 250 aa, Homo sapiens
EVQLVESGGGVVQPGRSLRLSCAASGFTLSTYAMHWVRQAPGKGLEWVALISNDGSNKYYADSVKGRFTISRDNSKNTLYLQMNSLRAEDTAVYYCARDRGLRFYFDYWGQGTTVTVSSGGGGSGGGGSGGGGSQAVLTQPASLSASPGASASLTCTLRRGINVGAYSIYWYQQKPGSPPQYLLRYKSDSDKQQGSGVSSRFSASKDASANAGILLISGLQSEDEADYYCMIWHSGASAVFGGGTKLTVL

SEQ ID NO: 21 - protein, 250 aa, Homo sapiens
QAVLTQPASLSASPGASASLTCTLRRGINVGAYSIYWYQQKPGSPPQYLLRYKSDSDKQQGSGVSSRFSASKDASANAGILLISGLQSEDEADYYCMIWHSGASAVFGGGTKLTVLGGGGSGGGGSGGGGSEVQLVESGGGVVQPGRSLRLSCAASGFTLSTYAMHWVRQAPGKGLEWVALISNDGSNKYYADSVKGRFTISRDNSKNTLYLQMNSLRAEDTAVYYCARDRGLRFYFDYWGQGTTVTVSS

SEQ ID NO: 22 - protein, 119 aa, Homo sapiens
EVQLVESGGGVVQPGRSLRLSCAASGFTLSTYAMHWVRQAPGKGLEWVALISNDGSNKYYADSVKGRFTISRDNSKNTLYLQMNSLRAEDTAVYYCTRDRGLRFYFDYWGQGTTVTVSS

SEQ ID NO: 23 - protein, 250 aa, Homo sapiens
EVQLVESGGGVVQPGRSLRLSCAASGFTLSTYAMHWVRQAPGKGLEWVALISNDGSNKYYADSVKGRFTISRDNSKNTLYLQMNSLRAEDTAVYYCTRDRGLRFYFDYWGQGTTVTVSSGGGGSGGGGSGGGGSQAVLTQPASLSASPGASASLTCTLRRGINVGAYSIYWYQQKPGSPPQYLLRYKSDSDKQQGSGVSSRFSASKDASANAGILLISGLQSEDEADYYCMIWHSGASAVFGGGTKLTVL

SEQ ID NO: 24 - protein, 250 aa, Homo sapiens
QAVLTQPASLSASPGASASLTCTLRRGINVGAYSIYWYQQKPGSPPQYLLRYKSDSDKQQGSGVSSRFSASKDASANAGILLISGLQSEDEADYYCMIWHSGASAVFGGGTKLTVLGGGGSGGGGSGGGGSEVQLVESGGGVVQPGRSLRLSCAASGFTLSTYAMHWVRQAPGKGLEWVALISNDGSNKYYADSVKGRFTISRDNSKNTLYLQMNSLRAEDTAVYYCTRDRGLRFYFDYWGQGTTVTVSS

SEQ ID NO: 25 - protein, 121 aa, Mus musculus
EVQLVESGGGLVQPGGSLRLSCATSGFTFTDYYMNWVRQPPGKALEWLGFIGNKANGYTTEYSASVKGRFTISRDKSQSILYLQMNTLRAEDSATYYCTRDRGLRFYFDYWGQGTTVTVSS

SEQ ID NO: 26 - protein, 252 aa, Artificial sequence
  A5B7 VH - A240 VL ; Single chain Fv
EVQLVESGGGLVQPGGSLRLSCATSGFTFTDYYMNWVRQPPGKALEWLGFIGNKANGYTTEYSASVKGRFTISRDKSQSILYLQMNTLRAEDSATYYCTRDRGLRFYFDYWGQGTTVTVSSGGGGSGGGGSGGGGSQAVLTQPASLSASPGASASLTCTLRRGINVGAYSIYWYQQKPGSPPQYLLRYKSDSDKQQGSGVSSRFSASKDASANAGILLISGLQSEDEADYYCMIWHSGASAVFGGGTKLTVL

SEQ ID NO: 27 - protein, 252 aa, Artificial sequence
  A240 VL -A5B7 VH ; single chain Fv
QAVLTQPASLSASPGASASLTCTLRRGINVGAYSIYWYQQKPGSPPQYLLRYKSDSDKQQGSGVSSRFSASKDASANAGILLISGLQSEDEADYYCMIWHSGASAVFGGGTKLTVLGGGGSGGGGSGGGGSEVQLVESGGGLVQPGGSLRLSCATSGFTFTDYYMNWVRQPPGKALEWLGFIGNKANGYTTEYSASVKGRFTISRDKSQSILYLQMNTLRAEDSATYYCTRDRGLRFYFDYWGQGTTVTVSS

SEQ ID NO: 28 - protein, 121 aa, Homo sapiens
EVQLVESGGGLVQPGRSLRLSCAASGFTVSSYWMHWVRQAPGKGLEWVGFIRNKANGGTTEYAASVKGRFTISRDDSKNTLYLQMNSLRAEDTAVYYCARDRGLRFYFDYWGQGTTVTVSS

SEQ ID NO: 29 - protein, 121 aa, Homo sapiens
EVQLVESGGGLVQPGRSLRLSCAASGFTVSSYWMHWVRQAPGKGLEWVGFILNKANGGTTEYAASVKGRFTISRDDSKNTLYLQMNSLRAEDTAVYYCARDRGLRFYFDYWGQGTTVTVSS

SEQ ID NO: 30 - protein, 490 aa, Artificial sequence
  Bispecific single chain antibody
DIELTQSPAILSASPGEKVTMTCRASSSVTYIHWYQQKPGSSPKSWIYATSNLASGVPARFSGSGSGTSYSLTISRVEAEDAATYYCQHWSSKPPTFGGGTKLEIKGGGGSGGGGSGGGGSQVQLQESGGGLVQPGGSLRLSCATSGFTFTDYYMNWVRQPPGKALEWLGFIGNKANGYTTEYSASVKGRFTISRDKSQSILYLQMNTLRAEDSATYYCTRDRGLRFYFDYWGQGTTVTVSSGGGGSDVQLVQSGAEVKKPGASVKVSCKASGYTFTRYTMHWVRQAPGQGLEWIGYINPSRGYTNYADSVKGRFTITTDKSTSTAYMELSSLRSEDTATYYCARYYDDHYCLDYWGQGTTVTVSSGEGTSTGSGGSGGSGGADDIVLTQSPATLSLSPGERATLSCRASQSVSYMNWYQQKPGKAPKRWIYDTSKVASGVPARFSGSGSGTDYSLTINSLEAEDAATYYCQQWSSNPLTFGGGTKVEIK

SEQ ID NO: 31 - protein, 491 aa, Artificial sequence
  Bispecific single chain antibody
QVQLQESGGGLVQPGGSLRLSCATSGFTFTDYYMNWVRQPPGKALEWLGFIGNKANGYTTEYSASVKGRFTISRDKSQSILYLQMNTLRAEDSATYYCTRDRGLRFYFDYWGQGTTVTVSSGGGGSGGGGSGGGGSDIELTQSPAILSASPGEKVTMTCRASSSVTYIHWYQQKPGSSPKSWIYATSNLASGVPARFSGSGSGTSYSLTISRVEAEDAATYYCQHWSSKPPTFGGGTKLEIKSGGGGSDVQLVQSGAEVKKPGASVKVSCKASGYTFTRYTMHWVRQAPGQGLEWIGYINPSRGYTNYADSVKGRFTITTDKSTSTAYMELSSLRSEDTATYYCARYYDDHYCLDYWGQGTTVTVSSGEGTSTGSGGSGGSGGADDIVLTQSPATLSLSPGERATLSCRASQSVSYMNWYQQKPGKAPKRWIYDTSKVASGVPARFSGSGSGTDYSLTINSLEAEDAATYYCQQWSSNPLTFGGGTKVEIK

SEQ ID NO: 32 - protein, 501 aa, Artificial sequence
  Bispecific single chain antibody
EVQLVESGGGLVQPGGSLRLSCATSGFTFTDYYMNWVRQPPGKALEWLGFIGNKANGYTTEYSASVKGRFTISRDKSQSILYLQMNTLRAEDSATYYCTRDRGLRFYFDYWGQGTTVTVSSGGGGSGGGGSGGGGSQAVLTQPASLSASPGASASLTCTLRRGINVGAYSIYWYQQKPGSPPQYLLRYKSDSDKQQGSGVSSRFSASKDASANAGILLISGLQSEDEADYYCMIWHSGASAVFGGGTKLTVLSGGGGSDVQLVQSGAEVKKPGASVKVSCKASGYTFTRYTMHWVRQAPGQGLEWIGYINPSRGYTNYADSVKGRFTITTDKSTSTAYMELSSLRSEDTATYYCARYYDDHYCLDYWGQGTTVTVSSGEGTSTGSGGSGGSGGADDIVLTQSPATLSLSPGERATLSCRASQSVSYMNWYQQKPGKAPKRWIYDTSKVASGVPARFSGSGSGTDYSLTINSLEAEDAATYYCQQWSSNPLTFGGGTKVEIK

SEQ ID NO: 33 - protein, 500 aa, Artificial sequence
  Bispecific single chain antibody
QAVLTQPASLSASPGASASLTCTLRRGINVGAYSIYWYQQKPGSPPQYLLRYKSDSDKQQGSGVSSRFSASKDASANAGILLISGLQSEDEADYYCMIWHSGASAVFGGGTKLTVLGGGGSGGGGSGGGGSEVQLVESGGGLVQPGGSLRLSCATSGFTFTDYYMNWVRQPPGKALEWLGFIGNKANGYTTEYSASVKGRFTISRDKSQSILYLQMNTLRAEDSATYYCTRDRGLRFYFDYWGQGTTVTVSSGGGGSDVQLVQSGAEVKKPGASVKVSCKASGYTFTRYTMHWVRQAPGQGLEWIGYINPSRGYTNYADSVKGRFTITTDKSTSTAYMELSSLRSEDTATYYCARYYDDHYCLDYWGQGTTVTVSSGEGTSTGSGGSGGSGGADDIVLTQSPATLSLSPGERATLSCRASQSVSYMNWYQQKPGKAPKRWIYDTSKVASGVPARFSGSGSGTDYSLTINSLEAEDAATYYCQQWSSNPLTFGGGTKVEIK

SEQ ID NO: 34 - protein, 499 aa, Artificial sequence
  Bispecific single chain antibody
EVQLVESGGGVVQPGRSLRLSCAASGFTLSTYAMHWVRQAPGKGLEWVALISNDGSNKYYADSVKGRFTISRDNSKNTLYLQMNSLRAEDTAVYYCARDRGLRFYFDYWGQGTTVTVSSGGGGSGGGGSGGGGSELVLTQPASLSASPGASASLTCTLRRGINVGAYSIYWYQQKPGSPPQYLLRYKSDSDKQQGSGVSSRFSASKDASANAGILLISGLQSEDEADYYCMIWHSGASAVFGGGTKLTVLSGGGGSDVQLVQSGAEVKKPGASVKVSCKASGYTFTRYTMHWVRQAPGQGLEWIGYINPSRGYTNYADSVKGRFTITTDKSTSTAYMELSSLRSEDTATYYCARYYDDHYCLDYWGQGTTVTVSSGEGTSTGSGGSGGSGGADDIVLTQSPATLSLSPGERATLSCRASQSVSYMNWYQQKPGKAPKRWIYDTSKVASGVPARFSGSGSGTDYSLTINSLEAEDAATYYCQQWSSNPLTFGGGTKVEIK

SEQ ID NO: 35 - protein, 498 aa, Artificial sequence
  Bispecific single chain antibody
QAVLTQPASLSASPGASASLTCTLRRGINVGAYSIYWYQQKPGSPPQYLLRYKSDSDKQQGSGVSSRFSASKDASANAGILLISGLQSEDEADYYCMIWHSGASAVFGGGTKLTVLGGGGSGGGGSGGGGSEVQLVESGGGVVQPGRSLRLSCAASGFTLSTYAMHWVRQAPGKGLEWVALISNDGSNKYYADSVKGRFTISRDNSKNTLYLQMNSLRAEDTAVYYCARDRGLRFYFDYWGQGTTVTVSSGGGGSDVQLVQSGAEVKKPGASVKVSCKASGYTFTRYTMHWVRQAPGQGLEWIGYINPSRGYTNYADSVKGRFTITTDKSTSTAYMELSSLRSEDTATYYCARYYDDHYCLDYWGQGTTVTVSSGEGTSTGSGGSGGSGGADDIVLTQSPATLSLSPGERATLSCRASQSVSYMNWYQQKPGKAPKRWIYDTSKVASGVPARFSGSGSGTDYSLTINSLEAEDAATYYCQQWSSNPLTFGGGTKVEIK

SEQ ID NO: 36 - protein, 501 aa, Artificial sequence
  Bispecific single chain antibody
EVQLVESGGGLVQPGRSLRLSCAASGFTVSSYWMHWVRQAPGKGLEWVGFIRNKANGGTTEYAASVKGRFTISRDDSKNTLYLQMNSLRAEDTAVYYCARDRGLRFYFDYWGQGTTVTVSSGGGGSGGGGSGGGGSELVLTQPASLSASPGASASLTCTLRRGINVGAYSIYWYQQKPGSPPQYLLRYKSDSDKQQGSGVSSRFSASKDASANAGILLISGLQSEDEADYYCMIWHSGASAVFGGGTKLTVLSGGGGSDVQLVQSGAEVKKPGASVKVSCKASGYTFTRYTMHWVRQAPGQGLEWIGYINPSRGYTNYADSVKGRFTITTDKSTSTAYMELSSLRSEDTATYYCARYYDDHYCLDYWGQGTTVTVSSGEGTSTGSGGSGGSGGADDIVLTQSPATLSLSPGERATLSCRASQSVSYMNWYQQKPGKAPKRWIYDTSKVASGVPARFSGSGSGTDYSLTINSLEAEDAATYYCQQWSSNPLTFGGGTKVEIK

SEQ ID NO: 37 - protein, 500 aa, Artificial sequence; identical to SEQ ID NO: 16
  Bispecific single chain antibody

SEQ ID NO: 38 - protein, 499 aa, Artificial sequence
  Bispecific single chain antibody
EVQLVESGGGVVQPGRSLRLSCAASGFTLSTYAMHWVRQAPGKGLEWVALISNDGSNKYYADSVKGRFTISRDNSKNTLYLQMNSLRAEDTAVYYCTRDRGLRFYFDYWGQGTTVTVSSGGGGSGGGGSGGGGSELVLTQPASLSASPGASASLTCTLRRGINVGAYSIYWYQQKPGSPPQYLLRYKSDSDKQQGSGVSSRFSASKDASANAGILLISGLQSEDEADYYCMIWHSGASAVFGGGTKLTVLSGGGGSDVQLVQSGAEVKKPGASVKVSCKASGYTFTRYTMHWVRQAPGQGLEWIGYINPSRGYTNYADSVKGRFTITTDKSTSTAYMELSSLRSEDTATYYCARYYDDHYCLDYWGQGTTVTVSSGEGTSTGSGGSGGSGGADDIVLTQSPATLSLSPGERATLSCRASQSVSYMNWYQQKPGKAPKRWIYDTSKVASGVPARFSGSGSGTDYSLTINSLEAEDAATYYCQQWSSNPLTFGGGTKVEIK

SEQ ID NO: 39 - protein, 498 aa, Artificial sequence
  Bispecific single chain antibody
QAVLTQPASLSASPGASASLTCTLRRGINVGAYSIYWYQQKPGSPPQYLLRYKSDSDKQQGSGVSSRFSASKDASANAGILLISGLQSEDEADYYCMIWHSGASAVFGGGTKLTVLGGGGSGGGGSGGGGSEVQLVESGGGVVQPGRSLRLSCAASGFTLSTYAMHWVRQAPGKGLEWVALISNDGSNKYYADSVKGRFTISRDNSKNTLYLQMNSLRAEDTAVYYCTRDRGLRFYFDYWGQGTTVTVSSGGGGSDVQLVQSGAEVKKPGASVKVSCKASGYTFTRYTMHWVRQAPGQGLEWIGYINPSRGYTNYADSVKGRFTITTDKSTSTAYMELSSLRSEDTATYYCARYYDDHYCLDYWGQGTTVTVSSGEGTSTGSGGSGGSGGADDIVLTQSPATLSLSPGERATLSCRASQSVSYMNWYQQKPGKAPKRWIYDTSKVASGVPARFSGSGSGTDYSLTINSLEAEDAATYYCQQWSSNPLTFGGGTKVEIK

SEQ ID NO: 40 - protein, 499 aa, Artificial sequence
  Bispecific single chain antibody
EVQLVESGGGVVQPGRSLRLSCAASGFTLSTYAMHWVRQAPGKGLEWVALISNDGSNKYYADSVKGRFTISRDNSKNTLYLQMNSLRAEDTAVYYCARDRGLRFYFDYWGQGTTVTVSSGGGGSGGGGSGGGGSQAVLTQPASLSASPGASASLTCTLRRGINVGAYSIYWYQQKPGSPPQYLLRYKSDSDKQQGSGVSSRFSASKDASANAGILLISGLQSEDEADYYCMIWHSGASAVFGGGTKLTVLSGGGGSDVQLVQSGAEVKKPGASVKVSCKASGYTFTRYTMHWVRQAPGQGLEWIGYINPSRGYTNYADSVKGRFTITTDKSTSTAYMELSSLRSEDTATYYCARYYDDHYCLDYWGQGTTVTVSSGEGTSTGSGGSGGSGGADDIVLTQSPATLSLSPGERATLSCRASQSVSYMNWYQQKPGKAPKRWIYDTSKVASGVPARFSGSGSGTDYSLTINSLEAEDAATYYCQQWSSNPLTFGGGTKVEIK

SEQ ID NO: 41 - protein, 501 aa, Artificial sequence
  Bispecific single chain antibody
EVQLVESGGGLVQPGRSLRLSCAASGFTVSSYWMHWVRQAPGKGLEWVGFIRNKANGGTTEYAASVKGRFTISRDDSKNTLYLQMNSLRAEDTAVYYCARDRGLRFYFDYWGQGTTVTVSSGGGGSGGGGSGGGGSQAVLTQPASLSASPGASASLTCTLRRGINVGAYSIYWYQQKPGSPPQYLLRYKSDSDKQQGSGVSSRFSASKDASANAGILLISGLQSEDEADYYCMIWHSGASAVFGGGTKLTVLSGGGGSDVQLVQSGAEVKKPGASVKVSCKASGYTFTRYTMHWVRQAPGQGLEWIGYINPSRGYTNYADSVKGRFTITTDKSTSTAYMELSSLRSEDTATYYCARYYDDHYCLDYWGQGTTVTVSSGEGTSTGSGGSGGSGGADDIVLTQSPATLSLSPGERATLSCRASQSVSYMNWYQQKPGKAPKRWIYDTSKVASGVPARFSGSGSGTDYSLTINSLEAEDAATYYCQQWSSNPLTFGGGTKVEIK

SEQ ID NO: 42 - protein, 499 aa, Artificial sequence
  Bispecific single chain antibody
EVQLVESGGGVVQPGRSLRLSCAASGFTLSTYAMHWVRQAPGKGLEWVALISNDGSNKYYADSVKGRFTISRDNSKNTLYLQMNSLRAEDTAVYYCTRDRGLRFYFDYWGQGTTVTVSSGGGGSGGGGSGGGGSQAVLTQPASLSASPGASASLTCTLRRGINVGAYSIYWYQQKPGSPPQYLLRYKSDSDKQQGSGVSSRFSASKDASANAGILLISGLQSEDEADYYCMIWHSGASAVFGGGTKLTVLSGGGGSDVQLVQSGAEVKKPGASVKVSCKASGYTFTRYTMHWVRQAPGQGLEWIGYINPSRGYTNYADSVKGRFTITTDKSTSTAYMELSSLRSEDTATYYCARYYDDHYCLDYWGQGTTVTVSSGEGTSTGSGGSGGSGGADDIVLTQSPATLSLSPGERATLSCRASQSVSYMNWYQQKPGKAPKRWIYDTSKVASGVPARFSGSGSGTDYSLTINSLEAEDAATYYCQQWSSNPLTFGGGTKVEIK

SEQ ID NO: 43 - protein, 507 aa, Artificial sequence
  Bispecific single chain antibody
DVQLVQSGAEVKKPGASVKVSCKASGYTFTRYTMHWVRQAPGQGLEWIGYINPSRGYTNYADSVKGRFTITTDKSTSTAYMELSSLRSEDTATYYCARYYDDHYCLDYWGQGTTVTVSSGEGTSTGSGGSGGSGGADDIVLTQSPATLSLSPGERATLSCRASQSVSYMNWYQQKPGKAPKRWIYDTSKVASGVPARFSGSGSGTDYSLTINSLEAEDAATYYCQQWSSNPLTFGGGTKVEIKSGGGGSEVQLVESGGGLVQPGRSLRLSCAASGFTVSSYWMHWVRQAPGKGLEWVGFILNKANGGTTEYAASVKGRFTISRDDSKNTLYLQMNSLRAEDTAVYYCARDRGLRFYFDYWGQGTTVTVSSGGGGSGGGGSGGGGSELVLTQPASLSASPGASASLTCTLRRGINVGAYSIYWYQQKPGSPPQYLLRYKSDSDKQQGSGVSSRFSASKDASANAGILLISGLQSEDEADYYCMIWHSGASAVFGGGTKLTVLHHHHHH

SEQ ID NO: 44 - DNA, 1473 bp, Artificial sequence
  Bispecific single-chain antibody
gacattgagctcacccagtctccagcaatcctgtctgcatctccaggggagaaggtcacaatgacttgcagggccagctcaagtgtaacttacattcactggtaccagcagaagccaggatcctcccccaaatcctggatttatgccacatccaacctggcttctggagtccctgctcgcttcagtggcagtgggtctgggacctcttactctctcacaatcagcagagtggaggctgaagatgctgccacttattactgccaacattggagtagtaaaccaccgacgttcggtggagggaccaagctcgagatcaaaggtggtggtggttctggcggcggcggctccggtggtggtggttctcaggtccaactgcaggagtcaggaggaggcttggtacagcctgggggttctctgagactctcctgtgcaacttctgggttcaccttcactgattactacatgaactgggtccgccagcctccaggaaaggcacttgagtggttgggttttattggaaacaaagctaatggttacacaacagagtacagtgcatctgtgaagggtcggttcaccatctccagagataaatcccaaagcatcctctatcttcaaatgaacaccctgagagctgaggacagtgccacttattactgtaccagagatagggggctacggttctactttgactactggggccaagggaccacggtcaccgtctcctccggaggtggtggatccgacgtccaactggtgcagtcaggggctgaagtgaaaaaacctggggcctcagtgaaggtgtcctgcaaggcttctggctacacctttactaggtacacgatgcactgggtaaggcaggcacctggacagggtctggaatggattggatacattaatcctagccgtggttatactaattacgcagacagcgtcaagggccgcttcacaatcactacagacaaatccaccagcacagcctacatggaactgagcagcctgcgttctgaggacactgcaacctattactgtgcaagatattatgatgatcattactgccttgactactggggccaaggcaccacggtcaccgtctcctcaggcgaaggtactagtactggttctggtggaagtggaggttcaggtggagcagacgacattgtactgacccagtctccagcaactctgtctctgtctccaggggagcgtgccaccctgagctgcagagccagtcaaagtgtaagttacatgaactggtaccagcagaagccgggcaaggcacccaaaagatggatttatgacacatccaaagtggcttctggagtccctgctcgcttcagtggcagtgggtctgggaccgactactctctcacaatcaacagcttggaggctgaagatgctgccacttattactgccaacagtggagtagtaacccgctcacgttcggtggcgggaccaaggtggagatcaaatag

SEQ ID NO: 45 - DNA, 1476 bp, Artificial sequence
  Bispecific single chain antibody
caggtccaactgcaggagtcaggaggaggcttggtacagcctgggggttctctgagactctcctgtgcaacttctgggttcaccttcactgattactacatgaactgggtccgccagcctccaggaaaggcacttgagtggttgggttttattggaaacaaagctaatggttacacaacagagtacagtgcatctgtgaagggtcggttcaccatctccagagataaatcccaaagcatcctctatcttcaaatgaacaccctgagagctgaggacagtgccacttattactgtaccagagatagggggctacggttctactttgactactggggccaagggaccacggtcaccgtctcctccggtggtggtggttctggcggcggcggctccggtggtggtggttctgacattgagctcacccagtctccagcaatcctgtctgcatctccaggggagaaggtcacaatgacttgcagggccagctcaagtgtaacttacattcactggtaccagcagaagccaggatcctcccccaaatcctggatttatgccacatccaacctggcttctggagtccctgctcgcttcagtggcagtgggtctgggacctcttactctctcacaatcagcagagtggaggctgaagatgctgccacttattactgccaacattggagtagtaaaccaccgacgttcggtggagggaccaagctcgagatcaaatccggaggtggtggatccgacgtccaactggtgcagtcaggggctgaagtgaaaaaacctggggcctcagtgaaggtgtcctgcaaggcttctggctacacctttactaggtacacgatgcactgggtaaggcaggcacctggacagggtctggaatggattggatacattaatcctagccgtggttatactaattacgcagacagcgtcaagggccgcttcacaatcactacagacaaatccaccagcacagcctacatggaactgagcagcctgcgttctgaggacactgcaacctattactgtgcaagatattatgatgatcattactgccttgactactggggccaaggcaccacggtcaccgtctcctcaggcgaaggtactagtactggttctggtggaagtggaggttcaggtggagcagacgacattgtactgacccagtctccagcaactctgtctctgtctccaggggagcgtgccaccctgagctgcagagccagtcaaagtgtaagttacatgaactggtaccagcagaagccgggcaaggcacccaaaagatggatttatgacacatccaaagtggcttctggagtccctgctcgcttcagtggcagtgggtctgggaccgactactctctcacaatcaacagcttggaggctgaagatgctgccacttattactgccaacagtggagtagtaacccgctcacgttcggtggcgggaccaaggtggagatcaaatag

SEQ ID NO: 46 - DNA, 1506 bp, Artificial sequence
  Bispecific single chain antibody
gaggtgcagctggtcgagtcaggaggaggcttggtacagcctgggggttctctgagactctcctgtgcaacttctgggttcaccttcactgattactacatgaactgggtccgccagcctccaggaaaggcacttgagtggttgggttttattggaaacaaagctaatggttacacaacagagtacagtgcatctgtgaagggtcggttcaccatctccagagataaatcccaaagcatcctctatcttcaaatgaacaccctgagagctgaggacagtgccacttattactgtaccagagatagggggctacggttctactttgactactggggccaagggaccacggtcaccgtctcctcaggtggtggtggttctggcggcggcggctccggtggtggtggttctcaggccgtgctgactcagccggcttccctctctgcatctcctggagcatcagccagtctcacctgcaccttgcgcaggggcatcaatgttggtgcctacagtatatactggtaccagcagaagccagggagtcctccccagtatctcctgaggtacaaatcagactcagataagcagcagggctctggagtctccagccgcttctctgcatccaaagatgcttcggccaatgcagggattttactcatctctgggctccagtctgaggatgaggctgactattactgtatgatttggcacagcggcgcttctgcggtgttcggcggagggaccaagttgaccgtcctatccggaggtggtggatccgacgtccaactggtgcagtcaggggctgaagtgaaaaaacctggggcctcagtgaaggtgtcctgcaaggcttctggctacacctttactaggtacacgatgcactgggtaaggcaggcacctggacagggtctggaatggattggatacattaatcctagccgtggttatactaattacgcagacagcgtcaagggccgcttcacaatcactacagacaaatccaccagcacagcctacatggaactgagcagcctgcgttctgaggacactgcaacctattactgtgcaagatattatgatgatcattactgccttgactactggggccaaggcaccacggtcaccgtctcctcaggcgaaggtactagtactggttctggtggaagtggaggttcaggtggagcagacgacattgtactgacccagtctccagcaactctgtctctgtctccaggggagcgtgccaccctgagctgcagagccagtcaaagtgtaagttacatgaactggtaccagcagaagccgggcaaggcacccaaaagatggatttatgacacatccaaagtggcttctggagtccctgctcgcttcagtggcagtgggtctgggaccgactactctctcacaatcaacagcttggaggctgaagatgctgccacttattactgccaacagtggagtagtaacccgctcacgttcggtggcgggaccaaggtggagatcaaatag

SEQ ID NO: 47 - DNA, 1503 bp, Artificial sequence
  Bispecific single chain antibody
caggccgtgctgactcagccggcttccctctctgcatctcctggagcatcagccagtctcacctgcaccttgcgcaggggcatcaatgttggtgcctacagtatatactggtaccagcagaagccagggagtcctccccagtatctcctgaggtacaaatcagactcagataagcagcagggctctggagtctccagccgcttctctgcatccaaagatgcttcggccaatgcagggattttactcatctctgggctccagtctgaggatgaggctgactattactgtatgatttggcacagcggcgcttctgcggtgttcggcggagggaccaagttgaccgtcctaggtggtggtggttctggcggcggcggctccggtggtggtggttctgaggtgcagctggtcgagtcaggaggaggcttggtacagcctgggggttctctgagactctcctgtgcaacttctgggttcaccttcactgattactacatgaactgggtccgccagcctccaggaaaggcacttgagtggttgggttttattggaaacaaagctaatggttacacaacagagtacagtgcatctgtgaagggtcggttcaccatctccagagataaatcccaaagcatcctctatcttcaaatgaacaccctgagagctgaggacagtgccacttattactgtaccagagatagggggctacggttctactttgactactggggccaagggaccacggtcaccgtctcctccggaggtggtggatccgacgtccaactggtgcagtcaggggctgaagtgaaaaaacctggggcctcagtgaaggtgtcctgcaaggcttctggctacacctttactaggtacacgatgcactgggtaaggcaggcacctggacagggtctggaatggattggatacattaatcctagccgtggttatactaattacgcagacagcgtcaagggccgcttcacaatcactacagacaaatccaccagcacagcctacatggaactgagcagcctgcgttctgaggacactgcaacctattactgtgcaagatattatgatgatcattactgccttgactactggggccaaggcaccacggtcaccgtctcctcaggcgaaggtactagtactggttctggtggaagtggaggttcaggtggagcagacgacattgtactgacccagtctccagcaactctgtctctgtctccaggggagcgtgccaccctgagctgcagagccagtcaaagtgtaagttacatgaactggtaccagcagaagccgggcaaggcacccaaaagatggatttatgacacatccaaagtggcttctggagtccctgctcgcttcagtggcagtgggtctgggaccgactactctctcacaatcaacagcttggaggctgaagatgctgccacttattactgccaacagtggagtagtaacccgctcacgttcggtggcgggaccaaggtggagatcaaatag

SEQ ID NO: 48 - DNA, 1500 bp, Artificial sequence
  Bispecific single chain antibody
gaggtgcagctggtcgagtctgggggaggcgtggtccagcctgggaggtccctgagactctcctgtgcagcctctggattcaccctcagtacctatgccatgcactgggtccgccaggctccaggcaaggggctggagtgggtggcacttatatcaaatgatggaagcaataaatactatgcagactccgtgaagggccgattcaccatctccagagacaattccaagaacacgctgtatctgcaaatgaacagcctgagagctgaggacacggccgtgtattactgtgcgagagatagggggctacggttctactttgactactggggccaagggaccacggtcaccgtctcctcaggtggtggtggttctggcggcggcggctccggtggtggtggttctgagctcgtgctgactcagccggcttccctctctgcatctcctggagcatcagccagtctcacctgcaccttgcgcaggggcatcaatgttggtgcctacagtatatactggtaccagcagaagccagggagtcctccccagtatctcctgaggtacaaatcagactcagataagcagcagggctctggagtctccagccgcttctctgcatccaaagatgcttcggccaatgcagggattttactcatctctgggctccagtctgaggatgaggctgactattactgtatgatttggcacagcggcgcttctgcggtgttcggcggagggaccaagttgaccgtcctatccggaggtggtggatccgacgtccaactggtgcagtcaggggctgaagtgaaaaaacctggggcctcagtgaaggtgtcctgcaaggcttctggctacacctttactaggtacacgatgcactgggtaaggcaggcacctggacagggtctggaatggattggatacattaatcctagccgtggttatactaattacgcagacagcgtcaagggccgcttcacaatcactacagacaaatccaccagcacagcctacatggaactgagcagcctgcgttctgaggacactgcaacctattactgtgcaagatattatgatgatcattactgccttgactactggggccaaggcaccacggtcaccgtctcctcaggcgaaggtactagtactggttctggtggaagtggaggttcaggtggagcagacgacattgtactgacccagtctccagcaactctgtctctgtctccaggggagcgtgccaccctgagctgcagagccagtcaaagtgtaagttacatgaactggtaccagcagaagccgggcaaggcacccaaaagatggatttatgacacatccaaagtggcttctggagtccctgctcgcttcagtggcagtgggtctgggaccgactactctctcacaatcaacagcttggaggctgaagatgctgccacttattactgccaacagtggagtagtaacccgctcacgttcggtggcgggaccaaggtggagatcaaatag

SEQ ID NO: 49 - DNA, 1497 bp, Artificial sequence
  Bispecific single chain antibody
caggccgtgctgactcagccggcttccctctctgcatctcctggagcatcagccagtctcacctgcaccttgcgcaggggcatcaatgttggtgcctacagtatatactggtaccagcagaagccagggagtcctccccagtatctcctgaggtacaaatcagactcagataagcagcagggctctggagtctccagccgcttctctgcatccaaagatgcttcggccaatgcagggattttactcatctctgggctccagtctgaggatgaggctgactattactgtatgatttggcacagcggcgcttctgcggtgttcggcggagggaccaagttgaccgtcctaggtggtggtggttctggcggcggcggctccggtggtggtggttctgaggtgcagctggtcgagtctgggggaggcgtggtccagcctgggaggtccctgagactctcctgtgcagcctctggattcaccctcagtacctatgccatgcactgggtccgccaggctccaggcaaggggctggagtgggtggcacttatatcaaatgatggaagcaataaatactatgcagactccgtgaagggccgattcaccatctccagagacaattccaagaacacgctgtatctgcaaatgaacagcctgagagctgaggacacggccgtgtattactgtgcgagagatagggggctacggttctactttgactactggggccaagggaccacggtcaccgtctcctccggaggtggtggatccgacgtccaactggtgcagtcaggggctgaagtgaaaaaacctggggcctcagtgaaggtgtcctgcaaggcttctggctacacctttactaggtacacgatgcactgggtaaggcaggcacctggacagggtctggaatggattggatacattaatcctagccgtggttatactaattacgcagacagcgtcaagggccgcttcacaatcactacagacaaatccaccagcacagcctacatggaactgagcagcctgcgttctgaggacactgcaacctattactgtgcaagatattatgatgatcattactgccttgactactggggccaaggcaccacggtcaccgtctcctcaggcgaaggtactagtactggttctggtggaagtggaggttcaggtggagcagacgacattgtactgacccagtctccagcaactctgtctctgtctccaggggagcgtgccaccctgagctgcagagccagtcaaagtgtaagttacatgaactggtaccagcagaagccgggcaaggcacccaaaagatggatttatgacacatccaaagtggcttctggagtccctgctcgcttcagtggcagtgggtctgggaccgactactctctcacaatcaacagcttggaggctgaagatgctgccacttattactgccaacagtggagtagtaacccgctcacgttcggtggcgggaccaaggtggagatcaaatag

SEQ ID NO: 50 - DNA, 1506 bp, Artificial sequence
  Bispecific single chain antibody
gaggtgcagctggtcgagtctgggggaggcttggtccagcctgggaggtccctgagactctcctgtgcagcgtctggattcaccgtcagtagctactggatgcactgggtccgccaagctccagggaaggggctggaatgggtaggtttcattagaaacaaagctaatggtgggacaacagaatacgccgcgtctgtgaaaggcagattcaccatctcaagagatgattccaagaacacgctgtatcttcaaatgaacagcctgagagccgaggacacggccgtgtattactgtgcaagagatagggggctacggttctactttgactactggggccaagggaccacggtcaccgtctcctcaggtggtggtggttctggcggcggcggctccggtggtggtggttctgagctcgtgctgactcagccggcttccctctctgcatctcctggagcatcagccagtctcacctgcaccttgcgcaggggcatcaatgttggtgcctacagtatatactggtaccagcagaagccagggagtcctccccagtatctcctgaggtacaaatcagactcagataagcagcagggctctggagtctccagccgcttctctgcatccaaagatgcttcggccaatgcagggattttactcatctctgggctccagtctgaggatgaggctgactattactgtatgatttggcacagcggcgcttctgcggtgttcggcggagggaccaagttgaccgtcctatccggaggtggtggatccgacgtccaactggtgcagtcaggggctgaagtgaaaaaacctggggcctcagtgaaggtgtcctgcaaggcttctggctacacctttactaggtacacgatgcactgggtaaggcaggcacctggacagggtctggaatggattggatacattaatcctagccgtggttatactaattacgcagacagcgtcaagggccgcttcacaatcactacagacaaatccaccagcacagcctacatggaactgagcagcctgcgttctgaggacactgcaacctattactgtgcaagatattatgatgatcattactgccttgactactggggccaaggcaccacggtcaccgtctcctcaggcgaaggtactagtactggttctggtggaagtggaggttcaggtggagcagacgacattgtactgacccagtctccagcaactctgtctctgtctccaggggagcgtgccaccctgagctgcagagccagtcaaagtgtaagttacatgaactggtaccagcagaagccgggcaaggcacccaaaagatggatttatgacacatccaaagtggcttctggagtccctgctcgcttcagtggcagtgggtctgggaccgactactctctcacaatcaacagcttggaggctgaagatgctgccacttattactgccaacagtggagtagtaacccgctcacgttcggtggcgggaccaaggtggagatcaaatag

SEQ ID NO: 51 - DNA, 1503 bp, Artificial sequence
  Bispecific single chain antibody
caggccgtgctgactcagccggcttccctctctgcatctcctggagcatcagccagtctcacctgcaccttgcgcaggggcatcaatgttggtgcctacagtatatactggtaccagcagaagccagggagtcctccccagtatctcctgaggtacaaatcagactcagataagcagcagggctctggagtctccagccgcttctctgcatccaaagatgcttcggccaatgcagggattttactcatctctgggctccagtctgaggatgaggctgactattactgtatgatttggcacagcggcgcttctgcggtgttcggcggagggaccaagttgaccgtcctaggtggtggtggttctggcggcggcggctccggtggtggtggttctgaggtgcagctggtcgagtctgggggaggcttggtccagcctgggaggtccctgagactctcctgtgcagcgtctggattcaccgtcagtagctactggatgcactgggtccgccaagctccagggaaggggctggaatgggtaggtttcattagaaacaaagctaatggtgggacaacagaatacgccgcgtctgtgaaaggcagattcaccatctcaagagatgattccaagaacacgctgtatcttcaaatgaacagcctgagagccgaggacacggccgtgtattactgtgcaagagatagggggctacggttctactttgactactggggccaagggaccacggtcaccgtctcctccggaggtggtggatccgacgtccaactggtgcagtcaggggctgaagtgaaaaaacctggggcctcagtgaaggtgtcctgcaaggcttctggctacacctttactaggtacacgatgcactgggtaaggcaggcacctggacagggtctggaatggattggatacattaatcctagccgtggttatactaattacgcagacagcgtcaagggccgcttcacaatcactacagacaaatccaccagcacagcctacatggaactgagcagcctgcgttctgaggacactgcaacctattactgtgcaagatattatgatgatcattactgccttgactactggggccaaggcaccacggtcaccgtctcctcaggcgaaggtactagtactggttctggtggaagtggaggttcaggtggagcagacgacattgtactgacccagtctccagcaactctgtctctgtctccaggggagcgtgccaccctgagctgcagagccagtcaaagtgtaagttacatgaactggtaccagcagaagccgggcaaggcacccaaaagatggatttatgacacatccaaagtggcttctggagtccctgctcgcttcagtggcagtgggtctgggaccgactactctctcacaatcaacagcttggaggctgaagatgctgccacttattactgccaacagtggagtagtaacccgctcacgttcggtggcgggaccaaggtggagatcaaatag

SEQ ID NO: 52 - DNA, 1500 bp, Artificial sequence
  Bispecific single chain antibody
gaggtgcagctggtcgagtctgggggaggcgtggtccagcctgggaggtccctgagactctcctgtgcagcctctggattcaccctcagtacctatgccatgcactgggtccgccaggctccaggcaaggggctggagtgggtggcacttatatcaaatgatggaagcaataaatactatgcagactccgtgaagggccgattcaccatctccagagacaattccaagaacacgctgtatctgcaaatgaacagcctgagagctgaggacacggctgtgtattactgtactagagatagggggctacggttctactttgactactggggccaagggaccacggtcaccgtctcctcaggtggtggtggttctggcggcggcggctccggtggtggtggttctgagctcgtgctgactcagccggcttccctctctgcatctcctggagcatcagccagtctcacctgcaccttgcgcaggggcatcaatgttggtgcctacagtatatactggtaccagcagaagccagggagtcctccccagtatctcctgaggtacaaatcagactcagataagcagcagggctctggagtctccagccgcttctctgcatccaaagatgcttcggccaatgcagggattttactcatctctgggctccagtctgaggatgaggctgactattactgtatgatttggcacagcggcgcttctgcggtgttcggcggagggaccaagttgaccgtcctatccggaggtggtggatccgacgtccaactggtgcagtcaggggctgaagtgaaaaaacctggggcctcagtgaaggtgtcctgcaaggcttctggctacacctttactaggtacacgatgcactgggtaaggcaggcacctggacagggtctggaatggattggatacattaatcctagccgtggttatactaattacgcagacagcgtcaagggccgcttcacaatcactacagacaaatccaccagcacagcctacatggaactgagcagcctgcgttctgaggacactgcaacctattactgtgcaagatattatgatgatcattactgccttgactactggggccaaggcaccacggtcaccgtctcctcaggcgaaggtactagtactggttctggtggaagtggaggttcaggtggagcagacgacattgtactgacccagtctccagcaactctgtctctgtctccaggggagcgtgccaccctgagctgcagagccagtcaaagtgtaagttacatgaactggtaccagcagaagccgggcaaggcacccaaaagatggatttatgacacatccaaagtggcttctggagtccctgctcgcttcagtggcagtgggtctgggaccgactactctctcacaatcaacagcttggaggctgaagatgctgccacttattactgccaacagtggagtagtaacccgctcacgttcggtggcgggaccaaggtggagatcaaatag

SEQ ID NO: 53 - DNA, 1497 bp, Artificial sequence
  Bispecific single chain antibody
caggccgtgctgactcagccggcttccctctctgcatctcctggagcatcagccagtctcacctgcaccttgcgcaggggcatcaatgttggtgcctacagtatatactggtaccagcagaagccagggagtcctccccagtatctcctgaggtacaaatcagactcagataagcagcagggctctggagtctccagccgcttctctgcatccaaagatgcttcggccaatgcagggattttactcatctctgggctccagtctgaggatgaggctgactattactgtatgatttggcacagcggcgcttctgcggtgttcggcggagggaccaagttgaccgtcctaggtggtggtggttctggcggcggcggctccggtggtggtggttctgaggtgcagctggtcgagtctgggggaggcgtggtccagcctgggaggtccctgagactctcctgtgcagcctctggattcaccctcagtacctatgccatgcactgggtccgccaggctccaggcaaggggctggagtgggtggcacttatatcaaatgatggaagcaataaatactatgcagactccgtgaagggccgattcaccatctccagagacaattccaagaacacgctgtatctgcaaatgaacagcctgagagctgaggacacggctgtgtattactgtactagagatagggggctacggttctactttgactactggggccaagggaccacggtcaccgtctcctccggaggtggtggatccgacgtccaactggtgcagtcaggggctgaagtgaaaaaacctggggcctcagtgaaggtgtcctgcaaggcttctggctacacctttactaggtacacgatgcactgggtaaggcaggcacctggacagggtctggaatggattggatacattaatcctagccgtggttatactaattacgcagacagcgtcaagggccgcttcacaatcactacagacaaatccaccagcacagcctacatggaactgagcagcctgcgttctgaggacactgcaacctattactgtgcaagatattatgatgatcattactgccttgactactggggccaaggcaccacggtcaccgtctcctcaggcgaaggtactagtactggttctggtggaagtggaggttcaggtggagcagacgacattgtactgacccagtctccagcaactctgtctctgtctccaggggagcgtgccaccctgagctgcagagccagtcaaagtgtaagttacatgaactggtaccagcagaagccgggcaaggcacccaaaagatggatttatgacacatccaaagtggcttctggagtccctgctcgcttcagtggcagtgggtctgggaccgactactctctcacaatcaacagcttggaggctgaagatgctgccacttattactgccaacagtggagtagtaacccgctcacgttcggtggcgggaccaaggtggagatcaaatag

SEQ ID NO: 54 - DNA, 1500 bp, Artificial sequence
  Bispecific single chain antibody
gaggtgcagctggtcgagtctgggggaggcgtggtccagcctgggaggtccctgagactctcctgtgcagcctctggattcaccctcagtacctatgccatgcactgggtccgccaggctccaggcaaggggctggagtgggtggcacttatatcaaatgatggaagcaataaatactatgcagactccgtgaagggccgattcaccatctccagagacaattccaagaacacgctgtatctgcaaatgaacagcctgagagctgaggacacggccgtgtattactgtgcgagagatagggggctacggttctactttgactactggggccaagggaccacggtcaccgtctcctcaggtggtggtggttctggcggcggcggctccggtggtggtggttctcaggccgtgctgactcagccggcttccctctctgcatctcctggagcatcagccagtctcacctgcaccttgcgcaggggcatcaatgttggtgcctacagtatatactggtaccagcagaagccagggagtcctccccagtatctcctgaggtacaaatcagactcagataagcagcagggctctggagtctccagccgcttctctgcatccaaagatgcttcggccaatgcagggattttactcatctctgggctccagtctgaggatgaggctgactattactgtatgatttggcacagcggcgcttctgcggtgttcggcggagggaccaagttgaccgtcctatccggaggtggtggatccgacgtccaactggtgcagtcaggggctgaagtgaaaaaacctggggcctcagtgaaggtgtcctgcaaggcttctggctacacctttactaggtacacgatgcactgggtaaggcaggcacctggacagggtctggaatggattggatacattaatcctagccgtggttatactaattacgcagacagcgtcaagggccgcttcacaatcactacagacaaatccaccagcacagcctacatggaactgagcagcctgcgttctgaggacactgcaacctattactgtgcaagatattatgatgatcattactgccttgactactggggccaaggcaccacggtcaccgtctcctcaggcgaaggtactagtactggttctggtggaagtggaggttcaggtggagcagacgacattgtactgacccagtctccagcaactctgtctctgtctccaggggagcgtgccaccctgagctgcagagccagtcaaagtgtaagttacatgaactggtaccagcagaagccgggcaaggcacccaaaagatggatttatgacacatccaaagtggcttctggagtccctgctcgcttcagtggcagtgggtctgggaccgactactctctcacaatcaacagcttggaggctgaagatgctgccacttattactgccaacagtggagtagtaacccgctcacgttcggtggcgggaccaaggtggagatcaaatag

SEQ ID NO: 55 - DNA, 1506 bp, Artificial sequence
  Bispecific single chain antibody
gaggtgcagctggtcgagtctgggggaggcttggtccagcctgggaggtccctgagactctcctgtgcagcgtctggattcaccgtcagtagctactggatgcactgggtccgccaagctccagggaaggggctggaatgggtaggtttcattagaaacaaagctaatggtgggacaacagaatacgccgcgtctgtgaaaggcagattcaccatctcaagagatgattccaagaacacgctgtatcttcaaatgaacagcctgagagccgaggacacggccgtgtattactgtgcaagagatagggggctacggttctactttgactactggggccaagggaccacggtcaccgtctcctcaggtggtggtggttctggcggcggcggctccggtggtggtggttctcaggccgtgctgactcagccggcttccctctctgcatctcctggagcatcagccagtctcacctgcaccttgcgcaggggcatcaatgttggtgcctacagtatatactggtaccagcagaagccagggagtcctccccagtatctcctgaggtacaaatcagactcagataagcagcagggctctggagtctccagccgcttctctgcatccaaagatgcttcggccaatgcagggattttactcatctctgggctccagtctgaggatgaggctgactattactgtatgatttggcacagcggcgcttctgcggtgttcggcggagggaccaagttgaccgtcctatccggaggtggtggatccgacgtccaactggtgcagtcaggggctgaagtgaaaaaacctggggcctcagtgaaggtgtcctgcaaggcttctggctacacctttactaggtacacgatgcactgggtaaggcaggcacctggacagggtctggaatggattggatacattaatcctagccgtggttatactaattacgcagacagcgtcaagggccgcttcacaatcactacagacaaatccaccagcacagcctacatggaactgagcagcctgcgttctgaggacactgcaacctattactgtgcaagatattatgatgatcattactgccttgactactggggccaaggcaccacggtcaccgtctcctcaggcgaaggtactagtactggttctggtggaagtggaggttcaggtggagcagacgacattgtactgacccagtctccagcaactctgtctctgtctccaggggagcgtgccaccctgagctgcagagccagtcaaagtgtaagttacatgaactggtaccagcagaagccgggcaaggcacccaaaagatggatttatgacacatccaaagtggcttctggagtccctgctcgcttcagtggcagtgggtctgggaccgactactctctcacaatcaacagcttggaggctgaagatgctgccacttattactgccaacagtggagtagtaacccgctcacgttcggtggcgggaccaaggtggagatcaaatag

SEQ ID NO: 56 - DNA, 1500 bp, Artificial sequence
  Bispecific single chain antibody
gaggtgcagctggtcgagtctgggggaggcgtggtccagcctgggaggtccctgagactctcctgtgcagcctctggattcaccctcagtacctatgccatgcactgggtccgccaggctccaggcaaggggctggagtgggtggcacttatatcaaatgatggaagcaataaatactatgcagactccgtgaagggccgattcaccatctccagagacaattccaagaacacgctgtatctgcaaatgaacagcctgagagctgaggacacggctgtgtattactgtactagagatagggggctacggttctactttgactactggggccaagggaccacggtcaccgtctcctcaggtggtggtggttctggcggcggcggctccggtggtggtggttctcaggccgtgctgactcagccggcttccctctctgcatctcctggagcatcagccagtctcacctgcaccttgcgcaggggcatcaatgttggtgcctacagtatatactggtaccagcagaagccagggagtcctccccagtatctcctgaggtacaaatcagactcagataagcagcagggctctggagtctccagccgcttctctgcatccaaagatgcttcggccaatgcagggattttactcatctctgggctccagtctgaggatgaggctgactattactgtatgatttggcacagcggcgcttctgcggtgttcggcggagggaccaagttgaccgtcctatccggaggtggtggatccgacgtccaactggtgcagtcaggggctgaagtgaaaaaacctggggcctcagtgaaggtgtcctgcaaggcttctggctacacctttactaggtacacgatgcactgggtaaggcaggcacctggacagggtctggaatggattggatacattaatcctagccgtggttatactaattacgcagacagcgtcaagggccgcttcacaatcactacagacaaatccaccagcacagcctacatggaactgagcagcctgcgttctgaggacactgcaacctattactgtgcaagatattatgatgatcattactgccttgactactggggccaaggcaccacggtcaccgtctcctcaggcgaaggtactagtactggttctggtggaagtggaggttcaggtggagcagacgacattgtactgacccagtctccagcaactctgtctctgtctccaggggagcgtgccaccctgagctgcagagccagtcaaagtgtaagttacatgaactggtaccagcagaagccgggcaaggcacccaaaagatggatttatgacacatccaaagtggcttctggagtccctgctcgcttcagtggcagtgggtctgggaccgactactctctcacaatcaacagcttggaggctgaagatgctgccacttattactgccaacagtggagtagtaacccgctcacgttcggtggcgggaccaaggtggagatcaaatag

SEQ ID NO: 57 - DNA, 1524 bp, Artificial sequence
  Bispecific single chain antibody
gacgtccaactggtgcagtcaggggctgaagtgaaaaaacctggggcctcagtgaaggtgtcctgcaaggcttctggctacacctttactaggtacacgatgcactgggtaaggcaggcacctggacagggtctggaatggattggatacattaatcctagccgtggttatactaattacgcagacagcgtcaagggccgcttcacaatcactacagacaaatccaccagcacagcctacatggaactgagcagcctgcgttctgaggacactgcaacctattactgtgcaagatattatgatgatcattactgccttgactactggggccaaggcaccacggtcaccgtctcctcaggcgaaggtactagtactggttctggtggaagtggaggttcaggtggagcagacgacattgtactgacccagtctccagcaactctgtctctgtctccaggggagcgtgccaccctgagctgcagagccagtcaaagtgtaagttacatgaactggtaccagcagaagccgggcaaggcacccaaaagatggatttatgacacatccaaagtggcttctggagtccctgctcgcttcagtggcagtgggtctgggaccgactactctctcacaatcaacagcttggaggctgaagatgctgccacttattactgccaacagtggagtagtaacccgctcacgttcggtggcgggaccaaggtggagatcaaatccggaggtggtggatccgaggtgcagctggtcgagtctgggggaggcttggtccagcctgggaggtccctgagactctcctgtgcagcgtctggattcaccgtcagtagctactggatgtactgggtccgccaagctccagggaaggggctggaatgggtaggtttcattctcaacaaagctaatggtggaacaacagaatacgccgcgtctgtgaaaggcagattcaccatctcaagagatgattccaagaacacgctgtatcttcaaatgaacagcctgagagccgaggacacggccgtgtattactgtgcaagagatagggggctacggttctactttgactactggggccaagggaccacggtcaccgtctcctcaggtggtggtggttctggcggcggcggctccggtggtggtggttctgagctcgtgctgactcagccggcttccctctctgcatctcctggagcatcagccagtctcacctgcaccttgcgcaggggcatcaatgttggtgcctacagtatatactggtaccagcagaagccagggagtcctccccagtatctcctgaggtacaaatcagactcagataagcagcagggctctggagtctccagccgcttctctgcatccaaagatgcttcggccaatgcagggattttactcatctctgggctccagtctgaggatgaggctgactattactgtatgatttggcacagcggcgcttctgcggtgttcggcggagggaccaagttgaccgtcctacatcatcaccatcatcattag

SEQ ID NO: 58 - protein, 207 aa, Homo sapiens
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 59 - protein, 171 aa, Homo sapiens
MEHSTFLSGLVLATLLSQVSPFKIPIEELEDRVFVNCNTSITWVEGTVGTLLSDITRLDLGKRILDPRGIYRCNGTDIYKDKESTVQVHYRMCQSCVELDPATVAGIIVTDVIATLLLALGVFCFAGHETGRLSGAADTQALLRNDQVYQPLRDRDDAQYSHLGGNWARNK

SEQ ID NO: 60 - protein, 182 aa, Homo sapiens
MEQGKGLAVLILAIILLQGTLAQSIKGNHLVKVYDYQEDGSVLLTCDAEAKNITWFKDGKMIGFLTEDKKKWNLGSNAKDPRGMYQCKGSQNKSKPLQVYYRMCQNCIELNAATISGFLFAEIVSIFVLAVGVYFIAGQDGVRQSRASDKQTLLPNDQLYQPLKDREDDQYSHLQGNQLRRN

SEQ ID NO: 61 - protein, 164 aa, Homo sapiens
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 62 - protein, 107 aa, Mus musculus
DIVMTQSHKLMSTSVGDRVSITCKASQDVGTAVAWYQQKPGQSPKLLIYWASTRHTGVPDRFTGSGSGTDFTLTISNVQSEDLADYFCQQDSSYPLTFGAGTKVELK

SEQ ID NO: 63 - protein, 118 aa, Mus musculus
EVKLQESGPSLVKPSQTLSLTCSVTGYSITSDYWNWIRKFPGNKLEYVGYISYTGSTYYNPSLKSRISITRDTSKNQYYLQLNSVTSEDTATYYCARYGGWLSPFDYWGQGTTLTVSS

SEQ ID NO: 64 - protein, 107 aa, Mus musculus
DIVTTQSHKLMSTSVGDRVSITCKASQDVGTAVAWYQQKPGQSPKLLIYWASTRHTGVPDRFTGSGSGTDFTLTISNVQSEDLADYFCQQDSSYPLTFGAGTKVELK

SEQ ID NO: 65 - protein, 120 aa, Mus musculus
EVQLQESGPGLVAPSQSLSITCTVSGFSLTTYSINWIRQPPGKGLEWLGVMWAGGGTNSNSVLKSRLIISKDNSKSQVFLKMNSLQTDDTARYYCARYYGNSPYYAIDYWGQGTSVTVSS

SEQ ID NO: 66 - protein, 113 aa, Mus musculus
DIVMTQSPSSLAVSVGEKVSMGCKSSQSLLYSSNQKNSLAWYQQKPGQSPKLLIDWASTRESGVPDRFTGSGSGTDFTLTISSVKAEDLAVYYCQQYYGYPLTFGAGTKLELK

SEQ ID NO: 67 - protein, 118 aa, Mus musculus
EVKLQESGPSLVKPSQTLSLTCSVTGYSIISDYWNWIRKFPGNKLEYLGYISYTGSTYYNPSLKSRISITRDTSKNQYYLQLNSVTTEDTATYYCARRGGWLLPFDYWGQGTTLTVSS

SEQ ID NO: 68 - protein, 106 aa, Mus musculus
DIVMTQSPAIMSASPGEKVTMTCSASSSIRYMHWYQQKPGTSPKRWISDTSKLTSGVPARFSGSGSGTSYALTISSMEAEDAATYYCHQRSSYPWTFGGGTKLEIK

SEQ ID NO: 69 - protein, 118 aa, Mus musculus
EVKLQESGPSLVKPGASVKLSCKASGYTFTSYDINWVKQRPGQGLEWIGWIFPRDNNTKYNENFKGKATLTVDTSSTTAYMELHSLTSEDSAVYFCTKENWVGDFDYWGQGTTLTLSS

SEQ ID NO: 70 - protein, 106 aa, Artificial Sequence
  Humanized Anti-Human B7-H1 Light Chain LC1_1
EIVLTQSPATLSLSPGERATLSCRASSSVSYIYWFQQKPGQAPRLLIYAAFNRATGIPARFSGSGSGTDYTLTISSLEPEDFAVYYCQQWSNNPLTFGQGTKVEIK

SEQ ID NO: 71 - protein, 106 aa, Artificial Sequence
  Humanized Anti-Human B7-H1 Light Chain LC1_2
EIVLTQSPATLSLSPGERATLSCRASSSVSYIYWFQQKPGQSPRPLIYAAFNRATGIPARFSGSGSGTDYTLTISSLEPEDFAVYYCQQWSNNPLTFGQGTKVEIK

SEQ ID NO: 72 - protein, 106 aa, Artificial Sequence
  Humanized Anti-Human B7-H1 Light Chain LC1_3
QIVLTQSPATLSLSPGERATLSCRASSSVSYIYWFQQKPGQSPRPLIYATFNLASGIPARFSGSGSGTSYTLTISRLEPEDFAVYYCQQWSNNPLTFGQGTKVEIK

SEQ ID NO: 73 - protein, 106 aa, Artificial Sequence
  Humanized Anti-Human B7-H1 Light Chain LC2_1
DIQLTQSPSSLSASVGDRVTITCRASSGVSYIYWFQQKPGKAPKLLIYAAFNLASGVPSRFSGSGSGTEYTLTISSLQPEDFATYYCQQWSNNPLTFGQGTKVEIK

SEQ ID NO: 74 - protein, 106 aa, Artificial Sequence
  Humanized Anti-Human B7-H1 Light Chain LC2_2
DIQLTQSPSSLSASVGDRVTITCRASSGVSYIYWFQQKPGKAPKPLIYAAFNLASGVPSRFSGSGSGTEYTLTISSLQPEDFATYYCQQWSNNPLTFGQGTKVEIK

SEQ ID NO: 75 - protein, 106 aa, Artificial Sequence
  Humanized Anti-Human B7-H1 Light Chain LC2_3
DIQLTQSPSILSASVGDRVTITCRASSSVSYIYWFQQKPGKAPKPLIYATFNLASGVPSRFSGSGSGTSYTLTISSLQPEDFATYYCQQWSNNPLTFGQGTKVEIK

SEQ ID NO: 76 - protein, 114 aa, Artificial Sequence
  Humanized Anti-Human B7-H1 Heavy Chain HC1_1
QVQLVQSGAEVKKPGASVKVSCKASGYTFPDYYMNWVRQAPGQGLEWMGDIDPNYGGTNYAQKFQGRVTMTRDTSISTAYMELSRLRSDDTAVYYCARGALTDWGQGTMVTVSS

SEQ ID NO: 77 - protein, 114 aa, Artificial Sequence
  Humanized Anti-Human B7-H1 Heavy Chain HC1_2
QVQLVQSGAEVKKPGASVKVSCKASGYTFPDYYMNWVRQAPGQSLEWMGDIDPNYGGTNYNQKFQGRVTMTRDTSISTAYMELSRLRSDDTAVYYCARGALTDWGQGTMVTVSS

SEQ ID NO: 78 - protein, 114 aa, Artificial Sequence
  Humanized Anti-Human B7-H1 Heavy Chain HC1_3
EVQLVQSGAEVKKPGASVKVSCKASGYTFPDYYMNWVRQAPGQSLEWMGDIDPNYGGTNYNQKFQGRVTMTVDRSSSTAYMELSRLRSDDTAVYYCARGALTDWGQGTMVTVSS

SEQ ID NO: 79 - protein, 114 aa, Artificial Sequence
  Humanized Anti-Human B7-H1 Heavy Chain HC2_1
EVQLVESGGGLVQPGRSLRLSCTASGYTFPDYYMNWVRQAPGKGLEWVGDIDPNYGGTTYAASVKGRFTISVDRSKSIAYLQMSSLKTEDTAVYYCTRGALTDWGQGTMVTVSS

SEQ ID NO: 80 - protein, 114 aa, Artificial Sequence
  Humanized Anti-Human B7-H1 Heavy Chain HC2_2
EVQLVESGGGLVQPGRSLRLSCTASGYTFPDYYMNWVRQAPGKGLEWVGDIDPNYGGTTYNASVKGRFTISVDRSKSIAYLQMSSLKTEDTAVYYCARGALTDWGQGTMVTVSS

SEQ ID NO: 81 - protein, 114 aa, Artificial Sequence
  Humanized Anti-Human B7-H1 Heavy Chain HC2_3
EVQLVESGGGLVQPGRSLRLSCTASGYTFPDYYMNWVRQAPGKGLEWVGDIDPNYGGTTYNQSVKGRFTISVDRSKSIAYLQMSSLKTEDTAVYYCARGALTDWGQGTMVTVSS

SEQ ID NO: 82 - protein, 106 aa, Mus musculus
DIQMTQFPSSLCASQGGKVTVTCKASQDINNYMAWYQHKPGKGPRLLIHYTSTLLSGIPSRFSGSGSGRDYSFSISNLEPEDIATYYCLQYDNLWTFGGGTKLEIK

SEQ ID NO: 83 - protein, 119 aa, Mus musculus
EVQLQQSGPVLVKPGASVKMSCKASGYTFTDYYMNWVKQSHGKSLEWIGNINPYNGGTTYNQKFKGKATLTVDKSSRTAYMEINSLTSEDSAVYYCARGRIYDGSLDYWGQGTALTVSS

SEQ ID NO: 84 - protein, 107 aa, Mus musculus
DIVMTQSQKFMSTSVGDRVSVTCKASQSVDTNVAWYQQKPGQSPKALIFSASYRYSGVPDRFTGSGSGTDFTLTINSVQSEDLAEYFCQQYNSYPYTFGSGTKLEIK

SEQ ID NO: 85 - protein, 116 aa, Mus musculus
QVQLQQSGAELAKPGASVRLSCKASGYTFTNYWMHWVKQRPGQGLEWIGHINPSSGFTTYNQNFKDKATLTADKSSNTAYMQLSSLTYEDSAVYFCAREDYDVDYWGQGTTLTVSS

SEQ ID NO: 86 - protein, 106 aa, Mus musculus
QIVLTQSPALMSASPGEKVTMTCSASSSVSYMYWYQQKPRSSPKPWIYLTSNLASGVPARFSGSGSGTSYSLTISSMEAEDAATYYCQQWSSNPFTFGSGTKLEIK

SEQ ID NO: 87 - protein, 122 aa, Mus musculus
EVQLVESGGGLVKPGGSLKLSCAASGFTFSDYGMHWVRQAPEKGLEWVAYISSGSYTIYYTDTVKGRFTISRDNAKNTLFLQMTSLRSEDTAMYYCARRGYGSFYEYYFDYWGQGTTLTVSS

SEQ ID NO: 88 - protein, 106 aa, Artificial Sequence
  Humanized Anti-Human PD-1 Light Chain LC1_1
EIVLTQSPATLSLSPGERATLSCRASSSVSYMYWYQQKPGQAPRLLIYLASNRATGIPARFSGSGSGTDYTLTISSLEPEDFAVYYCQQWSSNPFTFGQGTKLEIK

SEQ ID NO: 89 - protein, 106 aa, Artificial Sequence
  Humanized Anti-Human PD-1 Light Chain LC1_2
QIVLTQSPATLSLSPGERATLSCSASSSVSYMYWYQQKPGQAPRLLIYLTSNRATGIPARFSGSGSGTDYTLTISSLEPEDFAVYYCQQWSSNPFTFGQGTKLEIK

SEQ ID NO: 90 - protein, 106 aa, Artificial Sequence
  Humanized Anti-Human PD-1 Light Chain LC2_1
DIQLTQSPSSLSASVGDRVTITCRASSSVSYMYWYQQKPGKAPKLLIYLASNLASGVPSRFSGSGSGTEYTLTISSLEPEDFATYYCQQWSSNPFTFGQGTKLEIK

SEQ ID NO: 91 - protein, 106 aa, Artificial Sequence
  Humanized Anti-Human PD-1 Light Chain LC2_2
QIQLTQSPSSLSASVGDRVTITCSASSSVSYMYWYQQKPGKAPKLLIYLTSNLASGVPSRFSGSGSGTEYTLTISSLEPEDFATYYCQQWSSNPFTFGQGTKLEIK

SEQ ID NO: 92 - protein, 122 aa, Artificial Sequence
  Humanized Anti-Human PD-1 Heavy Chain HC1_1
EVQLVESGGGLVQPGGSLRLSCAASGFTFSDYGMHWVRQAPGKGLEWVSYISSGSSTIYYADSVKGRFTISRDNAKNTLYLQMSSLRAEDTAVYYCARRGYGSFYEYYFDYWGQGTTVTVSS

SEQ ID NO: 93 - protein, 122 aa, Artificial Sequence
  Humanized Anti-Human PD-1 Heavy Chain HC1_2
EVQLVESGGGLVQPGGSLRLSCAASGFTFSDYGMHWVRQAPGKGLEWVAYISSGSYTIYYADSVKGRFTISRDNAKNTLYLQMSSLRAEDTAVYYCARRGYGSFYEYYFDYWGQGTTVTVSS

SEQ ID NO: 94 - protein, 122 aa, Artificial Sequence
  Humanized Anti-Human PD-1 Heavy Chain HC1_3
EVQLVESGGGLVQPGGSLRLSCAASGFTFSDYGMHWVRQAPGKGLEWVAYISSGSYTIYSADSVKGRFTISRDNAKNTLYLQMSSLRAEDTAVYYCARRGYGSFYEYYFDYWGQGTTVTVSS

SEQ ID NO: 95 - protein, 122 aa, Artificial Sequence
  Humanized Anti-Human PD-1 Heavy Chain HC2_1
QVQLVQSGAEVKKPGASVKVSCKASGFTFSDYGMHWVRQAPGQRLEWMGYISSGSSTIYYSQKFQGRVTITRDNSASTLYMELSSLRSEDTAVYYCARRGYGSFYEYYFDYWGQGTTLTVSS

SEQ ID NO: 96 - protein, 122 aa, Artificial Sequence
  Humanized Anti-Human PD-1 Heavy Chain HC2_2
EVQLVQSGAEVKKPGASVKVSCAASGFTFSDYGMHWVRQAPGQRLEWMGYISSGSYTIYYSQKFQGRVTITRDNSASTLYMELSSLRSEDTAVYYCARRGYGSFYEYYFDYWGQGTTLTVSS

SEQ ID NO: 97 - protein, 122 aa, Artificial Sequence
  Humanized Anti-Human PD-1 Heavy Chain HC2_3
EVQLVQSGAEVKKPGASVKVSCAASGFTFSDYGMHWVRQAPGQRLEWVAYISSGSYTIYYSQKFQGRVTITRDNSASTLYMELSSLRSEDTAVYYCARRGYGSFYEYYFDYWGQGTTLTVSS

SEQ ID NO: 98 - protein, 152 aa, Homo sapiens
QSPSSLSASVGDRVTITCRASQSINTYLIWYQQKPGKAPNFLISATSILQSGVPSRFRGSGSGTNFTLTINSLHPEDFATYYCQQSYSTPFTFGPGTKVDIKRTVAAPSVFIFPPSDEQLKSGTASVVCLLNNFYPREAKVQWKVDNALQSG

SEQ ID NO: 99 - protein, 169 aa, Homo sapiens
GVVQPGRSLRLSCAASGFTFSSYGMHWVRQAPGKGLEWVAVIWYDGSNKYYADSVKGRFTISRDNSKNTLYLQMNSLRAEDTAVYYCARGARIITPCMDVWGQGTTVTVSSASTKGPSVFPLAPCSRSTSESTAALGCLVKDYFPEPVTVSWNSGALTSGVHTFPAVLQ

SEQ ID NO: 100 - protein, 141 aa, Homo sapiens
QSPGTLSLSPGERATLSCRASQSISSSFLAWYQQRPGQAPRLLIYGASSRATGIPDRFSGSGSGTDFTLTISRLEPEDFAVYYCQQYGTSPWTFGQGTKVEIKRTVAAPSVFIFPPSDEQLKSGTASVVCLLNNFYPREAK

SEQ ID NO: 101 - protein, 167 aa, Homo sapiens
GVVQPGRSLRLSCVASGFTFSSHGMHWVRQAPGKGLEWVAVIWYDGRNKYYADSVKGRFTISRDNSKNTLFLQMNSLRAEDTAVYYCARGGHFGPFDYWGQGTLVTVSSASTKGPSVFPLAPCSRSTSESTAALGCLVKDYFPEPVTVSWNSGALTSGVHTFPAVLQ

SEQ ID NO: 102 - protein, 141 aa, Homo sapiens
QSPGTLSLSPGERATLSCRTSVSSSYLAWYQQKPGQAPRLLIYGASSRATGIPDRFSGSGSGTDFTLTISRLEPEDFAVYYCQQYGISPFTFGGGTKVEIKRTVAAPSVFIFPPSDEQLKSGTASVVCLLNNFYPREAKVQ

SEQ ID NO: 103 - protein, 166 aa, Homo sapiens
GVVQPGRSLRLSCTASGFTFSNYGMHWVRQAPGKGLEWVAVIWYDGSNKHYGDSVKGRFTISSDNSKNTLYLQMNSLRAEDTAVYYCARGERLGSYFDYWGQGTLVTVSSASTKGPSVFPLAPCSRSTSESTAALGCLVKDYFPEPVTVSWNSGALTSGVHTFPAV

SEQ ID NO: 104 - protein, 142 aa, Homo sapiens
SPGTLSLSPGERATLSCRASQSISSNFLAWYQQKPGQAPRLLIYRPSSRATGIPDSFSGSGSGTDFTLTISRLEPEDFALYYCQQYGTSPFTFGPGTKVDIKRTVAAPSVFIFPPSDEQLKSGTASVVCLLNNFYPREAKVQ

SEQ ID NO: 105 - protein, 167 aa, Homo sapiens
GVVQPGRSLRLSCVASGFIFSSHGIHWVRQAPGKGLEWVAVIWYDGRNKDYADSVKGRFTISRDNSKNTLYLQMNSLRAEDTAVYYCARVAPLGPLDYWGQGTLVTVSSASTKGPSVFPLAPCSRSTSESTAALGCLVKDYFPEPVTVSWNSGALTSGVHTFPAVLQ

SEQ ID NO: 106 - protein, 139 aa, Homo sapiens
GTLSLSPGERATLSCRASQSVSSYLAWYQQKPGQAPRLLIYGASSRATGIPDRFSGSGSGTDFTLTISRLEPEDFAVYYCQQYGRSPFTFGPGTKVDIKRTVAAPSVFIFPPSDEQLKSGTASVVCLLNNFYPREAKVQ

SEQ ID NO: 107 - protein, 163 aa, Homo sapiens
PGRSLRLSCAASGFTFSSHGIHWVRQAPGKGLEWVAVIWYDGRNKDYADSVKGRFTISRDNSKKTLYLQMNSLRAEDTAVYYCARVAPLGPLDYWGQGTLVTVSSASTKGPSVFPLAPCSRSTSESTAALGCLVKDYFPEPVTVSWNSGALTSGVHTFPAVLQ

SEQ ID NO: 108 - protein, 142 aa, Homo sapiens
QSPGTLSLSPGERATLSCRASQSVSSYLAWYQQKPGQAPRPLIYGVSSRATGIPDRFSGSGSGTDFTLTISRLEPEDFAVYYCQQYGISPFTFGPGTKVDIKRTVAAPSVFIFPPSDEQLKSGTASVVCLLNNFYPREAKVQ

SEQ ID NO: 109 - protein, 138 aa, Homo sapiens
GGVVEPGRSLRLSCTASGFTFSSYGMHWVRQAPGKGLEWVAVIWYDGSNKHYADSAKGRFTISRDNSKNTLYLQMNSLRAEDTAVYYCARAGLLGYFDYWGQGTLVTVSSASTKGPSVFPLAPCSRSTSESTAALGCL

SEQ ID NO: 110 - protein, 146 aa, Homo sapiens
QSPGTLSLSPGERATLSCRASQSVSSYLAWYQQKPGQAPRLLIYGASSRATGIPDRFSGSGSGTDFTLTISRLEPEDFAVYYCQQYGRSPFTFGPGTKVDIKRTVAAPSVFIFPPSDEQLKSGTASVVCLLNNFYPREAKVQWKGG

SEQ ID NO: 111 - protein, 153 aa, Homo sapiens
PGRSLRLSCAASGFTFSSHGIHWVRQAPGKGLEWVAVIWYDGRNKDYADSVKGRFTISRDNSKNTLYLQMNSLRAEDTAVYYCARVAPLGPLDYWGQGTLVTVSSASTKGPSVFPLAPCSRSTSESTAALGCLVKDYFPEPVTVSWNSGALTS

SEQ ID NO: 112 - protein, 139 aa, Homo sapiens
PSSLSASVGDRVTITCRASQSINSYLDWYQQKPGKAPKLLIYAASSLQSGVPSRFSGSGSGTDFTLTISSLQPEDFATYYCQQYYSTPFTFGPGTKVEIKRTVAAPSVFIFPPSDEQLKSGTASVVCLLNNFYPREAKV

SEQ ID NO: 113 - protein, 167 aa, Homo sapiens
GVVQPGRSLRLSCAASGFTFSSYGMHWVRQAPGKGLEWVAVIWYDGSNKYYADSVKGRFTISRDNSKNTLYLQMNSLRAEDTAVYYCARDPRGATLYYYYYGMDVWGQGTTVTVSSASTKGPSVFPLAPCSRSTSESTAALGCLVKDYFPEPVTVSWNSGALTSGVH

SEQ ID NO: 114 - protein, 134 aa, Homo sapiens
TQSPSSLSASVGDRVTITCRASQNISRYLNWYQQKPGKAPKFLIYVASILQSGVPSGFSASGSGPDFTLTISSLQPEDFATYYCQQSYSTPFTFGPGTKVDIKRTVAAPSVFIFPPSDEQLKSGTASVVCLLNN

SEQ ID NO: 115 - protein, 150 aa, Homo sapiens
GVVQPGRSLRLSCAASGFTFSSYGMHWVRQAPGKGLEWVAVIWYDGSHKYYADSVKGRFTISRDNSKNTLYLQMNSLRAEDTAVYYCARGAVVVPAAMDVWGQGTTVTVSSASTKGPSVFPLAPCSRSTSESTAALGCLVKDYFPEPVTV

SEQ ID NO: 116 - protein, 150 aa, Homo sapiens
TQSPSSLSASVGDRVTITCRASQSICNYLNWYQQKPGKAPRVLIYAASSLQGGVPSRFSGSGSGIDCTLTISSLQPEDFATYYCQQSYITPFTFGPGTRVDIERTVAAPSVFIFPPSDEQLKSGTASVVCLLNNFYPREAKVQWKVDNAY

SEQ ID NO: 117 - protein, 146 aa, Homo sapiens
VVQPGRSLRLSCAASGFTFSSCGMHWVRQAPGKGLEWVAVIWSDGSHKYYADSVKGRFTISRDNSKNTLYLQMNSLRAEDTAVYYCARGTMIVVGTLDYWGQGTLVTVSSASTKGPSVFPLAPCSRSTSESTAALGCLVKDYFPEP

SEQ ID NO: 118 - protein, 139 aa, Homo sapiens
PLSLPVTLGQPASISCRSSQSLVYSDGNTYLNWFQQRPGQSPRRLIYKVSNWDSGVPDRFSGSGSGTDFTLKISRVEAEDVGVYYCMQGSHWPPTFGQGTKVEIKRTVAAPSVFIFPPSDEQLKSGTASVVCLLNNFYP

SEQ ID NO: 119 - protein, 171 aa, Homo sapiens
GVVQPGRSLRLSCAASGFTFSSYGVHWVRQAPGKGLEWVAVIWYDGSNKYYADSVKGRFTISRDNSKSTLYLQMNSLRAEDTAVYYCARDSYYDFWSGRGGMDVWGQGTTVTVSSASTKGPSVFPLAPCSRSTSESTAALGCLVKDYFPEPVTVSWNSGALTSGVHTFPAV

SEQ ID NO: 120 - protein, 133 aa, Homo sapiens
PGEPASISCRSSQSLLHSNGYNYLDWYLQKPGQSPQLLIYLGSNRASGVPDRFSGSGSGTDFTLKLSRVEAEDVGVYYCMQALQTPLTFGGGTKVEIKRTVAAPSVFIFPPSDEQLKSGTASVVCLLNNFYPR

SEQ ID NO: 121 - protein, 163 aa, Homo sapiens
VQPGRSLRLSCAASGFTFSNYAMHWVRQAPGKGLEWVVVIWHDGNNKYYAESVKGRFTISRDNSKNTLYLQMNSLRAEDTAVYYCARDQGTGWYGGFDFWGQGTLVTVSSASTKGPSVFPLAPCSRSTSESTAALGCLVKDYFPEPVTVSWNSGALTSGVHTF